SEQUENCE LISTING (30 sequences)

SEQ ID NO: 1 - DNA, 570 bp, Homo sapiens
accatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtga

SEQ ID NO: 2 - protein, 188 aa, Homo sapiens
  mat_peptide (22)..(188)
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 3 - DNA, 624 bp, Homo sapiens
atgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgccgccgctgccgacgccgcagcttcctccgttgccaagggcggggcttag

SEQ ID NO: 4 - protein, 207 aa, Homo sapiens
  mat_peptide (22)..(207)
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 5 - protein, 13 aa, Artificial sequence
  Synthetic peptide
PXCVXXXRCXGCC

SEQ ID NO: 6 - DNA, 2108 bp, Homo sapiens
  misc_feature (2002): n = a, c, g, or t
ccccgccgtgagtgagctctcaccccagtcagccaaatgagcctcttcgggcttctcctggtgacatctgccctggccggccagagacgagggactcaggcggaatccaacctgagtagtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaagaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcrtgctgataggacagactggatttttcatatttcttattaaaatttctgccatttagaagaagagaactacattcatggtttggaagagataaacctgaaaagaagagtggccttatcttcactttatcgataagtcagtttatttgtttcattgtgtacatttttatattctccttttgacattataactgttggcttttctaatcttgttaaatatatctatttttaccaaaggtatttaatattcttttttatgacaacttagatcaactatttttagcttggtaaatttttctaaacacaattgttatagccagaggaacaaagatggatataaaaatattgttgccctggacaaaaatacatgtatntccatcccggaatggtgctagagttggattaaacctgcattttaaaaaacctgaattgggaanggaanttggtaaggttggccaaancttttttgaaaataattaa

SEQ ID NO: 7 - protein, 345 aa, Homo sapiens [sequence omitted]

SEQ ID NO: 8 - DNA, 2253 bp, Homo sapiens
  CDS (176)..(1288)
cgctcggaaagttcagcatgcaggaagtttggggagagctcggcgattagcacagcgacccgggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgagagcaatcacctcacagacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgcgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaaggtttccagagaaaaggccaatacctaagcattttttccatgagaagcactgcatacttacctatgtggactgtaataacctgtctccaaaaccatgccataataatataagtgctttagaaattaaatcattgtgttttttatgcattttgctgaggcatccttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacgtaactggaaaccg

SEQ ID NO: 9 - protein, 370 aa, Homo sapiens
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 10 - protein, 116 aa, Artificial sequence
  PDGF-C Core Domain
GRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 11 - DNA, 990 bp, Homo sapiens
  CDS (57)..(629)
cagtgtgctggcggcccggcgcgagccggcccggccccggtcgggcctccgaaaccatgaactttctgctgtcttgggtgcattggagcctcgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacaccgcggccgccagcacactg

SEQ ID NO: 12 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 13 - DNA, 1997 bp, Homo sapiens
  CDS (352)..(1608)
cccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 14 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 15 - DNA, 1645 bp, Homo sapiens
  CDS (322)..(768)
gggattcgggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggattctgctggggccgccacggcctggtggtgggaaggccggcagcgggcggaggggattcagccacttccccctcttcttctgaagatcagaacattcagctctggagaacagtggttgcctgggggcttttgccactccttgtcccccgtgatctcccctcacactttgccatttgcttgtactgggacattgttctttccggccgaggtgccaccaccctgcccccactaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaacgc

SEQ ID NO: 16 - protein, 149 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERCGDAVPRR

SEQ ID NO: 17 - DNA, 2029 bp, Homo sapiens
  CDS (411)..(1472)
gttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 18 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 19 - DNA, 1830 bp, Orf virus
  CDS (312)..(755)
cggccacgcggccgcgaactgcgcgctcgcgcgcgtggcgaccgcgctgacgcgccgcgtgcccgcgagccggcacggcctcgcggagggcggcacgccgccgtggacgctgctgctggcggtggccgcggtggcggtgctcggcgtggtggcaatttcgctgctgcgccgcgcgctaagaatacggtttagatactcaaagtctatccagacacttagagtgtaactttgagtaaaaaatgtaaatactaacgccaaaatttcgatagttgttaagcaatatataacatttttaaaacgtcatcaccagcatgaagttaacagctacgttacaagttgttgttgcattgttaatatgtatgtataatttgccagaatgcgtgtctcagagtaatgattcacctccttcaaccaatgactggatgcgtacactagacaaaagtggttgtaaacctagagatactgttgtttatttgggagaagaatatccagaaagcactaacctacaatataatccccggtgcgtaactgttaaacgatgcagtggttgctgtaacggtgacggtcaaatatgtacagcggttgaaacaagaaatacaactgtaacagtttcagtaaccggcgtgtctagttcgtctggtactaatagtggtgtatctactaaccttcaaagaataagtgttacagaacacacaaagtgcgattgtattggtagaacaacgacaacacctacgaccactagggaacctagacgataactaataacaaaaaatgtttatttttgtaaatacttaattattacacactttacaataatctcaaaaataaattgcgtgcccggacggctgcagctggtgacgctgctgtgtcacacactgcgtattcgattcaagttcactaacgccactaaactagttgtgcgtgtccgagtgttaaccgtacgtcaaactaacatcttacctgtccgtgacaagaactaaaacttgaaccacatatttttaaagtatatttaacaaaatcactcacactcacacaatcataaacaccacaaccacaaccaaacacgcatgagaattaatattcttacttatccgtaacactctatgctgtacatcaacgcatcagagcagtctgagtctgactaatggcggcaaacgggaacgcaggcgcgacataatcactgagaatctccgcagcaaccgctcaaggacatctctagcgctaacggctgtttgtcattcccccgtgtgttcatctcacacgacattgtgaccgtcgcaaagcacacattcaaagtgccgcatgtggaagaattcaccgtcgagacacacaccataattaaacaagatcagtgcataagagagattagcattctacagcacaccacgtgcgaatacggacctcgtaattgtttagactagaacacctctggtctaaacaacatgtccgatcttagaacagagtttatgacgcatatgtaactgtgttctttatgtagaagttatcttttatgtcactcccttgtcttagatgagttatacatgacatgatgtatgtgtcgcccgcggcggcgcggggcgctcggcggcggggctgctgcgcgcggcgggcccgcggtggcggcggctggcgcggcgctgcggccgcgggcgcgcggcggggtagcggcccgcccgcccgggcgcccgccgcagcccttgccccggaccaggcgccacggagcaaagtgaaaaaggaccgcctagcagtcgagaccctcccgccgcagccgcgacaccccacacccgccttccacccgccagacgccaacaccacagccaacaagcatgc

SEQ ID NO: 20 - protein, 148 aa, Orf virus
MKLTATLQVVVALLICMYNLPECVSQSNDSPPSTNDWMRTLDKSGCKPRDTVVYLGEEYPESTNLQYNPRCVTVKRCSGCCNGDGQICTAVETRNTTVTVSVTGVSSSSGTNSGVSTNLQRISVTEHTKCDCIGRTTTTPTTTREPRR

SEQ ID NO: 21 - DNA, 851 bp, Orf virus
  CDS (2)..(223)
cggccacgcggccgcgaactgcgcgctcgcgcgcgtggcgaccgcgctgacgcgccgcgtgcccgcgagccggcacggcctcgcggagggcggcacgccgccgtggacgctgctgctggcggtggccgcggtgacggtgctcggcgtggtggcggtttcactgctgcggcgcgcgctgcgggtacgctaccgcttcgcgcggccggccgcgctgcgcgcgtagccgcgcaaaatgtaaattataacgcccaacttttaagggtgaggcgccatgaagttgctcgtcggcatactagtagccgtgtgcttgcaccagtatctgctgaacgcggacagcaacacgaaaggatggtccgaagtgctgaaaggcagcgagtgcaagcctaggccgattgttgttcctgtaagcgagacgcacccagagctgacttctcagcggttcaacccgccgtgtgtcacgttgatgcgatgcggcgggtgctgcaacgacgagagcttggaatgcgtccccacggaagaagtaaacgtgagcatggaactcctgggggcgtcgggctccggtagtaacgggatgcaacgtctgagcttcgtagagcataagaaatgcgattgtagaccacgattcacaaccacgccaccgacgaccacaaggccgcccagaagacgccgctagaactttttatggaccgcagatccaaacgatggatgcgatcaggtacatgcggaagaaggcgccacggagcaaagtgaaaaaggaccgcctagcagtcgagaccctcccgccgcagccgcggacaccccacacccgccttccacccgccagacgccaacaccgcagccaacaagcatgc

SEQ ID NO: 22 - protein, 73 aa, Orf virus
GHAAANCALARVATALTRRVPASRHGLAEGGTPPWTLLLAVAAVTVLGVVAVSLLRRALRVRYRFARPAALRA

SEQ ID NO: 23 - DNA, 2305 bp, Homo sapiens
  CDS (404)..(991)
ttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccggctgcggcgcgcactccctgtttctcctcctcctggctggcgctgcctgcctctccgcactcactgctcgccgggcgccgtccgccagctccgtgctccccgcgccaccctcctccgggccgcgctccctaagggatggtactgaatttcgccgccacaggagaccggctggagcgcccgccccgcgcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccacgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacggatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagagagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcagggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgttttttttttttttttttttttttacaccataaagtgattattaagcttccttttactctttggctagctttttttttttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttcttcttttgttgatccaaaattaaaaaaaaaataccaccaac

SEQ ID NO: 24 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 25 - DNA, 2137 bp, Homo sapiens
  CDS (983)..(1705)
ccctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagcccgcagcccgcagaggacgcccagagcggcgagcgggcgggcagacggaccgacggactcgcgccgcgtccacctgtcggccgggcccagccgagcgcgcagcgggcacgccgcgcgcgcggagcagccgtgcccgccgcccgggcccgccgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctgcactcgggctcgggatccgcccaggtagcggcctcggacccaggtcctgcgcccaggtcctcccctgccccccagcgacggagccggggccgggggcggcggcgccgggggcatgcgggtgagccgcggctgcagaggcctgagcgcctgatcgccgcggacccgagccgagcccacccccctccccagccccccaccctggccgcgggggcggcgcgctcgatctacgcgttcggggccccgcggggccgggcccggagtcggcatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggccttggagtagataatattgtttccctcgtccgtctgtctcgatgcctgattcggacggccaatggtgcctcccccacccctccacgtgtccgtccacccttccatcagcgggtctcctcccagcggcctccggctcttgcccagcagctcaagaagaaaaagaaggactgaactccatcgccatcttcttcccttaactccaagaacttgggataagagtgtgagagagactgatggggtcgctctttgggggaaacgggttccttcccctgcacctggcctgggccacacctgagcgctgtggactgtcctgaggagccctgaggacctctcagcatagcctgcctgatccctgaaccc

SEQ ID NO: 26 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 27 - DNA, 3007 bp, Homo sapiens
  CDS (492)..(1529)
gcccggagagccgcatctattggcagctttgttattgatcagaaactgctcgccgccgacttggcttccagtctggctgcgggcaacccttgagttttcgcctctgtcctgtcccccgaactgacaggtgctcccagcaacttgctggggacttctcgccgctcccccgcgtccccaccccctcattcctccctcgccttcacccccacccccaccacttcgccacagctcaggatttgtttaaaccttgggaaactggttcaggtccaggttttgctttgatccttttcaaaaactggagacacagaagagggctctaggaaaaagttttggatgggattatgtggaaactaccctgcgattctctgctgccagagcaggctcggcgcttccaccccagtgcagccttcccctggcggtggtgaaagagactcgggagtcgctgcttccaaagtgcccgccgtgagtgagctctcaccccagtcagccaaatgagcctcttcgggcttctcctgctgacatctgccctggccggccagagacaggggactcaggcggaatccaacctgagtagtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaagaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcgtgctgataggacagactggatttttcatatttcttattaaaatttctgccatttagaagaagagaactacattcatggtttggaagagataaacctgaaaagaagagtggccttatcttcactttatcgataagtcagtttatttgtttcattgtgtacatttttatattctccttttgacattataactgttggcttttctaatcttgttaaatatatctatttttaccaaaggtatttaatattcttttttatgacaacttagatcaactatttttagcttggtaaatttttctaaacacaattgttatagccagaggaacaaagatgatataaaatattgttgctctgacaaaaatacatgtatttcattctcgtatggtgctagagttagattaatctgcattttaaaaaactgaattggaatagaattggtaagttgcaaagactttttgaaaataattaaattatcatatcttccattcctgttattggagatgaaaataaaaagcaacttatgaaagtagacattcagatccagccattactaacctattccttttttggggaaatctgagcctagctcagaaaaacataaagcaccttgaaaaagacttggcagcttcctgataaagcgtgctgtgctgtgcagtaggaacacatcctatttattgtgatgttgtggttttattatcttaaactctgttccatacacttgtataaatacatggatatttttatgtacagaagtatgtctcttaaccagttcacttattgtactctggcaatttaaaagaaaatcagtaaaatattttgcttgtaaaatgcttaatatcgtgcctaggttatgtggtgactatttgaatcaaaaatgtattgaatcatcaaataaaagaatgtggctattttggggagaaaatt

SEQ ID NO: 28 - protein, 345 aa, Homo sapiens
MSLFGLLLLTSALAGQRQGTQAESNLSSKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 29 - DNA, 399 bp, Orf virus
  CDS (1)..(399)
atgaagtttctcgtcggcatactggtagctgtgtgcttgcaccagtatctgctgaacgcggacagcacgaaaacatggtccgaagtgtttgaaaacagcgggtgcaagccaaggccgatggtctttcgagtacacgacgagcacccggagctaacttctcagcggttcaacccgccgtgtgtcacgttgatgcgatgcggcgggtgctgcaacgacgagagcttagaatgcgtccccacggaagaggcaaacgtaacgatgcaactcatgggagcgtcggtctccggtggtaacgggatgcaacatctgagcttcgtagagcataagaaatgcgattgtaaaccaccactcacgaccacgccaccgacgaccacaaggccgcccagaagacgccgctag

SEQ ID NO: 30 - protein, 132 aa, Orf virus
MKFLVGILVAVCLHQYLLNADSTKTWSEVFENSGCKPRPMVFRVHDEHPELTSQRFNPPCVTLMRCGGCCNDESLECVPTEEANVTMQLMGASVSGGNGMQHLSFVEHKKCDCKPPLTTTPPTTTRPPRRRR

(19) United States
(12) Patent Application Publication
(10) Pub. No.: US 2004/0248796 A1
(43) Pub. Date: Dec. 9, 2004

(54) VEGF-B and PDGF modulation of stem cells

(76) Inventors: Kari Alitalo, Helsinki (FI); Ulf Eriksson, Stockholm (SE); Peter Carmeliet, Leuven (BE); Xuri Li, Stockholm (SE); Desire Collen, Leuven (BE); Seppo Yla-Herttuala, Kuopio (FI); Petri Salven, Helsinki (FI); Iiro Rajantie, Helsinki (FI)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/772,927

(22) Filed: Feb. 4, 2004

Related U.S. Application Data
(60) Provisional application No. 60/445,021, filed on Feb. 4, 2003.
(60) Provisional application No. 60/471,412, filed on May 16, 2003.

(51) Int. Cl. G01N 33/574; A61K 38/18

(52) U.S. Cl. 514/12; 435/7.23

(57) ABSTRACT

The present invention provides materials and methods for VEGF-B and PDGF therapy, especially therapy directed at stem cell recruitment, proliferation, and/or differentiation.

### BACKGROUND

This application claims the benefit of U.S. Provisional Application No. 60/445,021, filed Feb. 4, 2003, and U.S. Provisional Application No. 60/471,412, filed May 16, 2003, which are herein incorporated by reference in their entirety.

The platelet dervived growth factor (PDGF) proteins and their receptors (PDGFRs) are involved in regulation of cell proliferation, survival and migration of several cell types. The vascular endothelial growth factor (VEGF) proteins and their receptors (VEGFRs) play important roles in both vasculogenesis, the development of the embryonic vasculature from early differentiating endothelial cells, and angiogenesis, the process of forming new blood vessels from pre-existing ones [Risau, et al., Dev. Biol. 125:441-450 (1988); Zachary, Intl. J. Biochem. Cell. Bio. 30:1169-1174 (1998); Neufeld, et al., FASEB. J. 13:9-22 (1999); Ferrara, J. Mol. Med. 77:527-543 (1999)]. Both processes depend on the tightly controlled endothelial cell proliferation, migration, differentiation, and survival. Dysfunction of the endothelial cell regulatory system is a key feature of cancer and several diseases associated with abnormal angiogenesis, such as proliferative retinopathies, age-related macular degeneration, rheumatoid arthritis, and psoriasis. Understanding of the specific biological function of the key players involved in regulating endothelial cells will lead to more effective therapeutic applications to treat such diseases [Zachary, Intl. J. Biochem. Cell. Bio. 30:1169-1174 (1998); Neufeld et al., FASEB. J 13:9-22 (1999); Ferrara, J. Mol. Med. 77:527-543 (1999)].

Members of the PDGF/VEGF family are characterized by a number of structural motifs including a conserved PDGF motif defined by the sequence: P-[PS]-C—V-X(3)—R—C-[GSTA]-G-C—C, where the brackets indicate a variable position that can be any one of the amino acids within the brackets. The number contained within the parentheses indicates the number of amino acids that separate the “V” and “R” residues. This conserved motif falls within a large domain of 70-150 amino acids defined in part by eight highly conserved cysteine residues that form inter- and intramolecular disulfide bonds. This domain forms a cysteine knot motif composed of two disulfide bonds which form a covalently linked ring structure between two adjacent β strands, and a third disulfide bond that penetrates the ring [see for example, FIG. 1 in Muller et al., Structure 5:1325-1338 (1997)], similar to that found in other cysteine knot growth factors, e.g., transforming growth factor-β (TGF-β). The amino acid sequence of all known PDGF/VEGF proteins, with the exception of VEGF-E, contains the PDGF domain. The PDGF/VEGF family proteins are predominantly secreted glycoproteins that form either disulfide-linked or non-covalently bound homo- or heterodimers whose subunits are arranged in an anti-parallel manner [Stacker and Achen, Growth Factors 17:1-11 (1999); Muller et al., Structure 5:1325-1338 (1997)].

The platelet-derived growth factor (PDGF) subfamily comprises thus far four family members: PDGF-A, PDGF-B, PDGF-C, and PDGF-D. These ligands bind and activate, with distinct selectivity, dimeric complexes of the receptor tyrosine kinases PDGFR-α and PDGFR-β. [Heldin, C. H. & Westermark, B. Physiol Rev 79, 1283-1316 (1999).] PDGFR-A expression on cardiac vascular endothelial cells has been reported to be involved in the local communication among distinct cells in the heart [Edelberg, et al., J Clinical Inves. 102:837-43 (1998)]. The PDGFs regulate cell proliferation, cell survival and chemotaxis of many cell types in vitro (reviewed in [Heldin et al., Biochimica et Biophysica Acta 1378:F79-113 (1998); Carmeliet P et al. Nature 380, 435-9 (1996); Hellström, M. et al. J Cell Biol 153, 543-53. (2001).]. In vivo, the PDGF proteins exert their effects in a paracrine manner since they often are expressed in epithelial (PDGF-A) or endothelial (PDGF-B) cells in close apposition to the PDGF receptor-expressing mesenchyme [reviewed in Alitalo et al., Int Rev Cytology 172:95-127 (1997)]. Overexpression of the PDGFs has been observed in several pathological conditions, including malignancies, atherosclerosis, and fibroproliferative diseases. In tumor cells and cell lines grown in vitro, co-expression of the PDGFs and PDGF receptors generates autocrine loops, which are important for cellular transformation [Betsholtz et al., Cell 39:447-57 (1984); Keating et al., Science 239:914-6 (1988)]. PDGFR-α has a wide expression pattern [Heldin, C. H. & Westermark, B. Physiol. Rev. 79:1283-1316 (1999)].

The importance of the PDGFs as regulators of cell proliferation and cell survival is well illustrated by recent gene targeting studies in mice. Homozygous null mutations for either PDGF-A or PDGF-B are lethal in mice. Approximately 50% of the homozygous PDGF-A deficient mice have an early lethal phenotype, while the surviving animals have a complex postnatal phenotype with lung emphysema due to improper alveolar septum formation, and a dermal phenotype characterized by thin dermis, misshapen hair follicles, and thin hair. PDGF-A is also required for normal development of oligodendrocytes and subsequent myelination of the central nervous system. The PDGF-B deficient mice develop renal, hematological and cardiovascular abnormalities; where the renal and cardiovascular defects, at least in part, are due to the lack of proper recruitment of mural cells (vascular smooth muscle cells, pericytes or mesangial cells) to blood vessels.

PDGF-A and PDGF-B can homodimerize or heterodimerize to produce three different isoforms: PDGF-AA, PDGF-AB, or PDGF-BB. PDGF-A is only able to bind the PDGF α-receptor (PDGFR-α including PDGR-α/α homodimers). PDGF-B can bind both the PDGFR-α and a second PDGF receptor (PDGFR-β). More specifically, PDGF-B can bind to PDGFR-α/α and PDGFR-β/β homodimers, as well as PDGFR-α/β heterodimers. PDGF-C binds PDGR-α/α homodimers and PDGF-D binds PDGFR-β/β homodimers and both have been reported to bind PDGFR-α/β heterodimers.

PDGF-AA and -BB are the major mitogens and chemoattractants for cells of mesenchymal origin, but have no, or little effect on cells of endothelial lineage, although both PDGFR-α and -β are expressed on endothelial cells (EC). PDGF-BB and PDGF-AB have been shown to be involved in the stabilization/maturation of newly formed vessels [Isner, J. M. Nature 415, 234-9. (2002); Vale, P. R., Isner, J. M. & Rosenfield, K. J Interv Cardiol 14, 511-28 (2001); Heldin, C. H. & Westermark, B. Physiol Rev 79, 1283-1316 (1999); Betsholtz, C., Karlsson, L. & Lindahl, P. Bioessays 23, 494-507. (2001)]. Other data however, showed that PDGF-BB and PDGF-AA inhibited bFGF-induced angiogenesis in vivo via PDGFR-α signaling. PDGF-AA is among the most potent stimuli of mesenchymal cell migration, but it either does not stimulate or it minimally stimulates EC migration. In certain conditions, PDGF-AA even inhibits EC migration [Thommen, J Cell Biochem. 1997 Mar. 1;64(3):403-13; De Marchis, F., et al., Blood 99:2045-53 (2002); Cao, R., et al., FASEB. J 16:1575-83 (2002).] Moreover, PDGFR-α has been shown to antagonize the PDGFR-β-induced SMC migration Yu, J., et al., Biochem. Biophys. Res. Commun. 282:697-700 (2001) and neutralizing antibodies against PDGF-AA enhance smooth muscle cell (SMC) migration (Palumbo, R., et al., Arterioscler. Thromb. Vasc. Biol. 22:405-11 (2002). Thus, the angiogenic/arteriogenic activity of the PDGFs, especially when signaling through PDGFR-α, has been controversial and enigmatic.

PDGF-AA and -BB have been reported to play important roles in the proliferation and differentiation of both cardiovascular and neural stem/progenitor cells. PDGF-BB induced differentiation of Flk1+ embryonic stem cells into vascular mural cells [Carmeliet, P., Nature, 2000, 408:43-45; Yamashita, et al., Nature 408:92-6 (2000)], and potently increased neurosphere derived neuron survival [Caldwell, M. A. et al, Nat Biotechnol, 2001, 19:475-479]; while PDGF-AA stimulated oligodendrocyte precursor proliferation through αᵥβ₃ integrins [Baron, et al., Embo. J 21:1957-66 (2002)].

During development, PDGF-C is expressed in muscle progenitor cells and differentiated smooth muscle cells in most organs, including the heart, lung and kidney [Aase, K., et al., Mech. Dev. 110:187-91 (2002)]. In adulthood, PDGF-C is widely expressed in most organs, with the highest expression level in the heart and kidney [Li, X., et al., Nat. Cell. Biol. 2:302-09 (2000)]. PDGF-CC is secreted as an inactive homodimer of approximately 95 kD. Upon proteolytic removal of the CUB domain, PDGF-CC is capable of binding and activating its receptor, PDGFR-α [Li, X. & Eriksson, U., Cytokine & Growth Factor Reviews 244:1-8 (2003)]. In cells co-expressing both PDGFR-α and -β, PDGF-CC may also activate the PDGFR-α/β heterodimer, but not the PDGFR-β/β homodimer [Cao, R., et al., FASEB. J 16:1575-83. (2002); Gilbertson, D. G., et al., J. Biol. Chem. 10:10 (2001)].

Active PDGF-CC is a potent mitogen for fibroblast and vascular smooth muscle cells [Li, et al., Nat. Cell. Biol. 2:302-09 (2000); Cao, et al., FASEB. J 16:1575-83 (2002); Uutela, et al., Circulation 103:2242-7 (2001)]. Both PDGF-AA and PDGF-CC bind PDGFR-α, but only PDGF-CC potently stimulates angiogenesis in mouse cornea pocket and chick chorioallanoic membrane (CAM) assays [Cao, et al., FASEB. J 16:1575-83 (2002)]. PDGF-CC also promotes wound healing by stimulating tissue vascularization [Gilbertson, et al., J. Biol. Chem. 10:10 (2001)]. However, these studies did not address whether PDGF-CC stimulated vessel growth by affecting endothelial or smooth muscle cells, nor did they examine whether PDGF-CC promoted the maturation of newly formed vessels (including vasculogenesis, angiogenesis, neoangiogenesis and arteriogenesis).

The VEGF subfamily is composed of members that share a VEGF homology domain (VHD) characterized by the sequence: C—X(22-24)—P-[PSR]-C-V-X(3)—R—C-[GSTA]-G-C—C—X(6)—C—X(32-41)-C. The VHD domain, determined through analysis of the VEGF subfamily members, comprises the PDGF motif but is more specific. The VEGF subfamily of growth factors and receptors regulate the development and growth of the vascular endothelial system. VEGF family members include VEGF-A, VEGF-B, VEGF-C, VEGF-D and PlGF [Li, X. and U. Eriksson, “Novel VEGF Family Members: VEGF-B, VEGF-C and VEGF-D,” Int. J. Biochem. Cell. Biol., 33(4):421-6 (2001)).]

VEGF-A (or VEGF) was originally purified from several sources on the basis of its mitogenic activity toward endothelial cells, and also by its ability to induce microvascular permeability, hence it is also called vascular permeability factor (VPF). VEGF-A has subsequently been shown to induce a number of biological processes including the mobilization of intracellular calcium, the induction of plasminogen activator and plasminogen activator inhibitor-1 synthesis, promotion of monocyte migration in vitro, induction of antiapoptotic protein expression in human endothelial cells, induction of fenestrations in endothelial cells, promotion of cell adhesion molecule expression in endothelial cells and induction of nitric oxide mediated vasodilation and hypotension [Ferrara, J. Mol. Med. 77: 527-543 (1999); Neufeld, et al., FASEB. J 13:9-22 (1999); Zachary, Intl. J. Biochem. Cell. Bio. 30:1169-74 (1998)].

VEGF-A is a secreted, disulfide-linked homodimeric glycoprotein composed of 23 kD subunits. Five human VEGF-A isoforms of 121, 145, 165, 189 or 206 amino acids in length (VEGF121-VEGF206), encoded by distinct mRNA splice variants, have been described, all of which are capable of stimulating mitogenesis in endothelial cells. However, each isoform differs in biological activity, receptor specificity, and affinity for cell surface- and extracellular matrix-associated heparan-sulfate proteoglycans, which behave as low affinity receptors for VEGF-A. VEGF121 does not bind to either heparin or heparan-sulfate; VEGF145 and VEGF165 (GenBank Acc. No. M32977) are both capable of binding to heparin; and VEGF189 and VEGF206 show the strongest affinity for heparin and heparan-sulfates. VEGF121, VEGF145, and VEGF165 are secreted in a soluble form, although most of VEGF165 is confined to cell surface and extracellular matrix proteoglycans, whereas VEGF189 and VEGF206 remain associated with extracellular matrix. Both VEGF189 and VEGF206 can be released by treatment with heparin or heparinase, indicating that these isoforms are bound to extracellular matrix via proteoglycans. Cell-bound VEGF189 can also be cleaved by proteases such as plasmin, resulting in release of an active soluble VEGF110. Most tissues that express VEGF are observed to express several VEGF isoforms simultaneously, although VEGF121 and VEGF165 are the predominant forms, whereas VEGF206 is rarely detected [Ferrara, J. Mol. Med. 77:527-543 (1999)]. VEGF145 differs in that it is primarily expressed in cells derived from reproductive organs [Neufeld et al., FASEB. J 13:9-22 (1999)].

The pattern of VEGF-A expression suggests its involvement in the development and maintenance of the normal vascular system, and in angiogenesis associated with tumor growth and other pathological conditions such as rheumatoid arthritis. VEGF-A is expressed in embryonic tissues associated with the developing vascular system, and is secreted by numerous tumor cell lines. Analysis of mice in which VEGF-A was knocked out by targeted gene disruption indicate that VEGF-A is critical for survival, and that the development of the cardiovascular system is highly sensitive to VEGF-A concentration gradients. Mice lacking a single copy of VEGF-A die between day 11 and 12 of gestation. These embryos show impaired growth and several developmental abnormalities including defects in the developing cardiovasculature. VEGF-A is also required post-natally for growth, organ development, regulation of growth plate morphogenesis and endochondral bone formation. The requirement for VEGF-A decreases with age, especially after the fourth postnatal week. In mature animals, VEGF-A is required primarily for active angiogenesis in processes such as wound healing and the development of the corpus luteum. [Neufeld, et al., FASEB. J 13:9-22 (1999); Ferrara, J. Mol. Med. 77:527-543 (1999)]. VEGF-A expression is influenced primarily by hypoxia and a number of hormones and cytokines including epidermal growth factor (EGF), TGF-β, and various interleukins. Regulation occurs transcriptionally and also post-transcriptionally such as by increased mRNA stability [Ferrara, J. Mol. Med. 77:527-543 (1999)].

Lack of a single VEGF (VEGF-A) allele results in embryonic lethality (Canneliet, P., et al., Nature, 380(6573):435-39 (1996); and Ferrara, N., et al., Nature, 380(6573):439-42 (1996)). VEGF-A binds to four receptors, VEGFR-1, VEGFR-2, neuropilin-1 and neuropilin-2 (Poltorak, Z., T. Cohen, and G. Neufeld, Herz., 25(2):126-9 (2000)).

PlGF, another member of the VEGF subfamily, is generally a poor stimulator of angiogenesis and endothelial cell proliferation in comparison to VEGF-A, and the in vivo role of PlGF is not well understood. Three isoforms of PlGF produced by alternative mRNA splicing have been described [Hauser, et al., Growth Factors 9:259-268 (1993); Maglione, et al., Oncogene 8:925-931 (1993)]. PlGF forms both disulfide-liked homodimers and heterodimers with VEGF-A. The PlGF-VEGF-A heterodimers are more effective at inducing endothelial cell proliferation and angiogenesis than PlGF homodimers. PlGF is primarily expressed in the placenta, and is also co-expressed with VEGF-A during early embryogenesis in the trophoblastic giant cells of the parietal yolk sac [Stacker and Achen, Growth Factors 17:1-11 (1999)].

For sometime, research on the control of vessel growth focused on VEGF and VEGFR-2, but recently more attention has been given to VEGFR-1 and its ligands besides VEGF, including PlGF and VEGF-B. [Eriksson and Alitalo, Nat. Med. 8:775-777 (2002).] PlGF knock out mice do not experience significant abnormalities in embryonic angiogenesis. However, PlGF deficiency in mice has been reported to impair angiogenesis, plasma extravasation and collateral growth during ischemia, inflammation, wound healing and cancer. [Cammeliet, et al., Nat. Med. 7:575-83 (2001).] Hattori, et al. have reported that PlGF promotes the recruitment of VEGFR-1+hematopoietic stem cells from a quiescent to a proliferative bone marrow microenvironment, contributing to hematopoiesis. [Nat. Med. 8:841-49 (2002).] Luttun and co-workers have reported that PlGF stimulated angiogenesis and collateral growth in ischemic heart and limb with an efficiency comparable to, if not higher than, that of VEGF. [Nat. Med. 8:831-40 (2002).]

The isolation and characteristics of VEGF-B, including nucleotide and amino acid sequences for both human and murine VEGF-B, are described in detail in PCT/US96/02957, and U.S. Pat. Nos. 5,840,693 and 5,607,918 by Ludwig Institute for Cancer Research and Helsinki University Licensing Ltd. Oy and in Olofsson, et al., Proc. Natl. Acad. Sci. USA, 93:2576-2581 (1996). A nucleotide sequence encoding human VEGF-B is also found at GenBank Accession No. U48801. The entire disclosures of the International Patent Application PCT/US97/14696 (WO 98/07832), U.S. Pat. Nos. 5,840,693 and 5,607,918 are incorporated herein by reference.

VEGF-B is very strongly expressed in the heart, and only weakly in the lungs, whereas the reverse is the case for VEGF-A. RT-PCR assays have demonstrated the presence of VEGF-B mRNA in melanoma, normal skin, and muscle. This suggests that VEGF-A and VEGF-B, despite the fact that they are co-expressed in many tissues, have functional differences. A comparison of the PDGF/VEGF family of growth factors reveals that the 167 amino acid isoform of VEGF-B is the only family member that is completely devoid of any glycosylation. Gene targeting studies have shown that VEGF-B deficiency results in mild cardiac phenotype, and impaired coronary vasculature (Bellomo, et al., Circ. Res., 86:E29-35 (2000)).

The human and murine genes for VEGF-B are almost identical, and both span about 4 kb of DNA. The genes are composed of seven exons, and their exon-intron organization resembles that of the VEGF-A and PlGF genes. [Grimmond, et al., Genome Res., 6:124-131 (1996); Olofsson, et al., J. Biol. Chem., 271:19310-17 1996); Townson, et al., Biochem. Biophys. Res. Commun. 220:922-928 (1996).]VEGF-B binds specifically to VEGFR-1 and neuropilin-1. [Olofsson, B., et al., Proc. Nat'l. Acad. Sci. USA, 93(6):2576-81 (1996); Olofsson, B., et al., Proc. Nat'l. Acad. Sci. USA, 95(20):11709-14 (1998).]

VEGF-B displays a unique expression pattern compared with other VEGF family members, with the highest expression level in the cardiac myocytes [Aase, K., et al., Developmental Dynamics, 215(1):12-25 (1999)], whereas VEGFR-1 is expressed in the adjacent endothelial cells [Aase, K., et al., Developmental Dynamics, 215(1):12-25 (1999)], and neuropilin-1 (NP-1) is expressed in both endothelium and cardiac myocytes during development. [Makinen, T., et al., Journal of Biological Chemistry, 274(30):21217-22 (1999); and Kitsukawa, T., et al., Development, 121(12):4309-18 (1995).] The temporal-spatial expression patterns of VEGF-B and its receptors suggest both autocrine and paracrine roles of VEGF-B in the heart.

Both VEGF-B and PlGF exist in two alternatively spliced forms, which differ in their affinity for heparin, and both growth factors are able to form heterodimers with VEGF. Olofsson, et al., Cell Biol., 95:11709-11714 (1998). Although VEGF-B and PlGF both appear to bind exclusively to VEGFR-1 and not VEGFR-2 or VEGFR-3, the two growth factors appear to have different functions. For example, Hattori et al., have reported that PlGF affects hematopoiesis recovery by both binding to VEGFR-1 and by inducing expression of matrix metalloproteinase-9. [Nat. Med. 8:841-49 (2002).] Carmeliet, et al., reported that VEGF-B did not rescue development in PlGF deficient mice. [Nat. Med., 7:575-83 (2001).] The expression of VEGF-B and PlGF are also substantially different with VEGF-B, unlike PlGF, widely expressed and most prominently in heart and skeletal muscle. Furthermore, VEGF residues implicated in VEGFR-1 binding are more highly conserved in VEGF-B than in PlGF. [Olofsson, et al., Cell Biol. 95:11709-11714 (1998).] Proteolytic processing is required for VEGF-B₁₈₆, a VEGF-β isoform discussed below, to bind NP-1, but no such processing is required for PlGF to bind NP-1.

Two VEGF-β isoforms generated by alternative mRNA splicing exist, VEGF-B₁₈₆ and VEGF-B₁₆₇, with the first isoform accounting for about 80% of the total VEGF-B transcripts [Li, X., et al., Growth Factor, 19:49-59 (2001).] [Grimmond, et al., Genome Res., 6:124-131 (1996); Olofsson, et al., J. Biol. Chem., 271:19310-19317 (1996).] The isoforms have an identical N-terminal domain of 115 amino acid residues, excluding the signal sequence. The common N-terminal domain is encoded by exons 1-5. Differential use of the remaining exons 6A, 6B and 7 gives rise to the two splice isoforms. By the use of an alternative splice-acceptor site in exon 6, an insertion of 101 bp introduces a frame-shift and a stop of the coding region of VEGF-B₁₆₇ cDNA. Thus, the two VEGF-β isoforms have differing C-terminal domains.

The two VEGF-β isoforms differ at their carboxy-termini and display different abilities to bind neuropilin-1. [Makinen, et al., J. Biol. Chem., 274(30):21217-22 (1999).] Moreover, VEGF-B₁₈₆ is freely secreted, while VEGF-B₁₆₇ is secreted but largely cell-associated, implying that the functional properties of the two isoforms may be distinct. Both isoforms bind to extracellular matrix tenascin-X and stimulate endothelial cell proliferation through VEGF-receptor-1 (VEGFR-1). [Ikuta, et al., Genes Cells, 5(11):913-927 (2000).]

The different C-terminal domains of the two splice isoforms of VEGF-B affect their biochemical and cell biological properties. The C-terminal domain of VEGF-B₁₆₇ is structurally related to the corresponding region in VEGF, with several conserved cysteine residues and stretches of basic amino acid residues. Thus, this domain is highly hydrophilic and basic and, accordingly, VEGF-B₁₆₇ will remain cell-associated on secretion, unless the producing cells are treated with heparin or high salt concentrations. The cell-associated molecules binding VEGF-B₁₆₇ are likely to be cell surface or pericellular heparin sulfate proteoglycans. It is likely that the cell-association of this isoform occurs via its unique basic C-terminal region. The hydrophobic C-terminal domain of VEGF-B₁₈₆ has no significant similarity with known amino acid sequences in the databases. VEGF-B₁₈₆ is freely secreted from cells [(Olfsson et al., J. Biol. Chem.,271:19310-19317 (1996)] and evidence indicates that this isoform is proteolytically processed, regulating the biological properties of the protein. [Olofsson, et al., Proc. Natl. Acad. Sci. USA, 95:11709-11714 (1998).]

A further difference between the VEGF-β isoforms is found in the glycosylation of the VEGF-β isoforms. VEGF-B₁₆₇ is not glycosylated at all, whereas VEGF-B₁₈₆ is O-glycosylated but not N-glycosylated.

Both isoforms of VEGF-B can form heterodimers with VEGF, consistent with the conservation of the eight cysteine residues involved in inter- and intramolecular disulfide bonding of PDGF-like proteins. Furthermore, co-expression of VEGF-B and VEGF in many tissues suggests that VEGF-B-VEGF heterodimers occur naturally. Heterodimers of VEGF-B₁₆₇-VEGF remain cell-associated. In contrast, heterodimers of VEGF-B₁₈₆ and VEGF are freely secreted from cells in a culture medium. VEGF also forms heterodimers with PlGF. [DiSalvo, et al, J. Biol. Chem. 270:7717-7723 (1995).] The production of heterodimeric complexes between the members of this family of growth factors could provide a basis for a diverse array of angiogenic or regulatory molecules. Enholm, et al., WO 02/36131 report adenovirus gene therapy using a first vector encoding VEGF-B together with a second vector encoding another vascular endothelial growth factor to stimulate angiogenic activity.

A fourth member of the VEGF subfamily, VEGF-C, comprises a VHD that is approximately 30% identical at the amino acid level to VEGF-A. VEGF-C is originally expressed as a larger precursor protein, prepro-VEGF-C, having extensive amino- and carboxy-terminal peptide sequences flanking the VHD, with the C-terminal peptide containing tandemly repeated cysteine residues in a motif typical of Balbiani ring 3 protein. Prepro-VEGF-C undergoes extensive proteolytic maturation involving the successive cleavage of a signal peptide, the C-terminal pro-peptide, and the N-terminal pro-peptide. Secreted VEGF-C protein consists of a non-covalently-linked homodimer, in which each monomer contains the VHD. The intermediate forms of VEGF-C produced by partial proteolytic processing show increasing affinity for the VEGFR-3 receptor, and the mature protein is also able to bind to the VEGFR-2 receptor. [Joukov, et al., EMBO J, 16(13):3898-3911 (1997).] It has also been demonstrated that a mutant VEGF-C, in which a single cysteine at position 156 is either substituted by another amino acid or deleted, loses the ability to bind VEGFR-2 but remains capable of binding and activating VEGFR-3 [International Patent Publication No. WO 98/33917]. In mouse embryos, VEGF-C mRNA is expressed primarily in the allantois, jugular area, and the metanephros. [Joukov, et al., J. Cell. Physiol. 173:211-15 (1997)].

VEGF-C is involved in the regulation of lymphatic angiogenesis: when VEGF-C was overexpressed in the skin of transgenic mice, a hyperplastic lymphatic vessel network was observed, suggesting that VEGF-C induces lymphatic growth [Jeltsch et al., Science, 276:1423-1425 (1997)]. Continued expression of VEGF-C in the adult also indicates a role in maintenance of differentiated lymphatic endothelium [Ferrara, J. Mol. Med. 77:527-543 (1999)]. In addition, VEGF-C shows angiogenic properties: it can stimulate migration of bovine capillary endothelial (BCE) cells in collagen and promote growth of human endothelial cells. [See, e.g., International Patent Publication No. WO 98/33917, incorporated herein by reference.]

VEGF-D is structurally and functionally most closely related to VEGF-C. [See International Patent Publ. No. WO 98/07832, incorporated herein by reference]. Like VEGF-C, VEGF-D is initially expressed as a prepro-peptide that undergoes N-terminal and C-terminal proteolytic processing, and forms non-covalently linked dimers. VEGF-D stimulates mitogenic responses in endothelial cells in vitro. During embryogenesis, VEGF-D is expressed in a complex temporal and spatial pattern, and its expression persists in the heart, lung, and skeletal muscles in adults. Isolation of a biologically active fragment of VEGF-D designated VEGF-DΔNΔC, is described in International Patent Publication No. WO 98/07832, incorporated herein by reference. VEGF-DANAC consists of amino acid residues 93 to 201 of VEGF-D linked to the affinity tag peptide FLAG®.

Four additional members of the VEGF subfamily have been identified in poxviruses, which infect humans, sheep and goats. The orf virus-encoded VEGF-E and NZ2 VEGF are potent mitogens and permeability enhancing factors. Both show approximately 25% amino acid identity to mammalian VEGF-A, and are expressed as disulfide-liked homodimers. Infection by these viruses is characterized by pustular dermatitis which may involve endothelial cell proliferation and vascular permeability induced by these viral VEGF proteins. [Ferrara, J. Mol. Med. 77:527-543 (1999); Stacker and Achen, Growth Factors 17:1-11 (1999)]. VEGF-like proteins have also been identified from two additional strains of the orf virus, D1701 [GenBank Acc. No. AF106020; described in Meyer, et al., EMBO. J 18:363-374 (1999)] and NZ10 [described in International Patent Application PCT/US99/25869, incorporated herein by reference]. These viral VEGF-like proteins have been shown to bind VEGFR-2 present on host endothelium, and this binding is important for development of infection and viral induction of angiogenesis. [Meyer, et al., EMBO. J 18:363-74 (1999); International Patent Application PCT/US99/25869.]

Seven cell surface receptors that interact with PDGF/VEGF family members have been identified. These include PDGFR-α [see e.g., GenBank Acc. No. NM006206], PDGFR-β [see e.g., GenBank Acc. No. NM002609], VEGFR-1/Flt-1 (fms-like tyrosine kinase-1) [GenBank Acc. No. X51602; De Vries, et al., Science 255:989-991 (1992)]; VEGFR-2/KDR/Flk-1 (kinase insert domain containing receptor/fetal liver kinase-1) [GenBank Acc. Nos. X59397 (Flk-1) and L04947 (KDR); Terman, et al., Biochem. Biophys. Res. Comm. 187:1579-1586 (1992); Matthews, et al., Proc. Natl. Acad. Sci. USA 88:9026-9030 (1991)]; VEGFR-3/Flt4 (fms-like tyrosine kinase 4) [U.S. Pat. No. 5,776,755 and GenBank Acc. No. X68203 and S66407; Pajusola et al., Oncogene 9:3545-3555 (1994)]; neuropilin-1 [Gen Bank Acc. No. NM003873], and neuropilin-2 [Gen Bank Acc. No. NM003872]. The two PDGF receptors mediate signaling of PDGFs as described herein. VEGF121, VEGF165, VEGF-B, PlGF-1 and PlGF-2 bind VEGF-R1; VEGF121, VEGF145, VEGF165, VEGF-C, VEGF-D, VEGF-E, and NZ2 VEGF bind VEGF-R2; VEGF-C and VEGF-D bind VEGFR-3; VEGF165, PlGF-2, and NZ2 VEGF bind neuropilin-1; and VEGF165 binds neuropilin-2.[Neufeld, et al., FASEB. J 13:9-22 (1999); Stacker and Achen, Growth Factors 17:1-11 (1999); Ortega, et al., Fron. Biosci. 4:141-152 (1999); Zachary, Intl. J. Biochem. Cell. Bio. 30:1169-1174 (1998); Petrova, et al., Exp. Cell. Res. 253:117-130 (1999)].

The PDGF receptors (including PDGFR-α/α, PDGFR-α/β, and PDGFR-β/β) are protein tyrosine kinase receptors (PTKS) that contain five immunoglobulin-like loops in each of their extracellular domains. VEGFR-1, VEGFR-2, and VEGFR-3 comprise PTKs that are distinguished by the presence of seven Ig domains in their extracellular domain and a split kinase domain in the cytoplasmic region. Both neuropilin-1 and neuropilin-2 are non-PTK VEGF receptors. NP-1 has an extracellular portion includes a MAM domain; regions of homology to coagulation factors V and VIII, MFGPs and the DDR tyrosine kinase; and two CUB-like domains.

Several of the VEGF receptors are expressed as more than one isoform. A soluble isoform of VEGFR-1 lacking the seventh Ig-like loop, transmembrane domain, and the cytoplasmic region is expressed in human umbilical vein endothelial cells. This VEGFR-1 isoform binds VEGF-A with high affinity and is capable of preventing VEGF-A-induced mitogenic responses [Ferrara, J. Mol. Med. 77:527-543 (1999); Zachary, Intl. J. Biochem. Cell. Bio. 30:1169-1174 (1998)]. A C-terminal truncated from of VEGFR-2 has also been reported [Zachary, Intl. J. Biochem. Cell. Bio. 30:1169-1174 (1998)]. In humans, there are two isoforms of the VEGFR-3 protein which differ in the length of their C-terminal ends. Studies suggest that the longer isoform is responsible for most of the biological properties of VEGFR-3.

The receptors for the PDGFs, PDGF α-receptor (PDGFR-α) and the α-receptor (PDGFR-β), are expressed by many in vitro grown cell lines, and they are mainly expressed by mesenchymal cells in vivo.

Gene targeting studies in mice have revealed distinct physiological roles for the PDGF receptors despite the overlapping ligand specificities of the PDGFRs [Rosenkranz, et al., Growth Factors 16:201-16 (1999)]. Homozygous null mutations for either of the two PDGF receptors are lethal. PDGFR-β deficient mice die during embryogenesis at day 10, and show incomplete cephalic closure, impaired neural crest development, cardiovascular defects, skeletal defects, and edemas. The PDGFR-β deficient mice develop similar phenotypes to animals deficient in PDGF-B, that are characterized by renal, hematological and cardiovascular abnormalities; where the renal and cardiovascular defects, at least in part, are due to the lack of proper recruitment of mural cells (vascular smooth muscle cells, pericytes or mesangial cells) to blood vessels.

The expression of VEGFR-1 occurs mainly in vascular endothelial cells, although some may be present on monocytes, trophoblast cells, and renal mesangial cells [Neufeld et al., FASEB. J 13:9-22 (1999)]. High levels of VEGFR-1 mRNA are also detected in adult organs, suggesting that VEGFR-1 has a function in quiescent endothelium of mature vessels not related to cell growth. VEGFR-1−/− mice die in utero between day 8.5 and 9.5. Although endothelial cells developed in these animals, the formation of functional blood vessels was severely impaired, suggesting that VEGFR-1 may be involved in cell-cell or cell-matrix interactions associated with cell migration. Recently, it has been demonstrated that mice expressing a mutated VEGFR-1 in which only the tyrosine kinase domain was missing show normal angiogenesis and survival, suggesting that the signaling capability of VEGFR-1 is not essential. [Neufeld, et al., FASEB. J 13:9-22 (1999); Ferrara, J. Mol. Med. 77:527-543 (1999)].

VEGFR-2 expression is similar to that of VEGFR-1 in that it is broadly expressed in the vascular endothelium, but it is also present in hematopoietic stem cells, megakaryocytes, and retinal progenitor cells [Neufeld, et al., FASEB. J 13:9-22 (1999)]. Although the expression pattern of VEGFR-1 and VEGFR-2 overlap extensively, evidence suggests that, in most cell types, VEGFR-2 is the major receptor through which most of the VEGFs exert their biological activities. Examination of mouse embryos deficient in VEGFR-2 further indicate that this receptor is required for both endothelial cell differentiation and the development of hematopoietic cells [Joukov, et al., J. Cell. Physiol. 173:211-215 (1997)].

VEGFR-3 is expressed broadly in endothelial cells during early embryogenesis. During later stages of development, the expression of VEGFR-3 becomes restricted to developing lymphatic vessels [Kaipainen, A., et al., Proc. Natl. Acad. Sci. USA 92:3566-70 (1995)]. In adults, the lymphatic endothelia and some high endothelial venules express VEGFR-3, and increased expression occurs in lymphatic sinuses in metastatic lymph nodes and in lymphangioma. VEGFR-3 is also expressed in a subset of CD34+hematopoietic cells which may mediate the myelopoietic activity of VEGF-C demonstrated by overexpression studies [WO 98/33917]. Targeted disruption of the VEGFR-3 gene in mouse embryos leads to failure of the remodeling of the primary vascular network, and death after embryonic day 9.5 [Dumont, et al., Science 282:946-49 (1998)]. These studies suggest an essential role for VEGFR-3 in the development of the embryonic vasculature, and also during lymphangiogenesis.

Structural analyses of the VEGF receptors indicate that the VEGF-A binding site on VEGFR-1 and VEGFR-2 is located in the second and third Ig-like loops. Similarly, the VEGF-C and VEGF-D binding sites on VEGFR-2 and VEGFR-3 are also contained within the second Ig-loop [Taipale, et al., Curr. Top. Microbiol. Immunol. 237:85-96 (1999)]. The second Ig-like loop also confers ligand specificity as shown by domain swapping experiments [Ferrara, J. Mol. Med. 77:527-543 (1999)]. Receptor-ligand studies indicate that dimers formed by the VEGF family proteins are capable of binding two VEGF receptor molecules, thereby dimerizing VEGF receptors. The fourth Ig-like loop on VEGFR-1, and also possibly on VEGFR-2, acts as the receptor dimerization domain that links two receptor molecules upon binding of the receptors to a ligand dimer [Ferrara, J. Mol. Med. 77:527-543 (1999)]. Although the regions of VEGF-A that bind VEGFR-1 and VEGFR-2 overlap to a large extent, studies have revealed two separate domains within VEGF-A that interact with either VEGFR-1 or VEGFR-2, as well as specific amino acid residues within these domains that are critical for ligand-receptor interactions. Mutations within either VEGF receptor-specific domain that specifically prevent binding to one particular VEGF receptor have also been recovered [Neufeld, et al., FASEB. J 13:9-22 (1999)].

VEGFR-1 and VEGFR-2 are structurally similar, share common ligands (VEGF121 and VEGF165), and exhibit similar expression patterns during development. However, the signals mediated through VEGFR-1 and VEGFR-2 by the same ligand appear to be slightly different. VEGFR-2 has been shown to undergo autophosphorylation in response to VEGF-A, but phosphorylation of VEGFR-1 under identical conditions was barely detectable. VEGFR-2 mediated signals cause striking changes in the morphology, actin reorganization, and membrane ruffling of porcine aortic endothelial cells recombinantly overexpressing this receptor. In these cells, VEGFR-2 also mediated ligand-induced chemotaxis and mitogenicity; whereas VEGFR-1-transfected cells lacked mitogenic responses to VEGF-A. Mutations in VEGF-A that disrupt binding to VEGFR-2 fail to induce proliferation of endothelial cells, whereas VEGF-A mutants that are deficient in binding VEGFR-1 are still capable of promoting endothelial proliferation. Similarly, VEGF stimulation of cells expressing only VEGFR-2 leads to a mitogenic response whereas comparable stimulation of cells expressing only VEGFR-1 also results in cell migration, but does not induce cell proliferation. In addition, phosphoproteins co-precipitating with VEGFR-1 and VEGFR-2 are distinct, suggesting that different signaling molecules interact with receptor-specific intracellular sequences.

The primary function of VEGFR-1 in angiogenesis may be to negatively regulate the activity of VEGF-A by binding it and thus preventing its interaction with VEGFR-2, whereas VEGFR-2 is thought to be the main transducer of VEGF-A signals in endothelial cells. In support of this hypothesis, mice deficient in VEGFR-1 die as embryos while mice expressing a VEGFR-1 receptor capable of binding VEGF-A but lacking the tyrosine kinase domain survive and do not exhibit abnormal embryonic development or angiogenesis. In addition, analyses of VEGF-A mutants that bind only VEGFR-2 show that they retain the ability to induce mitogenic responses in endothelial cells. However, VEGF-mediated migration of monocytes is dependent on VEGFR-1, indicating that signaling through this receptor is important for at least one biological function. In addition, the ability of VEGF-A to prevent the maturation of dendritic cells is also associated with VEGFR-1 signaling, suggesting that VEGFR-1 may function in cell types other than endothelial cells. [Ferrara, J. Mol. Med. 77:527-543 (1999); Zachary, Intl. J. Biochem. Cell. Bio. 30:1169-1174 (1998)].

Neuropilin-1 was originally cloned as a receptor for the collapsin/semaphorin family of proteins involved in axon guidance [Stacker and Achen, Growth Factors 17:1-11 (1999)]. It is expressed in both endothelia and specific subsets of neurons during embryogenesis, and it thought to be involved in coordinating the developing neuronal and vascular system. Although activation of neuropilin-1 does not appear to elicit biological responses in the absence of the VEGF family tyrosine-kinase receptors, their presence on cells leads to more efficient binding of VEGF165 and VEGFR-2 mediated responses. [Neufeld, et al., FASEB. J. 13:9-22 (1999)] Mice lacking neuropilin-1 show abnormalities in the developing embryonic cardiovascular system. [Neufeld, et al., FASEB. J. 13:9-22 (1999)] Neuropilin-2 was identified by expression cloning and is a collapsin/semaphorin receptor closely related to neuropilin-1. Neuropilin-2 is an isoform-specific VEGF receptor in that it only binds VEGF165. Like neuropilin-1, neuropilin-2 is expressed in both endothelia and specific neurons, and is not predicted to function independently due to its relatively short intracellular domain. The function of neuropilin-2 in vascular development is unknown [Neufeld, et al., FASEB. J. 13:9-22 (1999); WO 99/30157].

Stem cells, also referred to as progenitor cells, comprise both embryonic and adult stem cells. Adult stems cells include, but are not limited to, neural stem cells, hematopoietic stem cells, endothelial stem cells, and epithelial stem cells. See Tepper, et al., Plastic and Reconstructive Surgery, 111:846-854 (2003). Endothelial progenitor cells circulate in the blood and migrate to regions characterized by injured endothelia. Kaushal, et al., Nat. Med., 7:1035-1040 (2001). A small subpopulation of human CD34(+)CD133(+) stem cells from different hematopoietic sources coexpress VEGFR-3 (Salven, et al., Blood, 101(1):168-72 (2003). These cells also have the capacity to differentiate to lymphatic and/or vascular endothelial cells in vitro.

Myelosuppression or bone marrow suppression is a problem experienced by those subjects undergoing chemotherapy and bone marrow transplants. New methods of treating myelosuppression are needed in the art.

There remains a need in the art for new therapies employing VEGF-B and PDGFs or antagonists of VEGF-B and PDGFs. There is also a need in the art to identify growth factors capable of causing mobilization of vascular stem/progenitor cells, increase of stem cell adherence/viability, and promotion of stem cell differentiation for use in therapies.

For therapeutic revascularization of ischemic tissues to succeed, the newly formed vessels must be mature, durable and functional. These requirements imply not only that new endothelium-lined vessels must sprout (“angiogenesis”), but also that these nascent vessels become covered by perivascular smooth muscle cells and/or fibroblasts (“arteriogenesis”), processes that require an involvement of both vascular progenitors and differentiated cells of multiple cardiovascular cell types. Neoangiogenesis and vasculogenesis are also relevant. Vasculogenesis, including adult vasculogenesis, is a process by vascular progenitors are differentiated and mobilized to sites of active vessel growth. While angio/arteriogenesis are easily disregulated by inactivation of candidate genes [Carmeliet, P., et al. Nature 380:435-39 (1996); Hellström, M., et al., J. Cell. Biol. 153:543-53 (2001)], stimulating these processes in a functionally relevant manner has proven to be a much greater challenge than anticipated. A need exists for materials and methods for meeting this goal, preferentially those having pleiotropic activities on both vascular progenitors and differentiated vascular cells of both endothelial and smooth muscle cell lineages.

Neither VEGF, PDGF-AA, PDGF-BB, TGF-β, bFGF nor PlGF has been documented to induce the expression of SMC genes in adult bone marrow-derived progenitors, and very few molecules have been discovered to regulate the differentiation and function of SMC progenitors derived from adult bone marrow (BM) stem cells. [Hirschi, K. K. & Goodell, M. A., Gene Ther. 9:648-52 (2002).] PDGF-BB stimulates embryonic vascular progenitors to acquire a SMC-phenotype [Hirschi, K. K. & Goodell, M. A., Gene Ther. 9:648-52 (2002); Carmeliet, P., Nature 408:43, 45 (2000); Yamashita, J., et al. Nature 408:92-6 (2000)], but is unknown to have similar effects on adult bone marrow-derived progenitors. Thus, there is a need to identify and characterize molecules capable of affecting adult bone marrow-derived progenitors, for use in diagnosis, medicament preparation, and therapy.

### SUMMARY OF THE INVENTION

The present invention relates to new methods of modulating progenitor cell recruitment, proliferation, and/or differentiation, and is based in part on the discovery that VEGF-B and PDGF-C stimulate the recruitment, proliferation and/or differentiation of stem cells. Each of these growth factors may be used alone or in combination with other growth factors as described herein.

In one aspect of the invention VEGF-B is used to stimulate the recruitment, proliferation and/or differentiation of stem cells, including hematopoietic and endothelial precursor cells. In one embodiment, a method of stimulating stem cell recruitment, proliferation, or differentiation is provided which comprises identifying a human subject in need of stem cell recruitment, proliferation, or differentiation, and administering to the human subject a composition comprising a vascular endothelial growth factor B (VEGF-B) product. The term “stem cell recruitment” refers to mobilization of stem cells (e.g., from bone marrow into circulation). The term “proliferation” refers to mitotic reproduction. The term “differentiation” refers to the process by which the pluripotent or multipotent stem cells develop into other cell types. Differentiation may involve a number of stages between pluripotency and fully differentiated cell types, and stimulation through even one stage is considered stimulating differentiation. The terms “proliferation” and “differentiation” are relevant in both in vivo and ex vivo therapies. The recruitment, proliferation, and differentiation are all relevant to the process of myelopoiesis—involving the formation and development of white blood cells.

The identifying step involves a medical diagnosis to identify a subject that suffers from a disease or condition that would benefit from stem cell recruitment, proliferation, or differentiation. For example, it is known that myelosuppression, which is characterized by reduced white blood cell counts and may be due to reduced production of such cells from stem cells or bone marrow origin, is a serious side effect of many cancer chemotherapy drugs. Thus, in one variation, the identifying step comprises selecting a human subject undergoing antineoplastic chemotherapy. The administering of the VEGF-B product to such a subject can be performed before, during, or after a chemotherapy dosing. VEGF-B product administration contemporaneously with, or after, administering the antineoplastic chemotherapy is preferred. The VEGF-B product is preferably administered in a dosing regimen to promote myelopoiesis.

Re-establishment of a healthy white blood cell count is an important clinical consideration for patients undergoing radiation therapy. Thus, in another variation, the identifying comprises selecting a subject undergoing radiation thereapy as the candidate for VEGF-B therapy. The VEGF-B product is preferably administered contemporaneously with or after the radiation therapy.

Similarly, re-establishment of a healthy white blood cell count is critical for bone marrow transplant patents. Thus, in another variation, the identifying comprises selecting a bone marrow transplant subject as the candidate for VEGF-B therapy. The VEGF-B product is preferably administered contemporaneously with or after the bone marrow transplant. Other patient populations include individuals that are immunosuppressed for any reason, e.g., due to infection with a human immunodeficiency virus (HIV, AIDS).

Vascular endothelial growth factor B (VEGF-B) is a naturally-occurring protein in humans and other animals, encoded by a gene in the human/animal genome. VEGF-B binds to receptor VEGFR-1 and is described in greater detail below. The term “VEGF-B product” encompasses both VEGF-B polypeptide materials as described in greater detail below, and polynucleotides that encode VEGF-B polypeptides.

Thus, in one variation, the VEGF-B product comprises a VEGF-B polypeptide. For the purposes of the invention, VEGF-B (also known as VEGF-related factor (VRF)) refers to proteins having the same amino acid sequence as a naturally-occurring VEGF-B protein, and also fragments, analogs, or variants that have sequence variation, yet retain VEGFR-1 binding affinity. In one variation, the VEGF polypeptide is glycosylated. Exemplary glycosylated VEGF-B forms are described in published U.S. Patent Application No. 2002/0068694 and U.S. Patent. Nos. 5,607,918, 5,840,693, and 5,928,939, all incorporated by reference. In a preferred embodiment, the VEGF-B polypeptide has an amino acid sequence at least 85% or 90% identical to a natural human VEGF-B sequence. Still more preferred are those polypeptides that are 91, 92, 93, 94, 95, 96, 97, 98, 99, or 100% identical at the amino acid sequence level with a naturally-occurring human VEGF-B sequence. Exemplary human VEGF-β isoforms comprise the sequences set forth in SEQ ID NOS: 2 and 4, wherein secreted mature forms begin with amino acid position 1. Nucleotide and deduced amino acid sequences for VEGF-B are deposited in GenBank under Acc. No. U48801.

The sequence variation that is contemplated also can be defined in terms of the polynucleotide that encodes the VEGF-B polypeptide. For example, the VEGF-B polypeptide binds VEGFR-1 and is preferably encoded by a polynucleotide that hybridizes under stringent conditions with the complement of the polynucleotide in SEQ ID NO: 1 or 3, both of which correspond to human VEGF-B sequences. Exemplary stringent conditions are provided below.

In another variation of the invention, the VEGF-B product comprises a polynucleotide that encodes a VEGF-B polypeptide. Preferred polynucleotides also include a promoter and/or enhancer to promote expression of the encoded VEGF-B protein in target cells of the recipient organism, as well as a stop codon, a polyadenylation signal sequence, and other sequences to facilitate expression. In a preferred embodiment, the VEGF-B product comprises an expression vector containing the VEGF-B-encoding polynucleotide. Viral vectors, such as replication-deficient adenoviral and adeno-associated viral vectors, retroviruses, lentiviruses and hybrids thereof, are preferred. In this and other embodiments, other growth factor-encoding polynucleotides may also be administered or co-administered using such vectors and expression modification elements.

In preferred embodiments, the composition that comprises the VEGF-B product further comprises a pharmaceutically acceptable carrier.

Other polypeptide factors that may modulate stem cell recruitment, proliferation, and differentiation are known, and may be co-administered with VEGF-B to enhance or modulate the recruitment, proliferation, and differentiation effects of VEGF-B. Thus, in one preferred variation, the method further comprises administering to the subject a myelopoietic agent selected from the group consisting of:

(a) granulocyte colony stimulating factor (G-CSF), macrophage-CSF (M-CSF), granulocyte-macrophage-CSF (GM-CSF), interleukin-3 (IL-3), stem cell factor (SCF), vascular endothelial growth factor (VEGF or VEGF-A), vascular endothelial growth factor C (VEGF-C), vascular endothelial growth factor D (VEGF-D), vascular endothelial growth factor E (VEGF-E),placental growth factor (PlGF) platelet derived growth factor A (PDGF-A), platelet derived growth factor B (PDGF-B), platelet derived growth factor C (PDGF-C), and platelet derived growth factor D (PDGF-D), NZ2 VEGF, D1701 VEGF-like protein, NZ10 VEGF-like protein (described in International Patent Application PCT/US99/25869), and fallotein;

(b) a polynucleotide comprising a nucleotide sequence encoding any member of (a), and

(c) combinations of one or more of these polypeptides or polynucleotides.

All of these growth factors have been described in literature, including the following:

Granulocyte colony stimulating factor (G-CSF), Swiss-Prot No. P09919, Nagata, et al., “Molecular cloning and expression of cDNA for human granulocyte colony-stimulating factor,” Nature 319:415-418(1986). G-CSF Genbank Acc. No.: S69115, Shimane, et al., “Molecular Cloning and Characterization of G-CSF Induced Gene cDNA,” Biochem. Biophys. Res. Commun., 199(1):26-32 (1994).

Interleukin-3 (IL-3), Swiss-Prot No. P26951, Kitamura, et al., “Expression cloning of the human IL-3 receptor cDNA reveals a shared beta subunit for the human IL-3 and GM-CSF receptors,” Cell, 66:1165-74(1991). IL-3, Gen Bank Acc. No. M33135, Phillips, et al., “Synthesis and expression of the gene encoding human interleukin-3,” Gene, 84(2):501-507 (1989).

Macrophage-CSF (M-CSF), Swiss-Prot No. P09603, Kawasaki, et al., “Molecular cloning of a complementary DNA encoding human macrophage-specific colony-stimulating factor (CSF-1),” Science 230:291-296(1985). M-CSF Genbank Acc. No. M64592, Cerretti, et al., “Human Macrophage-Colony Stimulating Factor: Alternative RNA and Protein Processing From a Single Gene,” Mol. Immunol. 25 (8):761-770 (1988).

Stem cell factor (SCF), Swiss Prot No: P21583, Martin, et al., “Primary structure and functional expression of rat and human stem cell factor DNAs,” Cell 63:203-211(1990). SCF, Genbank Acc. No. M59964, Martin, et al., “Primary Structure and Functional Expression of Rat and Human Stem Cell Factor DNAs,” Cell 63 (1):203-211 (1990).

Granulocyte-macrophage-CSF (GM-CSF), Swiss-Prot No.: PO4141, Lee et al., “Isolation of cDNA for a human granulocyte-macrophage colony-stimulating factor by functional expression in mammalian cells,” Proc. Natl. Acad. Sci. USA 82:4360-4364(1985).

Vascular endothelial growth factor (see e.g., GenBank Acc. No. Q16889 referred to herein for clarity as VEGF-A or by particular isoform), Swiss Prot No. P15692, Leung, et al., “Vascular endothelial growth factor is a secreted angiogenic mitogen,” Science 246:1306-09(1989). VEGF clone (a 581 bp cDNA covering bps 57-638, Genbank Acc. No. 15997). VEGF-A polynucleotide and polypeptide sequences are provided in SEQ ID NOS: 11 and 12 respectively.

Vascular endothelial growth factor C (VEGF-C), Swiss-Prot No.: P49767, Joukov, et al., “A novel vascular endothelial growth factor, VEGF-C, is a ligand for the Flt4 (VEGFR-3) and KDR (VEGFR-2) receptor tyrosine kinases,” EMBO. J. 15:290-298(1996), EMBO J. 15:1751-1751(1996). VEGF-C (see e.g., GenBank Acc. No. X94216; also known as VEGF related protein (VRP)). VEGF-C cDNA insert (Genbank Acc. No. X94216), see also U.S. Pat. No. 6,361,946. VEGF-C polynucleotide and polypeptide sequences are provided in SEQ ID NOS: 13 and 14 respectively.

Vascular endothelial growth factor D (VEGF-D (also known as c-fos-induced growth factor (FIGF); see e.g., Genbank Acc. No. AJ000185)), Swiss-Prot No.: 043915, Yamada et al., “Molecular cloning of a novel vascular endothelial growth factor, VEGF-D,” Genomics 42:483-488(1997). VEGF-D, Gen Bank Acc. No. D89630, Yamada et al., “Molecular Cloning of a Novel Vascular Endothelial Growth Factor, VEGF-D,” Genomics, 42(3):483-488 (1997). VEGF-D polynucleotide and polypeptide sequences are provided in SEQ ID NOS: 17 and 18 respectively.

Vascular endothelial growth factor E (VEGF-E) (also known as NZ7 VEGF or OV NZ7; see e.g., GenBank Acc. No. S67522). VEGF-E polynucleotide and polypeptide sequences are provided in SEQ ID NOS: 19 and 20 respectively.

Placental growth factor (PlGF), Maglione et al., Proc. Natl. Acad. Sci. USA, 88(20):9267-71 (1996) (PlGF, GenBank Acc. No. X54936). PlGF polynucleotide and polypeptide sequences are provided in SEQ ID NOS: 15 and 16 respectively.

Platelet-derived growth factors such as: Platelet-derived growth factor A (PDGF-A) (see e.g., GenBank Acc. No. X06374). PDGF-A polynucleotide and polypeptide sequences are provided in SEQ ID NOS: 23 and 24 respectively. Platelet-derived growth factor B (PDGF-B) (see e.g., GenBank Acc. No. M12783). PDGF-B polynucleotide and polypeptide sequences are provided in SEQ ID NOS: 25 and 26 respectively. Platelet-derived growth factor C (PDGF-C) polynucleotide and polypeptide sequences are provided in SEQ ID NOS: 6 and 7 respectively. Platelet-derived growth factor D (PDGF-D) polynucleotide and polypeptide sequences are provided in SEQ ID NOS: 8 and 9 respectively.

Other VEGF/PDGF family members or molecules having homology thereto such as: NZ2 VEGF (also known as OV NZ2; see e.g., GenBank Acc. No. S67520). NZ2 VEGF polynucleotide and polypeptide sequences are provided in SEQ ID NOS: 21 and 22 respectively. DI 701 VEGF-like protein (see e.g., GenBank Acc. No. AF106020; Meyer et al., EMBO J. 18:363-374). D1701 VEGF-like polynucleotide and polypeptide sequences are provided in SEQ ID NOS: 29 and 30 respectively. NZ10 VEGF-like protein (described in International Patent Application PCT/US99/25869) [Stacker and Achen, Growth Factors 17:1-11 (1999); Neufeld et al., FASEB J 13:9-22 (1999); Ferrara, J Mol Med 77:527-543 (1999)]. Fallotein, disclosed in the EMBL database (Acc. No. AF091434), which has structural characteristics of the PDGF/VEGF family of growth factors. Fallotein polynucleotide and polypeptide sequences are provided in SEQ ID NOS: 27 and 28 respectively.

The above-listed growth factors are not intended to be an exhaustive list. Use of any growth factor that can stimulate stem (progenitor) cells are contemplated as part of the present invention.

For the purposes of practicing the invention, biologically active fragments of these polypeptides and variants (e.g., variants with at least 90 or 95% identity to a wildtype form or an active fragment thereof) are considered equivalents of the polypeptides themselves. The same analysis applies with respect to polynucleotides encoding such polypeptides. Compositions, including those for use in manufacturing a medicament, comprising one or more growth factor products are also contemplated. The compositions used in the methods of the present invention are themselves considered to be part of the invention.

Another embodiment of the invention is a method of stimulating stem cell proliferation or differentiation, comprising, obtaining a biological sample from a mammalian subject, wherein said sample comprises stem cells, and contacting the stem cells with a composition comprising a vascular endothelial growth factor B (VEGF-B) product. In this method, the beneficial effects of the VEGF-B are imparted to cells from a human or animal subject outside of the body of the human or other animal subject. Such therapy may be desirable to avoid VEGF-B side effects, or to prepare a treated cell sample for use in a medical procedure.

The biological sample can be any tissue or fluid sample from which stem cells are found. Blood and bone marrow are preferred sources for the biological sample, as is umbilical cord blood.

In a preferred embodiment, the biological sample is subjected to at least some purification and/or isolation procedures to purify or isolate the stem cells. For example, removal of red blood cells from a blood sample constitutes one level of purification/isolation. Still further purification, e.g., to select those nucleated cells that are CD34+ and/or VEGFR-1+, may be performed prior to the VEGF-B treatment. In a preferred embodiment, the purified stem cells comprise VEGFR-1+ or CD34+ or CD133+stem cells. Still more preferred are stem cells that comprise two or more of these markers.

Likewise, in some variations of the invention, it is desirable to purify or isolate the stem cells after the VEGF-B treatment to select those cells that have proliferated or differentiated in response to the VEGF-B treatment.

In one variation, the contacting step comprises culturing the stem cells in a culture containing the VEGF-B product. 1-10 μg protein/ml growth medium will give maximum growth stimulation. In still another variation, the contacting comprises transforming or transfecting the stem cells with a VEGF-B transgene.

In preferred variations, the method further comprises a step of returning the stem cells to the mammalian subject from which they were originally removed. Alternatively, the method comprises a step of transplanting the cells into a different mammalian subject. Human subjects are preferred. In preferred embodiments, where the cell donor is a close relative, or has a substantially identical human leukocyte antigen (HLA) profile.

Such ex vivo therapy is useful in a variety of contexts. For example, with a human subject that needs antineoplastic radiation or chemotherapy, healthy stem cells can be removed prior to the radiation or chemotherapy, cultured according to the invention, and returned following the radiation or chemotherapy. Thus, the biological sample is obtained prior to administering a dose of chemotherapy or radiation, and the stem cells are returned to the human subject after the contacting step and after the dose of chemotherapy or radiation.

The method also is useful for autologous or heterologous bone marrow transplantation. Similarly, the stem cells treated according to the method of the invention are expected to improve the success and reduce side effects of organ or tissue transplantation and graft attachments. In one variation, the cells are seeded into a tissue, organ, or artificial matrix ex vivo, and said tissue, organ, or artificial matrix is attached, implanted, or transplanted into the mammalian subject.

The term “VEGF-B product” for this embodiment of the invention has the same meaning set forth above.

The beneficial effects of contacting the cells with VEGF-B can be further enhanced by contacting the cells with one or more additional myelopoietic agents, as described above. These additional agents can be used contemporaneously with the VEGF-B product or serially, in any order.

In ex vivo embodiments, active forms of particular growth factor(s) are preferred for contacting the cells. For example, proteins that are naturally synthesized as pre-proteins, prepro-proteins, or other pre-modified forms that are not fully active are preferably administered in processed or modified forms that are active. Polynucleotides for use in ex vivo therapy are preferably manipulated to produce a “recombinantly processed” form of polypeptide by removal—at the polynucleotide level—of sequences that encode pro-peptides or other domains whose removal is required for optimal activity. Alternatively, if inactive growth factors are applied, then agents may be co-administered that result in the activation of the growth factor.

For example, a protease can be co-administered with PDGF-C in order to cleave the CUB domain and activate the protein. “Activation” is understood as a processing of a growth factor so that it is able to bind to and/or activate a receptor of the growth factor.

Another embodiment of the invention provides a method of stimulating stem cell recruitment, proliferation, or differentiation comprising, identifying a human subject in need of stem cell recruitment, proliferation, or differentiation, and administering to the human subject a composition comprising a platelet derived growth factor (PDGF) product. This embodiment and its numerous variations are similar to an embodiment described above with respect to VEGF-B, except that a PDGF product is employed in this embodiment. As described herein in detail, the biological activities of PDGFs, expecially PDGF-C, make such methods particularly useful for treating ischemic conditions.

In one variation the PDGF product comprises a PDGF polypeptide. Naturally occurring PDGF polypeptides are preferred, and human PDGF polypeptides are highly preferred. At least four distinct PDGF family members have been identified, PDGF-A, PDGF-B, PDGF-C, and PDGF-D.

PDGF-A and PDGF-B were characterized first in the literature and have thus been the subject of a greater body of research and development. Homo- and heterodimers have been formed with these polypeptides, and variants have been described with altered amino acid sequences yet the same or similar receptor binding properties. Exemplary PDGF-A and -B polypeptides for use in the invention have been described in U.S. Pat. Nos. 5,605,816 (PDGF-A and A/B heterodimers); 4,889,919 (PDGF-A homodimers); 5,759,815 (recombinant production of PDGF-A or -B in prokaryotes and formation of various dimers); 5,889,149 (PDGF-AB isoforms); 4,845,075 and 5,428,010 and 5,516,896 (PDGF-BB homodimers); 5,272,064 and 5,512,545 (PDGF-B analogues); 5,905,142 (protease-resistant PDGF-B analogues); and 5,128,321 and 5,498,600 and 5,474,982 (PDGF-A/B mosaics). In addition to the foregoing patent documents, there is substantial scientific literature describing and characterizing PDGF-A and -B proteins.

In a preferred embodiment, the PDGF polypeptide comprises a PDGF-C or PDGF-D polypeptide. PDGF-C polypeptides and polynucleotides were characterized by Eriksson et al. in International Patent Publication No. WO 00/18212, U.S. Patent Application Publication No. 2002/0164687 A1, and U.S. patent application Ser. No. 10/303,997 [published as U.S. Pat. Publ. No. 2003/0211994]. PDGF-D polynucleotides and polypeptides were characterized by Eriksson, et al. in International Patent Publication No. WO 00/27879 and U.S. Patent Application Publication No. 2002/0164710 A1. These documents are all incorporated by reference in their entirety. As described therein, PDGF-C and -D bind to PDGF receptors alpha and beta, respectively. However, a noteworthy distinction between these polypeptides and PDGF-A and -B is that PDGF-C and -D each possess an amino-terminal CUB domain that can be proteolytically cleaved to yield a biologically active (receptor binding) carboxy-terminal domain with sequence homology to other PDGF family members. For convenience, exemplary PDGF-C and -D polynucleotide and deduced amino acid sequences have been appended hereto as SEQ ID NOS: 6-9.

A preferred form of PDGF-C comprises the PDGF/VEGF homology domain (PVHD) of PDGF-C and retains receptor binding and activation functions. The minimal domain is approximately residues 230-345 of SEQ ID NO: 7. However, the domain can extend towards the N terminus up to residue 164. The PVHD of PDGF-C is also referred to as truncated PDGF-C. The truncated PDGF-C is an activated form of PDGF-C. A putative proteolytic site in PDGF-C is found in residues 231-234 of SEQ ID NO: 7, a dibasic motif. The putative proteolytic site is also found in PDGF-A, PDGF-B, VEGF-C and VEGF-D. In these four proteins, the putative proteolytic site is also found just before the minimal domain for the PDGF/VEGF homology domain. The CUB domain of PDGF-C represents approximately amino acid residues 23-159 of SEQ ID NO: 7. U.S. Patent Application Publication No.: 2002/0164687.

Similar to PDGF-C, PDGF-D has a two domain structure with a N-terminal CUB domain (described as approximately residues 67-167 or 54-171 of SEQ ID NO: 9) and a C-terminal PDGF/VEGF homology domain (PVHD). A putative proteolytic site in PDGF-D is found in residues 255-258 of SEQ ID NO: 9. A preferred PDGF-D polypeptide comprises the PDGF/VEGF homology domain (PVHD) of PDGF-D and retains receptor binding and activation functions. The minimal domain of PDGF-D is approximately residues 272-362 or 255-370 of SEQ ID NO: 9. However, PDGF-D's PVHD extends toward the N terminus up to residue 235 of SEQ ID NO: 9. The truncated PDGF-D is the putative activated form of PDGF-D. U.S. Patent Application Publication No. 2002/0164710.

As discussed elsewhere herein in detail, members of the PDGF/VEGF family form homodimers (and sometimes heterodimers). References herein to specific dimeric forms (e.g., PDGF-CC for PDGF-C homodimers) is sometimes made for context or clarity. References to polypeptide forms (e.g. PDGF-C) are not meant to imply anything about the monomeric or dimeric or other forms of the polypeptide composition, unless specifically stated.

In addition to naturally occurring PDGF polypeptides, variant forms that still bind to and/or the respective PDGF receptors (including receptor homodimers and heterodimers) also may be used in the invention described herein. Variants with at least 90% amino acid sequence identity to a naturally occurring human PDGF-A -B, -C, or -D polypeptide are preferred. Still more preferred is at least 92%, 94%, 95%, 96%, 97%, 98%, or 99% sequence identity. Thus, in another variation, the PDGF polypeptide comprises a portion of the amino acid sequence set forth in SEQ ID NOS: 24, 26, 7, or 9 that is effective to bind PDGFR-α and/or PDGFR-β. In still another variation, the PDGF polypeptide binds PDGFR-α and/or PDGFR-β and is encoded by a polynucleotide that hybridizes under stringent conditions with the complement of the polynucleotide in SEQ ID NO: 23, 25, 6, or 8.

In some embodiments, the PDGF-C polypeptide has at least 90%, 92%, 94%, 95%, 96%, 97%, 98%, or 99% sequence identity to residues 230-345 of SEQ ID NO: 7. In some embodiments, the PDGF-D polypeptide has at least 90%, 92%, 94%, 95%, 96%, 97%, 98%, or 99% sequence identity to residues 272-362 of SEQ ID NO: 9.

In another variation, the PDGF product comprises a polynucleotide that encodes a PDGF polypeptide. Expression of the polynucleotide in or near target progenitor cells results in production of effective quantities of PDGF polypeptides. Thus, in preferred variations, the PDGF product comprises a vector, such as a viral vector, containing the polynucleotide. Exemplary vectors include replication-deficient adenoviral or adeno-associated viral vectors, as well as retroviruses and lentiviruses. However, any vector effective for delivery of a PDGF polynucleotide to target cells is contemplated.

In still another variation, the PDGF product is co-administered with one or more additional myelopoietic agents which together stimulate recruitment, proliferation, and/or differentiation of the target cells in a desirable way. Exemplary agents for coadministration with a PDGF product include those growth factors and other agents described earlier in respect to VEGF-B therapies, as is coadministration with VEGF-B. Such growth factors include: (a) granulocyte colony stimulating factor (G-CSF), macrophage-CSF (M-CSF), granulocyte-macrophage-CSF (GM-CSF), interleukin-3 (IL-3), stem cell factor (SCF), vascular endothelial growth factor (VEGF), vascular endothelial growth factor B (VEGF-B) vascular endothelial growth factor C (VEGF-C), vascular endothelial growth factor D (VEGF-D), and placental growth factor (PlGF); (b) a polynucleotide comprising a nucleotide sequence encoding any member of (a), and (c) combinations thereof. Other growth factors not listed may also be employed.

The present invention is also based on the discovery that PDGF-CC (a PDGF-C dimer) enhances post-ischemic revascularization in the heart and limb, apparently exerting effects on vascular progenitor and mature cells of both endothelial and smooth muscle cell/fibroblast lineages. Revascularization of brain, heart, limb, and other tissues that have become ischemic are contemplated. As described herein in detail, evidence indicates that PDGF-CC mobilizes endothelial progenitor cells, induces differentiation of bone marrow cells into endothelial cells, stimulates migration of endothelial cells, and upregulates VEGF expression. Moreover, PDGF-CC induces the differentiation of bone marrow cells into smooth muscle cells (SMC) and stimulates SMC growth and migration during vessel sprouting. This pleiotropic activity of PDGF-CC on vascular progenitors and differentiated cells of both endothelial and smooth muscle cell/fibroblast lineages together with evidence of its safety profile (lack of hemangioma-genesis, edema or fibrosis), and evidence that certain activity is restricted to ischemic conditions, provides novel therapeutic indications for PDGF-C products in vivo and ex vivo for treating ischemic diseases.

One embodiment includes a method of stimulating stem cell proliferation or differentiation. A biological sample comprising stems cells is obtained from a mammalian subject, wherein said sample comprises stem cells. The stem cells are then contacted with a composition comprising a platelet derived growth factor-C (PDGF-C) product. In one variation stem cells are isolated from the biological sample prior to the contacting step, including variation wherein AC133+/CD34+ cells are isolated from the biological sample. In some variations, stems cells are contacted with the PDGF-C product until particular cell surface markers appear (become more prominent in) and/or particular markers disappear from (become less prominent in) a stem cell population. In one variation, the contacting continues until stem cells differentiate into CD144+cells, at which time PDGF-C treatment is stopped. In another variation, the contacting continues until stem cells differentiate into SMA+/CD 144−/CD31−/CD34− cells. In some embodiments, a VEGF-A product in addition to a PDGF-C product is used to contact the cells.

The PDGF-C products contemplated from practice of the foregoing method include PDGF-C polypeptides and polynucleotides that encode them, or combination thereof. Where the PDGF-C product comprises a PDGF-C polypeptide, the PDGF-C polypeptide preferably comprises an amino acid sequence at least 95% identical to the amino acid sequence of SEQ ID NO: 10 and binds to either a PDGFR-α/α homodimer or PDGFR-α/β heterodimer receptor. In one embodiment, the polypeptide is present in the composition as homodimers (PDGF-CC). In some variations, the PDGF-C polypeptide is encoded by a polynucleotide that hybridizes under stringent conditions with the complement of the polynucleotide in SEQ ID NO: 6.

One embodiment of ex vivo therapy of the invention involves separate treatment of two or more aliquots of progenitor cells from a patient with two or more distinct growth factor regimens of one or more growth factors per regimen. In this way, differentiation into two or more distinct populations of cells is achieved. These distinct cell populations preferably complement each other in vivo to achieve improved therapeutic benefit when readministered to a patient. In one embodiment, stem cell proliferation or differentiation is carried out by first obtaining a biological sample from a mammalian subject, wherein said sample comprises stem cells. A first aliquot of the stem cells is contacted with a first composition comprising a first growth factor product selected from a VEGF-B product and PDGF-C product. A second aliquot of the stem cells with a second composition comprising a second growth factor product independently selected from the group consisting of VEGF-A, VEGF-B, VEGF-C, VEGF-D, PDGF-A, PDGF-B, PDGF-C, and PlGF products. In such an embodiment, the first and second growth factor products are generally not the same. In some variations, the first growth factor product is a PDGF-C product and the second growth factor product is a VEGF-A product.

In another embodiment, the differentiation of stem cells into both endothelial and smooth muscle cells is promoted by obtaining a biological sample comprising stem cells from a mammalian subject, wherein said sample comprises stem cells. The obtained cells are then contacted with a composition comprising a platelet-derived growth factor-C (PDGF-C) product, in an amount and for a time sufficient to cause the cells to differentiate into both endothelial and smooth muscle cells. In some variations, the contacted cells are returned to the mammalian subject. In some variations, the mammalian subject who receives the cells has an ischemic condition, including one affecting tissue of the heart (e.g., infarction), brain (e.g., stroke) or limb (e.g., peripheral clot).

In another embodiment, an ischemic condition is ameliorated by first (optionally) diagnosing a mammalian subject with an ischemic condition. A biological sample comprising stem cells is obtained from a subject so diagnosed. The obtained cells are contacted with a composition comprising a platelet-derived growth factor-C (PDGF-C) product, in an amount and for a time sufficient to cause the cells to differentiate into both endothelial and smooth muscle cells. The contacted cells are then returned to the mammalian subject. In some embodiments, the cells are returned by implanting or injecting the cells into ischemic tissue of the mammalian subject.

Still another embodiment of the invention is a method of stimulating stem cell proliferation or differentiation, comprising obtaining a biological sample from a mammalian subject, wherein said sample comprises stem cells, and contacting the stem cells with a composition comprising a platelet derived growth factor (PDGF) product. This method and its variations are similar to an embodiment summarized above with respect to VEGF-B. In a preferred variation, the contacting comprises culturing the stem cells in a culture containing the PDGF product.

In one variation, the stem cells are purified and isolated after obtaining the sample and before contacting them with the PDGF product. In one variation, the stem cells are purified and isolated after treatment with the PDGF product in the contacting step. Preferred populations of stem cells for purification include those expressing one or more of the following receptors/markers on their cell surface: PDGFR-alpha, PDGFR-beta, and CD34.

Progenitor/stem cells that have been prepared according to the various ex vivo embodiments of the invention are useful in a number of therapeutic contexts when returned to the host from which the sample was originally obtained, or transplanted into a different host. The cells can be returned into the bloodstream or bone marrow intravenously or by injection, or alternatively, seeded into a tissue, organ, or artificial matrix ex vivo, and said tissue, organ, or artificial matrix is attached, implanted, or transplanted into the mammalian subject.

In one preferred embodiment, the cells are used to treat a human subject that needs antineoplastic chemotherapy. For example, the biological sample is obtained prior to administering a dose of chemotherapy, and the stem cells are returned to the human subject after the contacting and after the dose of chemotherapy.

In one preferred embodiment, the cells are used to treat a human subject who has been diagnosed with a cardiovascular diseases, including diabetes-related vascular complications. Human subjects suffereing from either Type I (insulin-dependent) or Type II (non-insulin-dependent) diabetes mellitus are contemplated, as are non-diabetic human subjects who suffer from cardiovascular diseases. Millions of patients suffer from insufficient blood supply to tissues, particularly to the heart, brain and legs. The growing population of diabetics is particularly prone to developing these life-threatening conditions, as are those of risk of heart attacks and strokes. Therapeutic angio/arteriogenic factors are therefore of interest for alleviating such complications by inducing new blood vessels. The building of new stable and functional vessels relies on a concerted action of vascular progenitors and differentiated endothelial and smooth muscle cells. Therapeutic angiogenesis may thus require co-administration of factors that affect both lineages. Alternatively, molecules with pleiotropic effects on both lineages would be attractive, but only a few have been identified thus far.

In other therapeutic contexts, it may be desirable to suppress progenitor/stem cell recruitment, proliferation, or differentiation. Further embodiments of the invention are methods of inhibiting/suppressing progenitor/stem cell recruitment, proliferation, or differentiation by contacting the cells (in vivo or ex vivo) with inhibitors specific for the VEGF-B or the PDGF products described above. Exemplary inhibitors, including antibodies, antisense molecules, and aptamers, are described in greater detail below.

It will be apparent that many aspects of the invention relate to new uses of various polynucleotide and protein products. In still another variation, the invention provides for the use of any of the aforementioned products in the manufacture of a medicament for stimulating stem cell recruitment, proliferation, and/or differentiation, or a medicament for treatment of any disease or condition that would benefit from stem cell recruitment, proliferation, and/or differentiation.

Likewise, the specific inhibitors described above are useful in the manufacture of a medicament for inhibition of stem cell recruitment, proliferation, and/or differentiation, or a medicament for treatment of any disease or condition that would benefit (even transiently) from inhibition of stem cell recruitment, proliferation, and/or differentiation.

Unitary activity on a single type of cell leading to nonfunctional capillaries, or harmful side effects involving edema or angioma-genesis, is often the central problem for therapeutic vasculogenic, angio/arteriogenic, and neoangiogenic agents under trial. Molecules with pleiotropic activities affecting multiple vascular cells or stages of vasculogenesis, angio/arteriogenesis, and neoangiogenesis, but with minimal side effects, thus become attractive means to treat tissue ischemia. There are considerable potential advantages of choosing such molecules, including mobilizing multiple vascular cells and molecules needed to build functional vessels by a single delivery of one effector molecule, and the simultaneous regulation of the complex cascade of vasculogenesis, angio/arteriogenesis, and/or neoangiogenesis with one therapeutic intervention. This represents a promising paradigm of new therapeutic agents to cultivate functional vessels with more physiological functional properties in treating tissue ischemia.

Additional features and variations of the invention will be apparent to those skilled in the art from the entirety of this application, including the detailed description, and all such features are intended as aspects of the invention.

Likewise, features of the invention described herein can be re-combined into additional embodiments that also are intended as aspects of the invention, irrespective of whether the combination of features is specifically mentioned above as an aspect or embodiment of the invention. Also, only those limitations that are described herein as critical to the invention should be viewed as such; variations of the invention lacking features that have not been described herein as critical are intended as aspects of the invention.

With respect to aspects of the invention that have been described as a set or genus, every individual member of the set or genus is intended, individually, as an aspect of the invention.

In addition to the foregoing, the invention includes, as an additional aspect, all embodiments of the invention narrower in scope in any way than the variations specifically mentioned above. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1a shows VEGFR-3⁺ cells (%) in blood of 5-fluorouracil treated FVB mice (n=4), relative to total white blood cells.

FIG. 1b shows VEGFR-3⁺ (%) cells in bone marrow after 5-FU treatment of FVB mice.

FIG. 2a shows white blood cell counts of blood in 5-FU and adenovirus treated mice.

FIG. 2b shows white blood cell counts of blood in 5-FU and adenovirus treated NMRI mice.

FIG. 2c shows white blood cell counts of blood after 5-FU and adenoviral treatment on day 10 in FVB mice.

FIG. 3a shows VEGFR-1⁺ cells (%) of white blood cells in nude mice treated with an adenovirus containing a LacZ, VEGF-C, VEGF-C156S or VEGF-B transgene.

FIG. 3b shows VEGFR-2⁺ cells (%) of white blood cells in nude mice treated with an adenovirus containing a LacZ, VEGF-C, VEGF-C 156S or VEGF-B transgene.

FIG. 3c shows VEGFR-3⁺ cells (%) of white blood cells in nude mice treated with an adenovirus containing a LacZ, VEGF-C, VEGF-C156S or VEGF-B transgene.

FIG. 3d shows CD34⁺ cells (%) of white blood cells in nude mice treated with an adenovirus containing a LacZ, VEGF-C, VEGF-CI 56S or VEGF-B transgene.

FIG. 4 shows the number of endothelial progenitor cells per square millimeter in mice either treated with a control or PDGF-CC. There were three sets of control and experimental animals: non-ischemic mice, ischemic mice sacrificed after two days and ischemic mice sacrificed after five days.

FIG. 5 shows results of bone marrow cell adherence assays of control cells, cells treated with VEGF and cells treated with PDGF-CC.

FIG. 6a shows migration assay results of three types of cells—bovine aortic endothelial cells (BAEC), human microvascular endothelial cells (HMVEC) and smooth vessel cells (SMC)—and how that migration was influenced by the absence or presence of various growth factors—VEGF, PDGF-AA, PDGF-BB, and PDGF-CC.

FIG. 6b shows proliferation assay results of HMVEC influenced by the absence or presence of various growth factors—VEGF, PDGF-AA, PDGF-BB, and PDGF-CC.

FIG. 7a shows the results from aortic ring assays, and specifically microvessel outgrowth influenced by the absence or presence of various growth factors—VEGF, PDGF-AA, PDGF-BB, and PDGF-CC.

FIG. 7b shows the results from aortic ring assays, and specifically fibroblast proliferation and migration influenced by the absence or presence of various growth factors—VEGF, PDGF-AA, PDGF-BB, and PDGF-CC.

FIG. 8a shows in an upper panel a Western blot of protein immunoprecipitated from lysates of human smooth muscle cells (hSMC) and NIH-3T3 cells (a fibroblast cell line) using an anti-PDGFR-α antibody. In a lower panel a Western blot of proteins from human smooth muscle cells (hSMC) and NIH-3T3 cells using an anti-phosphotyrosine antibody is shown.

FIG. 8b shows proliferation of NIH-3T3 cells and hSMC cells influenced by various PDGF homodimers.

FIG. 8c shows on the left-hand side the results from both RNA protection assays (RPA) (top panel) and a Western blot (bottom panel) of either control (vector) NIH-3T3 cells or NIH-3T3 cells overexpressing PDGF-C. The right hand shows the results of ELISAs of either control (vector) NIH-3T3 cells or NIH-3T3 cells overexpressing PDGF-C.

### DETAILED DESCRIPTION

The term “VEGF-B” as used in the present invention encompasses those polypeptides identified as VEGF-B in U.S. Pat. No. 6,331,301, which is incorporated herein in its entirety, as well as published U.S. Application No. 2003/0008824.

VEGF-B comprises, but is not limited to, both the VEGF-B₁₆₇ and/or VEGF-B₁₈₆ isoforms or a fragment or analog thereof having the ability to bind VEGFR-1. Active analogs should exhibit at least 85% sequence identity, preferably at least 90% sequence identity, particularly preferably at least 95% sequence identity, and especially preferably at least 98% sequence identity to the natural VEGF-B polypeptides, as determined by BLAST analysis. The active substance typically will include the amino acid sequence Pro-Xaa-Cys-Val-Xaa-Xaa-Xaa-Arg-Cys-Xaa-Gly-Cys-Cys (where Xaa may be any amino acid) (SEQ ID NO: 5) that is characteristic of VEGF-B.

Use of polypeptides comprising VEGF-B sequences modified with conservative substitutions, insertions, and/or deletions, but which still retain the biological activity of VEGF-B is within the scope of the invention. Standard methods can readily be used to generate such polypeptides including site-directed mutagenesis of VEGF-B polynucleotides, or specific enzymatic cleavage and ligation. Similarly, use of peptidomimetic compounds or compounds in which one or more amino acid residues are replaced by a non-naturally-occurring amino acid or an amino acid analog that retains the required aspects of the biological activity of VEGF-B is contemplated.

The term PDGF comprises, but is not limited to PDGF-A, PDGF-B, PDGF-C, and PDGF-D, or a fragment or analog thereof having the ability to bind PDGF-receptors. PDGF-A may bind to and/or activate PDGFR-α/α homodimers. PDGF-β may bind to and/or activate PDGFR-α/α homodimers, PDGFR-α/β heterodimers and PDGR-β/β homodimers. PDGF-C may bind to and/or activate PDGFR-α/α homodimers. PDGF-C may also bind to and/or activate PDGFR-α/β heterodimers via PDGFR-α binding. PDGF-D may bind to and/or activate PDGFR-β/β homodimers. PDGF-D may also bind to and/or activate PDGFR-α/β heterodimers via PDGFR-β binding. Active analogs should exhibit at least 85% sequence identity, preferably at least 90% sequence identity, particularly preferable at least 95% sequence identity, and especially preferable at least 98% sequence identity to the natural PDGF polypeptides, as determined by BLAST analysis.

Use of polypeptides comprising PDGF sequences modified with conservative substitutions, insertions, and/or deletions, but which still retain the biological activity of PDGFs is within the scope of the invention. Standard methods can readily be used to generate such polypeptides including site-directed mutagenesis of PDGF polynucleotides, or specific enzymatic cleavage and ligation. Similarly, use of peptidomimetic compounds or compounds in which one or more amino acid residues are replaced by a non-naturally-occurring amino acid or an amino acid analog that retains the required aspects of the biological activity of PDGFs is contemplated.

In addition, variant forms of VEGF-B or PDGF polypeptides that may result from alternative splicing and naturally-occurring allelic variation of the nucleic acid sequence encoding VEGF-B or a PDGF are useful in the invention. Allelic variants are well known in the art, and represent alternative forms or a nucleic acid sequence that comprise substitution, deletion or addition of one or more nucleotides, but which do not result in any substantial functional alteration of the encoded polypeptide.

Variant forms of VEGF-B or a PDGF can be prepared by targeting non-essential regions of a VEGF-B or PDGF polypeptide for modification. These non-essential regions are expected to fall outside the strongly-conserved regions of the VEGF/PDGF family of growth factors. In particular, the growth factors of the PDGF/VEGF family, including VEGF-B and the PDGFs, are dimeric, and at least VEGF-A, VEGF-B, VEGF-C, VEGF-D, PlGF, PDGF-A and PDGF-B show complete conservation of eight cysteine residues in the N-terminal domains, i.e. the PDGF/VEGF-like domains. [Olofsson, et al., Proc. Nat'l. Acad. Sci. USA, 93:2576-2581 (1996); Joukov, et al., EMBO J., 15:290-298 (1996).] These cysteines are thought to be involved in intra- and inter-molecular disulfide bonding. In addition there are further strongly, but not completely, conserved cysteine residues in the C-terminal domains. Loops 1, 2 and 3 of each subunit, which are formed by intra-molecular disulfide bonding, are involved in binding to the receptors for the PDGF/VEGF family of growth factors. [Andersson, et al., Growth Factors, 12:159-64 (1995).]

These conserved cysteine residues are preferably preserved in any proposed variant form, although there may be exceptions, because receptor-binding VEGF-B analogs are known in which one or more of the cysteines is not conserved. Similarly, the active sites present in loops 1, 2 and 3 also should be preserved. Other regions of the molecule can be expected to be of lesser importance for biological function, and therefore offer suitable targets for modification. Modified polypeptides can readily be tested for their ability to show the biological activity of VEGF-B or a PDGF by routine activity assay procedures such as a VEGFR-1 binding assay or a stem cell proliferation assay based on the examples set forth below.

Preferably, where amino acid substitution is used, the substitution is conservative, i.e. an amino acid is replaced by one of similar size and with similar charge properties.

As used herein, the term “conservative substitution” denotes the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids that can be substituted for one another include asparagine, glutamine, serine and threonine. The term “conservative substitution” also includes the use of a substituted amino acid in place of an unsubstituted parent amino acid.

Alternatively, conservative amino acids can be grouped as described in Lehninger, (Biochemistry, Second Edition; Worth Publishers, Inc. NY:NY, pp. 71-77 (1975)) as set out in the following: Non-polar (hydrophobic) A. Aliphatic: A, L, I, V, P, B. Aromatic: F, W, C. Sulfur-containing: M, D. Borderline: G. Uncharged-polar A. Hydroxyl: S, T, Y, B. Amides: N, Q, C. Sulfhydryl: C, D. Borderline: G. Positively Charged (Basic): K, R, H. Negatively Charged (Acidic): D, E.

VEGF-B or a PDGF protein can be modified, for instance, by glycosylation, amidation, carboxylation, or phosphorylation, or by the creation of acid addition salts, amides, esters, in particular C-terminal esters, and N-acyl derivatives. The proteins also can be modified to create peptide derivatives by forming covalent or noncovalent complexes with other moieties. Covalently bound complexes can be prepared by linking the chemical moieties to functional groups on the side chains of amino acids comprising the peptides, or at the N- or C-terminus.

VEGF-B and PDGF proteins can be conjugated to a reporter group, including, but not limited to a radiolabel, a fluorescent label, an enzyme (e.g., that catalyzes a calorimetric or fluorometric reaction), a substrate, a solid matrix, or a carrier (e.g., biotin or avidin).

Examples of VEGF-B analogs are described in WO 98/28621 and in Olofsson, et al., Proc. Nat'l. Acad. Sci. USA, 95:11709-11714 (1998), both incorporated herein by reference. Examples of PDGF analogs are described in U.S. Pat. Nos. 5,512,545, and 5,474,982; U.S. Patent Application Nos.: 20020164687 and 20020164710.

VEGF-B and PDGF polypeptides are preferably produced by expression of DNA sequences that encode them such as DNAs that correspond to, or that hybridize under stringent conditions with the compliments of SEQ ID NOS: 1 and 3. Suitable hybridization conditions include, for example, 50% formamide, 5×SSPE buffer, 5× Denhardts solution, 0.5% SDS and 100 μg/ml of salmon sperm DNA at 42° C. overnight, followed by washing 2×30 minutes in 2×SSC at 55° C. Such hybridization conditions are applicable to any polynucleotide encoding one or more of the growth factors of the present invention.

The invention is also directed to an isolated and/or purified DNA that corresponds to, or that hybridizes under stringent conditions with, any one of the foregoing DNA sequences.

The VEGF-B proteins and polypeptides for use in the present invention are characterized by the amino acid sequence Pro-Xaa-Cys-Val-Xaa-Xaa-Xaa-Arg-Cys-Xaa-Gly-Cys-Cys (SEQ ID NO: 5) and having the property of stimulating the recruitment, mobilization or proliferation of stem cells, including hematopoietic progenitor cells and endothelial progenitor cells, wherein the protein comprises a sequence of amino acids substantially corresponding to an amino acid sequence selected from the group consisting of the amino acid sequence of SEQ ID NOS: 2 and 4. VEGF-B dimmers may comprise VEGF-B polypeptides of identical sequence, of different VEGF-β isoforms, or other heterogeneous VEGF-B molecules.

The VEGF-B for use according to the present invention can be used in the form of a protein dimer comprising VEGF-B protein, particularly a disulfide-linked dimer. The protein dimers of the invention include both homodimers of VEGF-B and heterodimers of VEGF-B and VEGF polypeptides, as well as other VEGF family growth factors including, but not limited to placental growth factor (PlGF), which are capable of binding to VEGFR-1 (fit-1). The VEGF-B of the present invention also includes VEGF-B polypeptides that have been engineered to contain a N-glycosylation cite such as those described in Jeltsch, et al., WO 02/07514, which is incorporated herein in its entirety.

As used herein, the term “biologically active,” when used in conjunction with VEGF-B refers to a VEGF-B polypeptide that binds VEGFR-1 (also known as flt-1) in a manner substantially similar to that of full length VEGF-B, and/or that stimulates migration, proliferation and/or differentiation of a population of mammalian stem cells. As used herein, the term biologically active,” when used in conjunction with PDGF refers to a PDGF polypeptide that binds to its natural PDGF-receptor (PDGF-α and/or PDGF-β as described above) in a manner substantially similar to that of the native PDGF, and/or that stimulates migration, proliferation and/or differentiation of a population of a mammalian stem cells.

The term “vector” refers to a nucleic acid molecule amplification, replication, and/or expression vehicle in the form of a plasmid or viral DNA system where the plasmid or viral DNA may be functional with bacterial, yeast, invertebrate, and/or mammalian host cells. The vector may remain independent of host cell genomic DNA or may integrate in whole or in part with the genomic DNA. The vector will contain all necessary elements so as to be functional in any host cell it is compatible with. Such elements are set forth below.

Preparation of VEGF-B is discussed in U.S. Pat. No. 6,331,301, which is incorporated herein in its entirety.

Preparation of DNA Encoding VEGF-B or PDGF Polypeptides

A nucleic acid molecule encoding VEGF-B or PDGF can readily be obtained in a variety of ways, including, without limitation, chemical synthesis, cDNA or genomic library screening, expression library screening, and/or PCR amplification of cDNA. These methods and others useful for isolating such DNA are set forth, for example, by Sambrook, et al., “Molecular Cloning: A Laboratory Manual,” Cold Spring Harbor Laboratory Press, Cold Spring Harbor, N.Y. (1989), by Ausubel, et al., eds., “Current Protocols In Molecular Biology,” Current Protocols Press (1994), and by Berger and Kimmel, “Methods In Enzymology: Guide To Molecular Cloning Techniques,” vol. 152, Academic Press, Inc., San Diego, Calif. (1987). Preferred nucleic acid sequences encoding VEGF-B or PDGF are mammalian sequences. Most preferred nucleic acid sequences encoding VEGF-B or PDGF are human, rat, and mouse.

Chemical synthesis of a VEGF-B or PDGF nucleic acid molecule can be accomplished using methods well known in the art, such as those set forth by Engels, et al., Angew. Chem. Intl. Ed., 28:716-734 (1989). These methods include, inter alia, the phosphotriester, phosphoramidite and H-phosphonate methods of nucleic acid synthesis. Typically, the nucleic acid molecule encoding the full length VEGF-B polypeptide will be several hundred base pairs (bp) or nucleotides in length. Nucleic acids larger than about 100 nucleotides in length can be synthesized as several fragments, each fragment being up to about 100 nucleotides in length. The fragments can then be ligated together, as described below, to form a full length nucleic acid encoding the VEGF-B or PDGF polypeptide. A preferred method is polymer-supported synthesis using standard phosphoramidite chemistry.

Alternatively, the nucleic acid encoding a VEGF-B or PDGF polypeptide may be obtained by screening an appropriate cDNA library prepared from one or more tissue source(s) that express the polypeptide, or a genomic library from any subspecies. The source of the genomic library may be any tissue or tissues from any mammalian or other species believed to harbor a gene encoding VEGF-B or a VEGF-B homologue or PDGF or PDGF homologue.

The library can be screened for the presence of the VEGF-B cDNA/gene using one or more nucleic acid probes (oligonucleotides, cDNA or genomic DNA fragments that possess an acceptable level of homology to the VEGF-B or VEGF-B homologue cDNA or gene to be cloned) that will hybridize selectively with VEGF-B or VEGF-B homologue cDNA(s) or gene(s) that is(are) present in the library. The probes preferably are complementary to or encode a small region of the VEGF-B DNA sequence from the same or a similar species as the species from which the library was prepared. Alternatively, the probes may be degenerate, as discussed below.

The library also can be screened for the presence of the PDGF cDNA/gene using one or more nucleic acid probes (oligonucleotides, cDNA or genomic DNA fragments that possess an acceptable level of homology to the PDGF homologue cDNA or gene to be cloned) that will hybridize selectively with PDGF or PDGF homologue cDNA(s) or gene(s) that is(are) present in the library. The probes preferably are complementary to or encode a small region of the PDGF DNA sequence from the same or a similar species as the species from which the library was prepared. Alternatively, the probes may be degenerate, as discussed below.

Where DNA fragments (such as cDNAs) are used as probes, typical hybridization conditions are those for example as set forth in Ausubel, et al., eds., supra. After hybridization, the blot containing the library is washed at a suitable stringency, depending on several factors such as probe size, expected homology of probe to clone, type of library being screened, number of clones being screened, and the like. Examples of stringent washing solutions (which are usually low in ionic strength and are used at relatively high temperatures) are as follows. One such stringent wash is 0.015 M NaCl, 0.005 M NaCitrate and 0.1 percent SDS at 55-65° C. Another such stringent buffer is 1 mM Na₂ EDTA, 40 mM NaHPO₄, pH 7.2, and 1 percent SDS at about 40-50° C. One other stringent wash is 0.2.×SSC and 0.1 percent SDS at about 50-65° C. Such hybridization conditions are applicable to any polynucleotide encoding one or more of the growth factors of the present invention.

Another suitable method for obtaining a nucleic acid encoding a VEGF-B or PDGF polypeptide is the polymerase chain reaction (PCR). In this method, poly(A)+RNA or total RNA is extracted from a tissue that expresses VEGF-B or PDGF (such as lymphoid tissue). cDNA is then prepared from the RNA using the enzyme reverse transcriptase. Two primers typically complementary to two separate regions of the VEGF-B cDNA or PDGF cDNA (oligonucleotides) are then added to the cDNA along with a polymerase such as Taq polymerase, and the polymerase amplifies the cDNA region between the two primers.

Preparation of a Vector for VEGF-B or PDGF Expression

After cloning, the cDNA or gene encoding a VEGF-B or PDGF polypeptide or fragment thereof has been isolated, it is preferably inserted into an amplification and/or expression vector in order to increase the copy number of the gene and/or to express the polypeptide in a suitable host cell and/or to transform cells in a target organism (to express VEGF-B or PDGF in vivo). Numerous commercially available vectors are suitable, though “custom made” vectors may be used as well. The vector is selected to be functional in a particular host cell or host tissue (i.e., the vector is compatible with the host cell machinery such that amplification of the VEGF-B or PDGF gene and/or expression of the gene can occur). The VEGF-B or PDGF polypeptide or fragment thereof may be amplified/expressed in prokaryotic, yeast, insect (baculovirus systems) and/or eukaryotic host cells. Selection of the host cell will depend at least in part on whether the VEGF-B or PDGF polypeptide or fragment thereof is to be glycosylated. If so, yeast, insect, or mammalian host cells are preferable; yeast cells will glycosylate the polypeptide if a glycosylation site is present on the VEGF-B or PDGF amino acid sequence.

Typically, the vectors used in any of the host cells will contain 5′ flanking sequence and other regulatory elements as well such as an enhancer(s), an origin of replication element, a transcriptional termination element, a complete intron sequence containing a donor and acceptor splice site, a signal peptide sequence, a ribosome binding site element, a polyadenylation sequence, a polylinker region for inserting the nucleic acid encoding the polypeptide to be expressed, and a selectable marker element. Optionally, the vector may contain a “tag” sequence, i.e., an oligonucleotide sequence located at the 5′ or 3′ end of the VEGF-B or PDGF coding sequence that encodes polyHis (such as hexaHis) or another small immunogenic sequence. This tag will be expressed along with the protein, and can serve as an affinity tag for purification of the VEGF-B or PDGF polypeptide from the host cell. Optionally, the tag can subsequently be removed from the purified VEGF-B or PDGF polypeptide by various means such as using a selected peptidase for example.

The vector/expression construct may optionally contain elements such as a 5′ flanking sequence, an origin of replication, a transcription termination sequence, a selectable marker sequence, a ribosome binding site, a signal sequence, and one or more intron sequences. The 5′ flanking sequence may be homologous (i.e., from the same species and/or strain as the host cell), heterologous (i.e., from a species other than the host cell species or strain), hybrid (i.e., a combination of p5′ flanking sequences from more than one source), synthetic, or it may be the native VEGF-B or PDGF 5′ flanking sequence. As such, the source of the 5′ flanking sequence may be any unicellular prokaryotic or eukaryotic organism, any vertebrate or invertebrate organism, or any plant, provided that the 5′ flanking sequence is functional in, and can be activated by, the host cell machinery.

An origin of replication is typically a part of commercial prokaryotic expression vectors, and aids in the amplification of the vector in a host cell. Amplification of the vector to a certain copy number can, in some cases, be important for optimal expression of the VEGF-B or PDGF polypeptide. If the vector of choice does not contain an origin of replication site, one may be chemically synthesized based on a known sequence, and ligated into the vector.

A transcription termination element is typically located 3′ to the end of the VEGF-B or PDGF polypeptide coding sequence and serves to terminate transcription of the VEGF-B or PDGF polypeptide. Usually, the transcription termination element in prokaryotic cells is a G-C rich fragment followed by a poly T sequence. Such elements can be cloned from a library, purchased commercially as part of a vector, and readily synthesized.

Selectable marker genes encode proteins necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media.

A ribosome binding element, commonly called the Shine-Dalgarno sequence (prokaryotes) or the Kozak sequence (eukaryotes), is necessary for translation initiation of mRNA. The element is typically located 3′ to the promoter and 5′ to the coding sequence of the polypeptide to be synthesized. The Shine-Dalgarno sequence is varied but is typically a polypurine (i.e., having a high A-G content). Many Shine-Dalgarno sequences have been identified, each of which can be readily synthesized using methods set forth above.

All of the elements set forth above, as well as others useful in this invention, are well known to the skilled artisan and are described, for example, in Sambrook, et al., “Molecular Cloning: A Laboratory Manual,” Cold Spring Harbor Laboratory Press, Cold Spring Harbor, N.Y. (1989) and Berger, et al., eds., “Guide To Molecular Cloning Techniques,” Academic Press, Inc., San Diego, Calif. (1987].

For those embodiments of the invention where the recombinant VEGF-B or PDGF is to be secreted, a signal sequence is preferably included to direct secretion from the cell where it is synthesized. Typically, the signal sequence is positioned in the coding region of the transgene towards or at the 5′ end of the coding region. Many signal sequences have been identified, and any of them that are functional in the transgenic tissue may be used in conjunction with the transgene. Therefore, the signal sequence may be homologous or heterologous to the transgene, and may be homologous or heterologous to the transgenic mammal. Additionally, the signal sequence may be chemically synthesized using methods set forth above. However, for purposes herein, preferred signal sequences are those that occur naturally with the transgene (i.e., are homologous to the transgene).

In many cases, gene transcription is increased by the presence of one or more introns on the vector. The intron may be naturally-occurring within the transgene sequence, especially where the transgene is a full length or a fragment of a genomic DNA sequence. Where the intron is not naturally-occurring within the DNA sequence (as for most cDNAs), the intron(s) may be obtained from another source. The intron may be homologous or heterologous to the transgene and/or to the transgenic mammal. The position of the intron with respect to the promoter and the transgene is important, as the intron must be transcribed to be effective. As such, where the transgene is a cDNA sequence, the preferred position for the intron is 3′ to the transcription start site, and 5′ to the polyA transcription termination sequence. Preferably for cDNA transgenes, the intron will be located on one side or the other (i.e., 5′ or 3′) of the transgene sequence such that it does not interrupt the transgene sequence. Any intron from any source, including any viral, prokaryotic and eukaryotic (plant or animal) organisms, may be used to express VEGF-B or PDGF, provided that it is compatible with the host cell(s) into which it is inserted. Also included herein are synthetic introns. Optionally, more than one intron may be used in the vector.

Preferred vectors for recombinant expression of VEGF-B or PDGF protein are those that are compatible with bacterial, insect, and mammalian host cells. Such vectors include, inter alia, pCRII (Invitrogen Company, San Diego, Calif.), pBSII (Stratagene Company, La Jolla, Calif.), and pETL (BlueBacII; Invitrogen).

After the vector has been constructed and a VEGF-B or PDGF nucleic acid has been inserted into the proper site of the vector, the completed vector may be inserted into a suitable host cell for amplification and/or VEGF-B or PDGF polypeptide expression. The host cells typically used include, without limitation: Prokaryotic cells such as gram negative or gram positive cells, i.e., any strain of E. coli, Bacillus, Streptomyces, Saccharomyces, Salmonella, and the like; eukaryotic cells such as CHO (Chinese hamster ovary) cells, human kidney 293 cells, COS-7 cells; insect cells such as Sf4, Sf5, Sf9, and Sf21 and High 5 (all from the Invitrogen Company, San Diego, Calif.); and various yeast cells such as Saccharomyces and Pichia.

Insertion (also referred to as “transformation” or “transfection”) of the vector into the selected host cell may be accomplished using such methods as calcium chloride, electroporation, microinjection, lipofection or the DEAE-dextran method. The method selected will in part be a function of the type of host cell to be used. These methods and other suitable methods are well known to the skilled artisan, and are set forth, for example, in Sambrook, et al., supra.

The host cells containing the vector (i.e., transformed or transfected) may be cultured using standard media well known to the skilled artisan. The media will usually contain all nutrients necessary for the growth and survival of the cells. Suitable media for culturing E. coli cells are for example, Luria Broth (LB) and/or Terrific Broth (TB). Suitable media for culturing eukaryotic cells are RPMI 1640, MEM, DMEM, all of which may be supplemented with serum and/or growth factors as required by the particular cell line being cultured. A suitable medium for insect cultures is Grace's medium supplemented with yeastolate, lactalbumin hydrolysate, and/or fetal calf serum as necessary.

Typically, an antibiotic or other compound useful for selective growth of the transformed cells only is added as a supplement to the media. The compound to be used will be dictated by the selectable marker element present on the plasmid with which the host cell was transformed. For example, where the selectable marker element is kanamycin resistance, the compound added to the culture medium will be kanamycin.

The amount of VEGF-B or PDGF polypeptide produced in the host cell can be evaluated using standard methods known in the art. Such methods include, without limitation, Western blot analysis, SDS-polyacrylamide gel electrophoresis, non-denaturing gel electrophoresis, HPLC separation, immunoprecipitation, and/or activity assays such as VEGFR-1, PDGFR-α, or PDGFR-β binding assays or cell stimulation assays.

Purification of VEGF-B or PDGF Polypeptides

VEGF-B polypeptides are preferably expressed and purified as described in U.S. Pat. No. 6,331,301, incorporated herein by reference.

If the VEGF-B or PDGF polypeptide has been designed to be secreted from the host cells, the majority of polypeptide will likely be found in the cell culture medium. If, however, the VEGF-B or PDGF polypeptide is not secreted from the host cells, it will be present in the cytoplasm (for eukaryotic, gram positive bacteria, and insect host cells) or in the periplasm (for gram negative bacteria host cells).

For intracellular VEGF-B or PDGF, the host cells are first disrupted mechanically or osmotically to release the cytoplasmic contents into a buffered solution. The polypeptide is then isolated from this solution.

Purification of VEGF-B or PDGF polypeptide from solution can be accomplished using a variety of techniques. If the polypeptide has been synthesized such that it contains a tag such as Hexahistidine (VEGF-B/hexaHis or PDGF/hexaHis) or other small peptide at either its carboxyl or amino terminus, it may essentially be purified in a one-step process by passing the solution through an affinity column where the column matrix has a high affinity for the tag or for the polypeptide directly (i.e., a monoclonal antibody specifically recognizing VEGF-B or PDGF). For example, polyhistidine binds with great affinity and specificity to nickel, thus an affinity column of nickel (such as the Qiagen nickel columns) can be used for purification of VEGF-B/polyHis or PDGF/polyHis. (See, for example, Ausubel, et al., eds., “Current Protocols In Molecular Biology,” Section 10.11.8, John Wiley & Sons, New York (1993)).

The strong affinity of VEGF-B for its receptor VEGFR-1 permits affinity purification of VEGF-B using an affinity matrix comprising VEGFR-1 extracellular domain. The strong affinity of PDGF-A for the PDGF receptor-α, the strong affinity for the PDGF-B for the PDGF receptors, the strong affinity of PDGF-C for the PDGF receptor-α and the strong affinity of PDGF-D receptors permit the affinity purification of these PDGFs using PDGF receptor-α and B extracellular domain. In addition, where the VEGF-B or PDGF polypeptide has no tag and no antibodies are available, other well known procedures for purification can be used. Such procedures include, without limitation, ion exchange chromatography, molecular sieve chromatography, HPLC, native gel electrophoresis in combination with gel elution, and preparative isoelectric focusing (“Isoprime” machine/technique, Hoefer Scientific). In some cases, two or more of these techniques may be combined to achieve increased purity. Preferred methods for purification include polyHistidine tagging and ion exchange chromatography in combination with preparative isoelectric focusing.

VEGF-B or PDGF polypeptide found in the periplasmic space of the bacteria or the cytoplasm of eukaryotic cells, the contents of the periplasm or cytoplasm, including inclusion bodies (bacteria) if the processed polypeptide has formed such complexes, can be extracted from the host cell using any standard technique known to the skilled artisan. For example, the host cells can be lysed to release the contents of the periplasm by French press, homogenization, and/or sonication. The homogenate can then be centrifuged.

If the VEGF-B or PDGF polypeptide has formed inclusion bodies in the periplasm, the inclusion bodies can often bind to the inner and/or outer cellular membranes and thus will be found primarily in the pellet material after centrifugation. The pellet material can then be treated with a chaotropic agent such as guanidine or urea to release, break apart, and solubilize the inclusion bodies. The VEGF-B or PDGF polypeptide in its now soluble form can then be analyzed using gel electrophoresis, immunoprecipitation or the like. If it is desired to isolate the VEGF-B or PDGF polypeptide, isolation may be accomplished using standard methods such as those set forth below and in Marston, et al., Meth. Enz., 182:264-275 (1990).

If VEGF-B or PDGF polypeptide inclusion bodies are not formed to a significant degree in the periplasm of the host cell, the VEGF-B or PDGF polypeptide will be found primarily in the supernatant after centrifugation of the cell homogenate, and the VEGF-B or PDGF polypeptide can be isolated from the supernatant using methods such as those set forth below.

In those situations where it is preferable to partially or completely isolate the VEGF-B or PDGF polypeptide, purification can be accomplished using standard methods well known to the skilled artisan. Such methods include, without limitation, separation by electrophoresis followed by electroelution, various types of chromatography (immunoaffinity, molecular sieve, and/or ion exchange), and/or high pressure liquid chromatography. In some cases, it may be preferable to use more than one of these methods for complete purification.

Anti-VEGF-B or Anti-PDGF Therapeutic Compounds

Anti-VEGF-B or Anit-PDGF therapies as discussed below include, but are not limited to antibody, aptamer, antisense and interference RNA techniques and therapies. These therapies are directed to myelosuppression instead of myelopoiesis. Whereas myelosuppression is often what one seeks to treat, for some conditions and disease states, such as in leukemia and lymphoma, myelosuppression may be desirable.

Therapeutic Anti-VEGF-B or Anti-PDGF Antibodies

Anti-VEGF-B antibodies as described in U.S. Pat. No. 6,331,301 are also contemplated for use in practicing the present invention. Such antibodies can be used for VEGF-B purification as described above, or therapeutically where inhibition of VEGF-B is desired (e.g., to achieve myelosuppressive effects).

Polyclonal or monoclonal therapeutic anti-VEGF-B or Anti-PDGF antibodies useful in practicing this invention may be prepared in laboratory animals or by recombinant DNA techniques using the following methods. Polyclonal antibodies to the VEGF-B or PDGF molecule or a fragment thereof containing the target amino acid sequence generally are raised in animals by multiple subcutaneous (sc) or intraperitoneal (ip) injections of the VEGF-B or PDGF molecule in combination with an adjuvant such as Freund's adjuvant (complete or incomplete). To enhance immunogenicity, it may be useful to first conjugate the VEGF-B or PDGF molecule or a fragment containing the target amino acid sequence of to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl, or R.sup. 1 N═C=NR, where R and R¹ are different alkyl groups. Alternatively, VEGF-B-immunogenic conjugates can be produced recombinantly as fusion proteins.

Animals are immunized against the immunogenic VEGF-B or PDGF conjugates or derivatives (such as a fragment containing the target amino acid sequence) by combining about 1 mg or about 1 microgram of conjugate (for rabbits or mice, respectively) with about 3 volumes of Freund's complete adjuvant and injecting the solution intradermally at multiple sites. Approximately 7 to 14 days later, animals are bled and the serum is assayed for anti-VEGF-B or PDGF titer. Animals are boosted with antigen repeatedly until the titer plateaus. Preferably, the animal is boosted with the same VEGF-B or PDGF molecule or fragment thereof as was used for the initial immunization, but conjugated to a different protein and/or through a different cross-linking agent. In addition, aggregating agents such as alum are used in the injections to enhance the immune response.

Monoclonal antibodies may be prepared by recovering spleen cells from immunized animals and immortalizing the cells in conventional fashion, e.g. by fusion with myeloma cells. The clones are then screened for those expressing the desired antibody. The monoclonal antibody preferably does not cross-react with other VEGF or PDGF family members.

Preparation of antibodies using recombinant DNA methods such as the phagemid display method, may be accomplished using commercially available kits, as for example, the Recombinant Phagemid Antibody System available from Pharmacia (Uppsala, Sweden), or the SurfZAP™ phage display system (Stratagene Inc., La Jolla, Calif.).

Preferably, antibodies for administration to humans, although prepared in a laboratory animal such as a mouse, will be “humanized”, or chimeric, i.e. made to be compatible with the human immune system such that a human patient will not develop an immune response to the antibody. Even more preferably, human antibodies which can now be prepared using methods such as those described for example, in Lonberg, et al., Nature Genetics, 7:13-21 (1994) are preferred for therapeutic administration to patients.

A. Humanization of Anti-VEGF-B or Anti-PDGF Monoclonal Antibodies

VEGF-B-neutralizing antibodies comprise one class of therapeutics useful as VEGF-B antagonists. PDGF-neutralizing antibodies comprise one class of therapeutics useful as PDGF antagonists. Following are protocols to improve the utility of anti-VEGF-B monoclonal antibodies as therapeutics in humans, by “humanizing” the monoclonal antibodies to improve their serum half-life and render them less immunogenic in human hosts (i.e., to prevent human antibody response to non-human anti-VEGF-B or non-human anti-PDGF antibodies).

The principles of humanization have been described in the literature and are facilitated by the modular arrangement of antibody proteins. To minimize the possibility of binding complement, a humanized antibody of the IgG4 isotype is preferred.

For example, a level of humanization is achieved by generating chimeric antibodies comprising the variable domains of non-human antibody proteins of interest, such as the anti-VEGF-B monoclonal antibodies described herein, with the constant domains of human antibody molecules. (See, e.g., Morrison and Oi, Adv. Immunol., 44:65-92 (1989)). The variable domains of VEGF-B neutralizing anti-VEGF-B antibodies are cloned from the genomic DNA of a B-cell hybridoma or from cDNA generated from mRNA isolated from the hybridoma of interest. The V region gene fragments are linked to exons encoding human antibody constant domains, and the resultant construct is expressed in suitable mammalian host cells (e.g., myeloma or CHO cells).

To achieve an even greater level of humanization, only those portions of the variable region gene fragments that encode antigen-binding complementarity determining regions (“CDR”) of the non-human monoclonal antibody genes are cloned into human antibody sequences. (See, e.g., Jones, et al., Nature, 321:522-525 (1986); Riechmann, et al., Nature, 332:323-327 (1988); Verhoeyen, et al., Science, 239:1534-36 (1988); and Tempest, et al., Bio/Technology, 9:266-71 (1991)). If necessary, the beta-sheet framework of the human antibody surrounding the CDR3 regions also is modified to more closely mirror the three dimensional structure of the antigen-binding domain of the original monoclonal antibody. (See, Kettleborough, et al., Protein Engin., 4:773-783 (1991); and Foote, et al., J. Mol. Biol., 224:487-499 (1992)).

In an alternative approach, the surface of a non-human monoclonal antibody of interest is humanized by altering selected surface residues of the non-human antibody, e.g., by site-directed mutagenesis, while retaining all of the interior and contacting residues of the non-human antibody. See Padlan, Molecular Immunol., 28(4/5):489-98 (1991).

The foregoing approaches are employed using VEGF-B-neutralizing anti-VEGF-B monoclonal antibodies and the hybridomas that produce them to generate humanized VEGF-B-neutralizing antibodies useful as therapeutics to treat or palliate conditions wherein VEGF-B expression is detrimental.

The foregoing approaches are employed using PDGF-neutralizing anti-PDGF monoclonal antibodies and the hybridomas that produce them to generate humanized PDGF-neutralizing antibodies useful as therapeutics to treat or palliate conditions wherein PDGF expression is detrimental.

B. Human VEGF-B-Neutralizing or Human PDGF-Neutralizing Antibodies from Phage Display

Human VEGF-B-neutralizing or PDGF-neutralizing antibodies are generated by phage display techniques such as those described in Aujame, et al., Human Antibodies, 8(4):155-168 (1997); Hoogenboom, TIBTECH, 15:62-70 (1997); and Rader, et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is panned (screened) for VEGF-B-specific or PDGF-specific phage-antibodies using labeled or immobilized VEGF-B or PDGF respectively as antigen-probe.

C. Human VEGF-B-Neutralizing or Human PDGF-Neutralizing Antibodies from Transgenic Mice

Human VEGF-B-neutralizing antibodies are generated in transgenic mice essentially as described in Bruggemann and Neuberger, Immunol. Today, 17(8):391-97 (1996) and Bruggemann and Taussig, Curr. Opin. Biotechnol., 8:455-58 (1997). Transgenic mice carrying human V-gene segments in germline configuration and that express these transgenes in their lymphoid tissue are immunized with VEGF-B or PDGF composition using conventional immunization protocols. Hybridomas are generated using B cells from the immunized mice using conventional protocols and screened to identify hybridomas secreting anti-VEGF-B or anti-PDGF human antibodies (e.g., as described above).

D. Bispecific Antibodies

Bispecific antibodies that specifically bind to one protein (e.g., VEGF-B or PDGF) and that specifically bind to other antigens relevant to pathology and/or treatment are produced, isolated, and tested using standard procedures that have been described in the literature. See, e.g., Pluckthun & Pack, Immunotechnology, 3:83-105 (1997); Carter, et al., J. Hematotherapy, 4: 463-470 (1995); Renner & Pfreundschuh, Immunological Reviews, 1995, No. 145, pp. 179-209; Pfreundschuh U.S. Pat. No. 5,643,759; Segal, et al., J. Hematotherapy, 4: 377-382 (1995); Segal, et al., Immunobiology, 185: 390-402 (1992); and Bolhuis, et al., Cancer Immunol. Immunother., 34: 1-8 (1991), all of which are incorporated herein by reference in their entireties.

Anti-VEGF-B and Anti-PDGF Aptamers

Recent advances in the field of combinatorial sciences have identified short polymer sequences with high affinity and specificity to a given target. For example, SELEX technology has been used to identify DNA and RNA aptamers with binding properties that rival mammalian antibodies, the field of immunology has generated and isolated antibodies or antibody fragments which bind to a myriad of compounds and phage display has been utilized to discover new peptide sequences with very favorable binding properties. Based on the success of these molecular evolution techniques, it is certain that ligands can be created which bind to any molecule. Curiously, in each case, a loop structure is often involved with providing the desired binding attributes as in the case of: aptamers which often utilize hairpin loops created from short regions without complimentary base pairing, naturally derived antibodies that utilize combinatorial arrangement of looped hyper-variable regions and new phage display libraries utilizing cyclic peptides that have shown improved results when compare to linear peptide phage display results. Thus, sufficient evidence has been generated to suggest that high affinity ligands can be created and identified by combinatorial molecular evolution techniques. For the present invention, molecular evolution techniques can be used to isolate ligands specific for VEGF-B, to be used in a manner analogous to that discussed above for anti-VEGF-B antibodies. For more on aptamers, see generally, Gold, L., Singer, B., He, Y. Y., Brody. E., “Aptamers As Therapeutic And Diagnostic Agents,” J. Biotechnol. 74:5-13 (2000).

Anti-sense Molecules and Therapy

Another class of VEGF-B or PDGF inhibitors useful in the present invention is isolated antisense nucleic acid molecules that can hybridize to, or are complementary to, the nucleic acid molecule comprising the VEGF-B or PDGF nucleotide sequence, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire VEGF-B or PDGF coding strand, or to only a portion thereof. Nucleic acid molecules encoding fragments, homologs, derivatives and analogs of VEGF-B or PDGF or antisense nucleic acids complementary to a VEGF-B or PDGF nucleic acid sequence are additionally provided.

In one embodiment, an antisense nucleic acid molecule is antisense to a “coding region” of the coding strand of a nucleotide sequence encoding a VEGF-B or PDGF protein. The term “coding region” refers to the region of the nucleotide sequence comprising codons that are translated into amino acid residues. In another embodiment, the antisense nucleic acid molecule is antisense to a “conceding region” of the coding strand of a nucleotide sequence encoding the VEGF-B or PDGF protein. The term “conceding region” refers to 5′ and 3′ sequences that flank the coding region and that are not translated into amino acids (i.e., also referred to as 5′ and 3′ untranslated regions).

Given the coding strand sequences encoding the VEGF-B or PDGF protein disclosed herein, antisense nucleic acids of the invention can be designed according to the rules of Watson and Crick or Hoogsteen base pairing. The antisense nucleic acid molecule can be complementary to the entire coding region of VEGF-B or PDGF mRNA, but more preferably is an oligonucleotide that is antisense to only a portion of the coding or noncoding region of VEGF-B or PDGF mRNA. For example, the antisense oligonucleotide can be complementary to the region surrounding the translation start site of VEGF-B mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following section).

The antisense nucleic acid molecules of the invention are typically administered to a subject or generated in situ such that they hybridize with or bind to cellular mRNA and/or genomic DNA encoding VEGF-B or PDGF to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., Nucl. Acids Res., 15:6625-6641 (1987). The antisense nucleic acid molecule can also comprise a 2′-o-methylribonucleotide (see, e.g., Inoue, et al. Nucl. Acids Res., 15:6131-6148 (1987)) or a chimeric RNA-DNA analogue (see, e.g., Inoue, et al., FEBS Lett., 215:327-330 (1987)).

Production and delivery of antisense molecules are facilitated by providing a vector comprising an anti-sense nucleotide sequence complementary to at least a part of the VEGF-B or PDGF DNA sequence. According to a yet further aspect of the invention such a vector comprising an anti-sense sequence may be used to inhibit, or at least mitigate, VEGF-B or PDGF expression. The use of a vector of this type to inhibit VEGF-B or PDGF expression is favored in instances where VEGF-B or PDGF expression is associated with a particular disease state.

Anti-VEGF-B or Anit-PDGF RNA Interference

Use of RNA Interference to inactivate or modulate VEGF-B or PDGF expression is also contemplated by this invention. RNA interference is described in U.S. Patent Appl. No. 2002-0162126, and Hannon, G., J. Nature, 11:418:244-51 (2002). “RNA interference,” “post-transcriptional gene silencing,” “quelling”—these terms have all been used to describe similar effects that result from the overexpression or misexpression of transgenes, or from the deliberate introduction of double-stranded RNA into cells (reviewed in Fire, A., Trends Genet 15:358-363 (1999); Sharp, P. A., Genes Dev., 13:139-141 (1999); Hunter, C., Curr. Biol., 9:R440-R442 (1999); Baulcombe, D. C., Curr. Biol. 9:R599-R601 (1999); Vaucheret, et al. Plant J. 16:651-659 (1998), all incorporated by reference. RNA interference, commonly referred to as RNAi, offers a way of specifically and potently inactivating a cloned gene.

Therapeutic Compositions and Administration

Therapeutic formulations of the compositions useful for practicing the present invention such as VEGF-B polypeptides, polynucleotides, or antibodies may be prepared for storage by mixing the selected composition having the desired degree of purity with optional physiologically pharmaceutically-acceptable carriers, excipients, or stabilizers (Remington's Pharmaceutical Sciences, 18th edition, A. R. Gennaro, ed., Mack Publishing Company (1990)) in the form of a lyophilized cake or an aqueous solution. Acceptable carriers, excipients or stabilizers are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

The composition to be used for in vivo administration should be sterile. This is readily accomplished by filtration through sterile filtration membranes, prior to or following lyophilization and reconstitution. The composition for parenteral administration ordinarily will be stored in lyophilized form or in solution.

Therapeutic compositions generally are placed into a container having a sterile access port, for example, an intravenous solution bag or vial having a stopper pierceable by a hypodermic injection needle. The route of administration of the composition is in accord with known methods, e.g. oral, injection or infusion by intravenous, intraperitoneal, intracerebral, intramuscular, intraocular, intraarterial, or intralesional routes, or by sustained release systems or implantation device. Where desired, the compositions may be administered continuously by infusion, bolus injection or by implantation device.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman, et al., Biopolymers, 22: 547-556 (1983)), poly (2-hydroxyethyl-methacrylate) (Langer, et al., J. Biomed. Mater. Res., 15:167-277 (1981) and Langer, Chem. Tech., 12:98-105 (1982)), ethylene vinyl acetate (Langer, et al., supra) or poly-D(−)-3-hydroxybutyric acid (EP 133,988). Sustained-release compositions also may include liposomes, which can be prepared by any of several methods known in the art (e.g., DE 3,218,121; Epstein, et al., Proc. Natl. Acad. Sci. USA, 82:3688-3692 (1985); Hwang, et al., Proc. Natl. Acad. Sci. USA, 77:4030-4034 (1980); EP 52,322; EP 36,676; EP 88,046; EP 143,949).

An effective amount of the compositions to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. A typical daily dosage may range from about 1 μg/kg to up to 100 mg/kg or more, depending on the factors mentioned above. Typically, a clinician will administer the composition until a dosage is reached that achieves the desired effect. The progress of this therapy is easily monitored by conventional assays designed to evaluate myelosuppression or the particular conditions of interest in a particular subject.

Pharmaceutical compositions may be produced by admixing a pharmaceutically effective amount of VEGF-B protein with one or more suitable carriers or adjuvants such as water, mineral oil, polyethylene glycol, starch, talcum, lactose, thickeners, stabilizers, suspending agents, etc. Such compositions may be in the form of solutions, suspensions, tablets, capsules, creams, salves, ointments, or other conventional forms.

VEGF-B or PDGFs can be used directly to practice materials and methods of the invention, but in preferred embodiments, the compounds are formulated with pharmaceutically acceptable diluents, adjuvants, excipients, or carriers. The phrase “pharmaceutically or pharmacologically acceptable” refers to molecular entities and compositions that do not produce adverse, allergic, or other untoward reactions when administered to an animal or a human, e.g., orally, topically, transdermally, parenterally, by inhalation spray, vaginally, rectally, or by intracranial injection. (The term parenteral as used herein includes subcutaneous injections, intravenous, intramuscular, intracistemal injection, or infusion techniques. Administration by intravenous, intradermal, intramusclar, intramammary, intraperitoneal, intrathecal, retrobulbar, intrapulmonary injection and/or surgical implantation at a particular site is contemplated as well.) Generally, this will also entail preparing compositions that are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Polynucleotide-Based VEGF-B or PDGF Therapies

In one embodiment, the therapeutic effects of VEGF-B or PDGFs on stem cell recruitment, proliferation, and/or differentiation are achieved by administration of VEGF-B or PDGF encoding polynucleotides (including vectors comprising such polynucleotides) to a subject that will benefit from the VEGF-B or PDGF.

For these embodiments, an exemplary expression construct comprises a virus or engineered construct derived from a viral genome. The expression construct generally comprises a nucleic acid encoding the gene to be expressed and also additional regulatory regions that will effect the expression of the gene in the cell to which it is administered. Such regulatory regions include for example promoters, enhancers, polyadenylation signals and the like.

It is now widely recognized that DNA may be introduced into a cell using a variety of viral vectors. In such embodiments, expression constructs comprising viral vectors containing the genes of interest may be adenoviral (see, for example, U.S. Pat. No. 5,824,544; U.S. Pat. No. 5,707,618; U.S. Pat. No. 5,693,509; U.S. Pat. No. 5,670,488; U.S. Pat. No. 5,585,362; each incorporated herein by reference), retroviral (see, for example, U.S. Pat. No. 5,888,502; U.S. Pat. No. 5,830,725; U.S. Pat. No. 5,770,414; U.S. Pat. No. 5,686,278; U.S. Pat. No. 4,861,719 each incorporated herein by reference), adeno-associated viral (see, for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479 each incorporated herein by reference), an adenoviral-adenoassociated viral hybrid (see, for example, U.S. Pat. No. 5,856,152 incorporated herein by reference) or a vaccinia viral or a herpesviral (see, for example, U.S. Pat. No. 5,879,934; U.S. Pat. No. 5,849,571; U.S. Pat. No. 5,830,727; U.S. Pat. No. 5,661,033; U.S. Pat. No. 5,328,688 each incorporated herein by reference) vector.

In other embodiments, non-viral delivery is contemplated. These include calcium phosphate precipitation (Graham and Van Der Eb, Virology, 52:456-467 (1973); Chen and Okayama, Mol. Cell Biol., 7:2745-2752, (1987); Rippe, et al., Mol. Cell Biol., 10:689-695 (1990)), DEAE-dextran (Gopal, Mol. Cell Biol., 5:1188-1190 (1985)), electroporation (Tur-Kaspa, et al., Mol. Cell Biol., 6:716-718, (1986); Potter, et al., Proc. Nat. Acad. Sci. USA, 81:7161-7165, (1984)), direct microinjection (Harland and Weintraub, J. Cell Biol., 101: 1094-1099 (1985)), DNA-loaded liposomes (Nicolau and Sene, Biochim. Biophys. Acta, 721:185-190 (1982); Fraley, et al., Proc. Natl. Acad. Sci. USA, 76:3348-3352 (1979); Felgner, Sci. Am., 276(6):102-6 (1997); Felgner, Hum. Gene Ther., 7(15):1791-3, (1996)), cell sonication (Fechheimer, et al., Proc. Natl. Acad. Sci. USA, 84:8463-8467 (1987)), gene bombardment using high velocity microprojectiles (Yang, et al., Proc. Natl. Acad. Sci. USA, 87:9568-9572 (1990)), and receptor-mediated transfection (Wu and Wu, J. Biol. Chem., 262:4429-4432 (1987); Wu and Wu, Biochemistry, 27:887-892 (1988); Wu and Wu, Adv. Drug Delivery Rev., 12:159-167 (1993)).

In a particular embodiment of the invention, the expression construct (or indeed the peptides discussed above) may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, “In Liver Diseases, Targeted Diagnosis And Therapy Using Specific Receptors And Ligands,” Wu, G., Wu, C., ed., New York: Marcel Dekker, pp. 87-104 (1991)). The addition of DNA to cationic liposomes causes a topological transition from liposomes to optically birefringent liquid-crystalline condensed globules (Radler, et al., Science, 275(5301):810-4, (1997)). These DNA-lipid complexes are potential non-viral vectors for use in gene therapy and delivery.

Liposome-mediated nucleic acid delivery and expression of foreign DNA in vitro has been very successful. Also contemplated in the present invention are various commercial approaches involving “lipofection” technology. In certain embodiments of the invention, the liposome may be complexed with a hemagglutinating virus (HVJ). This has been shown to facilitate fusion with the cell membrane and promote cell entry of liposome-encapsulated DNA (Kaneda, et al., Science, 243:375-378 (1989)). In other embodiments, the liposome may be complexed or employed in conjunction with nuclear nonhistone chromosomal proteins (HMG-1) (Kato, et al., J. Biol. Chem., 266:3361-3364 (1991)). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems that can be employed to deliver a nucleic acid encoding a therapeutic gene into cells include receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993), supra).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu (1987), supra) and transferrin (Wagner, et al., Proc. Nat'l. Acad. Sci. USA, 87(9):3410-3414 (1990)). Recently, a synthetic neoglycoprotein, which recognizes the same receptor as ASOR, has been used as a gene delivery vehicle (Ferkol, et al., FASEB. J., 7:1081-1091 (1993); Perales, et al., Proc. Natl. Acad. Sci., USA 91:4086-4090 (1994)) and epidermal growth factor (EGF) has also been used to deliver genes to squamous carcinoma cells (Myers, EPO 0273085).

In other embodiments, the delivery vehicle may comprise a ligand and a liposome. For example, Nicolau, et al., Methods Enzymol., 149:157-176 (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a particular cell type by any number of receptor-ligand systems with or without liposomes.

In another embodiment of the invention, the expression construct may simply consist of naked recombinant DNA or plasmids. Transfer of the construct may be performed by any of the methods mentioned above that physically or chemically permeabilize the cell membrane. This is applicable particularly for transfer in vitro, however, it may be applied for in vivo use as well. Dubensky, et al., Proc. Nat. Acad. Sci. USA, 81:7529-7533 (1984) successfully injected polyomavirus DNA in the form of CaPO₄ precipitates into liver and spleen of adult and newborn mice demonstrating active viral replication and acute infection. Benvenisty and Neshif, Proc. Nat. Acad. Sci. USA, 83:9551-9555 (1986) also demonstrated that direct intraperitoneal injection of CaPO₄ precipitated plasmids results in expression of the transfected genes.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein, et al., Nature, 327:70-73 (1987)). Several devices for accelerating small particles have been developed. One such device relies on a high voltage discharge to generate an electrical current, which in turn provides the motive force (Yang, et al., Proc. Natl. Acad. Sci USA, 87:9568-9572 (1990)). The microprojectiles used have consisted of biologically inert substances such as tungsten or gold beads.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10×⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various cell types. For practically any cell, tissue or organ type, systemic delivery is contemplated. In other embodiments, a variety of direct, local and regional approaches may be taken. For example, the cell, tissue or organ may be directly injected with the expression vector or protein.

Preferred promoters for gene therapy for use in this invention include cytomegalovirus (CMV) promoter/enhancer, long terminal repeat (LTR) of retroviruses, keratin 14 promoter, and a myosin heavy chain promoter.

In a different embodiment, ex vivo gene therapy is contemplated. In an ex vivo embodiment, cells from the patient are removed and maintained outside the body for at least some period of time. During this period, a therapy is delivered, after which the cells are reintroduced into the patient; preferably, any tumor cells in the sample have been killed.

The techniques, procedures and methods outlined herein for VEGF-B and PDGF are applicable to any and all of the growth factors of the present invention.

The invention may be more readily understood by reference to the following examples, are given to illustrate the invention and not in any way to limit its scope.

### EXAMPLE 1

### Effects of VEGF-B or VEGF-C Gene Therapy on White Blood Cell Counts

The following procedures were performed to elucidate the roles of certain growth factors and their receptors, including VEGF-B and its receptor VEGFR-1, on hematopoietic progenitor cells.

NMRI nu/nu mice (nude mice) received intravenous injection of adenoviruses encoding one of the following proteins: beta-galactosidase (1×10⁹ pfu), VEGF-C (3×10⁸ pfu), VEGF-C 156S (a mutant form of VEGF-C; 3×10⁸ pfu, see U.S. Pat. Number 6,361,946), a soluble form of the VEGFR-3 extracellular domain (VEGFR-3-Ig fusion protein; 1×10⁹ pfu), or VEGF-B (50:50 mixture of VEGF-B₁₆₇ and VEGF-B₁₈₆) (1×10⁹ pfu). The beta-galactosidase served as a negative control.

Four days after the injection the mice were sacrificed, blood was collected and white blood cell (WBC) counts from the peripheral blood were measured using flow cytometry. In addition, WBCs of the blood were treated with anti-VEGFR-3 antibodies and stained with phycoerythrin-conjugated rat anti-mouse IgG2A for fluorescence activated cell sorting (FACS) analysis. Red blood cells were lysed by a buffered ammonium chloride/potassium (ACK) lysing solution and 1×10⁵ white blood cells were used per sample.

The white blood cells were washed with PBS containing 2% fetal calf serum and incubated with Fc Block (BD Pharmingen) for 5 minutes followed by incubation with conjugated antibody for 30 minutes on ice. As a negative isotype control, phycoerythrin-conjugated rat IgG2A (BD Pharmingen) was used at the same concentration to measure the background signal. Cells were washed and analyzed in the LSR cytometer (Becton Dickinson). Other PE- or FITC-labeled antibodies used in the flow cytometry were anti-VEGFR-1, anti-Tie-2, anti-VEGFR-2, anti-CD34, anti-CD117, anti-CD11b, and anti-Ly-6G/C.

In mice treated with Ad-VEGF-C156S, a clear mobilization of hematopoietic cells expressing VEGFR-1, VEGFR-2 and VEGFR-3 to the peripheral blood was seen. Furthermore, the percentage of CD34+cells in the blood circulation was higher when compared to the Ad-LacZ treated mice.

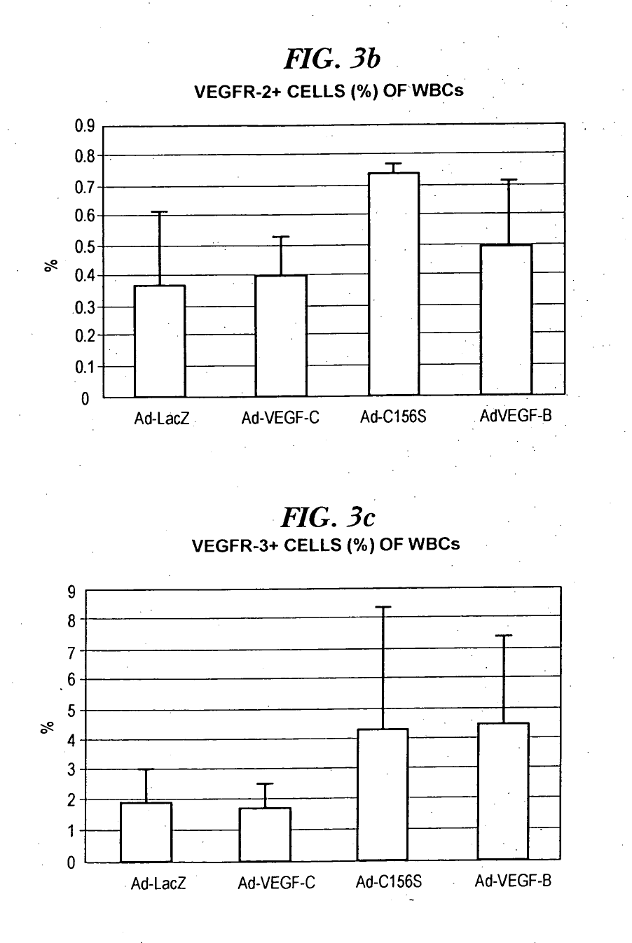

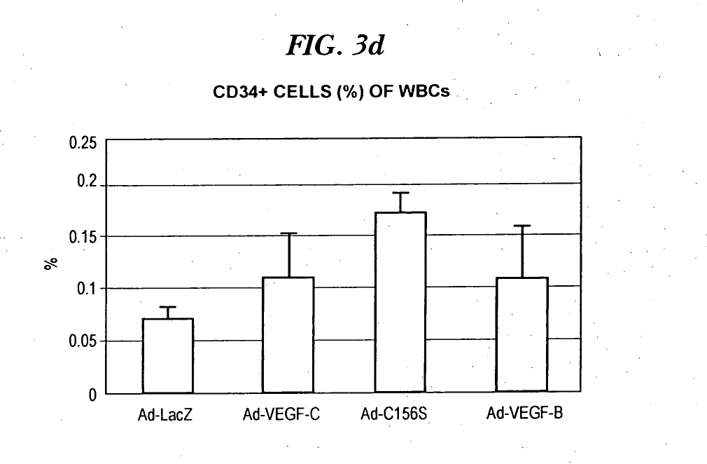

Elevated numbers of VEGFR-1⁺ cells were also present in the Ad-VEGF-C and Ad-VEGF-B groups (FIG. 3a-d). The results indicate that VEGF-C, and especially its VEGFR-3 specific mutant form VEGF-C156S, as well as VEGF-B can mobilize endothelial/hematopoietic progenitor cells from the bone marrow.

### EXAMPLE 2

### Recovery from Chemotherapy-Induced Myelosuppression

To study hematopoiesis during bone marrow recovery, FVB or NMRI wild-type mice (6-10 weeks old) were treated with a single i.v. injection of cytotoxic 5-fluorouracil (5-FU, 300 mg/kg, Pharmacia), which transiently depletes most of the circulating hematopoietic cells. Recovery with and without various exogenous growth factor treatments was studied. WBCs were analyzed as described in Example 1.

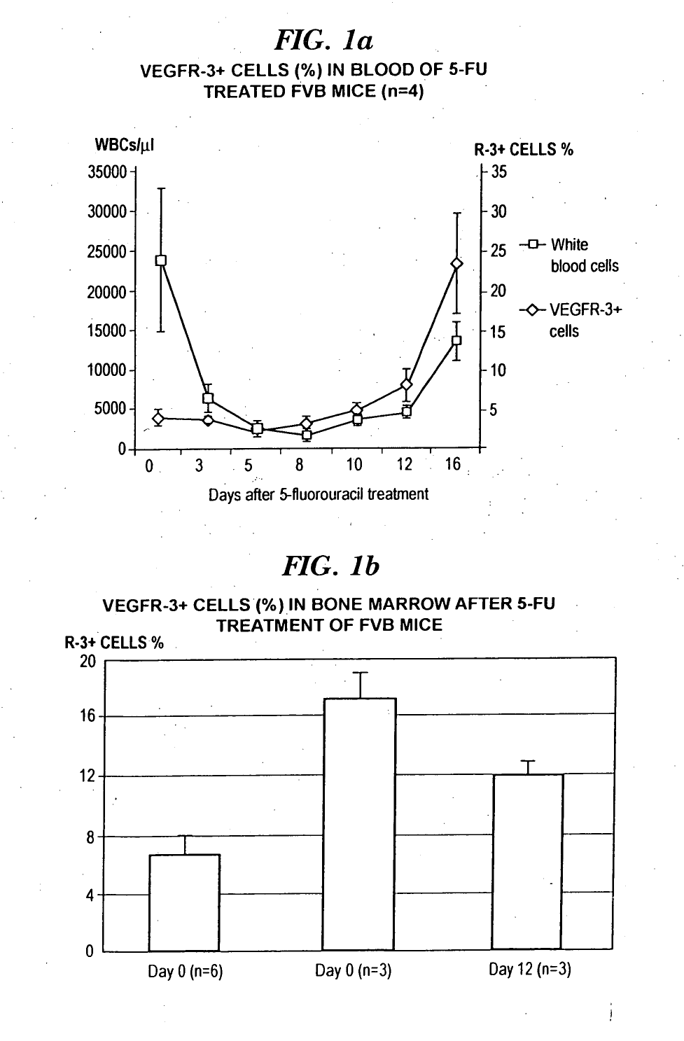

In a first experiment, the effects of 5-FU alone were studied. Before the myelosuppressive treatment, the peripheral blood contained about 4.1% white blood cells (WBCs) positive for VEGFR-3 staining. The percentage of VEGFR-3+cells in the blood increased beginning on day 5 after 5-FU treatment, and on day 16 the number was 23.7% (FIG. 1a). Also, in the bone marrow of the femur the percentage of VEGFR-3 positive cells was clearly elevated after 5-FU treatment (FIG. 1b).

In a second set of experiments, mice treated with 5-FU simultaneously received an intravenous injection of an adenoviruses encoding one of the following proteins: beta-galactosidase (1×10⁹ pfu), VEGF-C (3×10⁸ pfu), VEGF-C156S (3×10⁸ pfu), or soluble VEGFR-3 extracellular domain (VEGFR-3-1 g fusion protein; 1×10⁹ pfu).

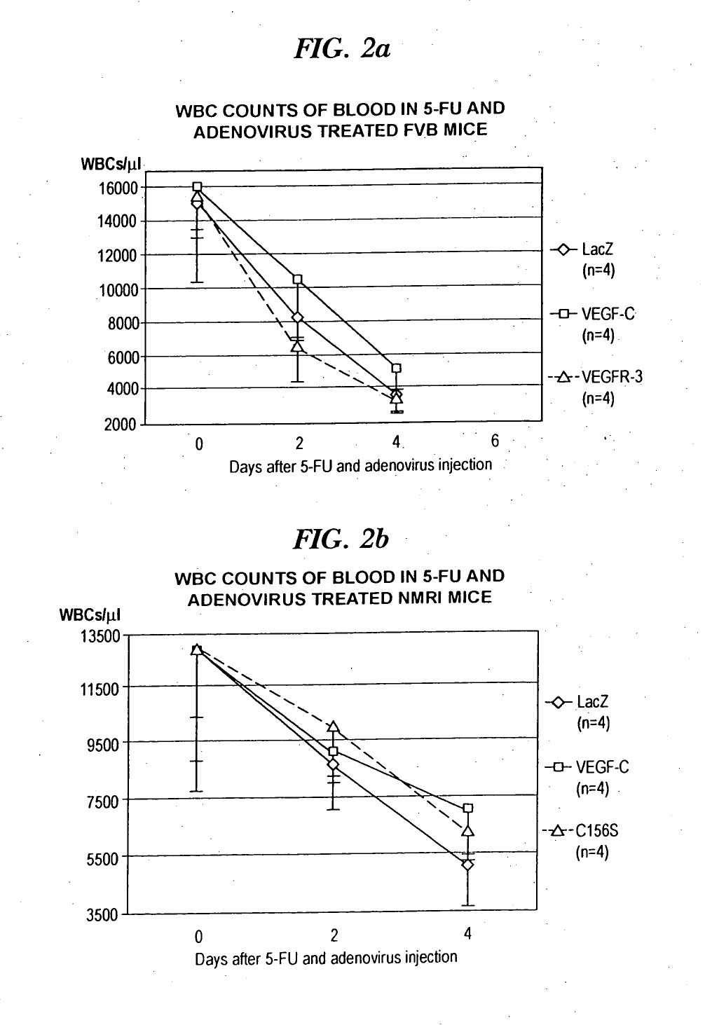

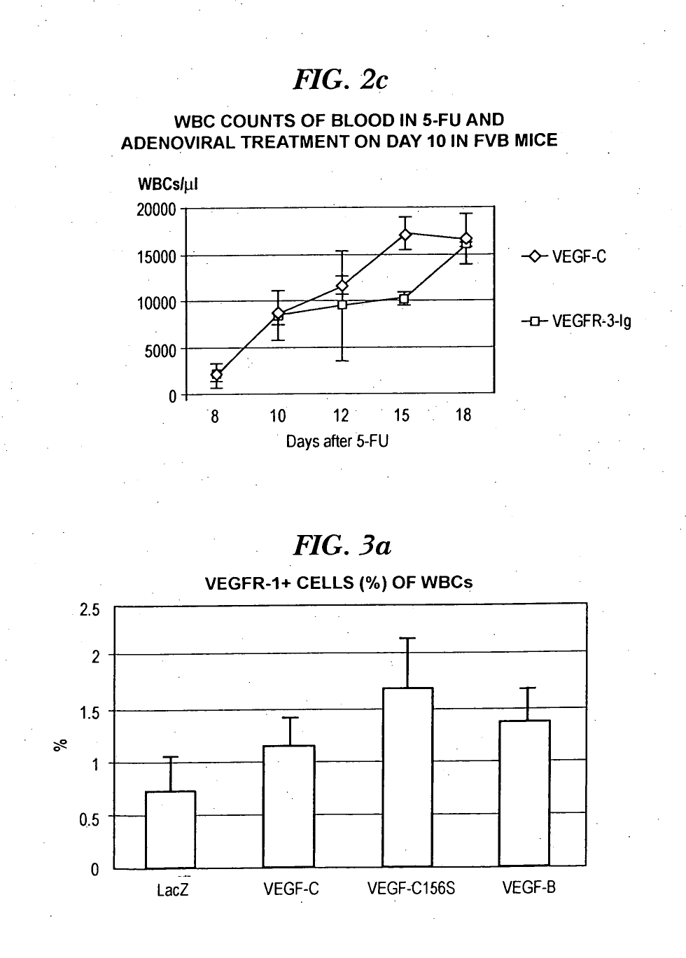

White blood counts (WBC) from the peripheral blood were measured after two or four days using the techniques described in Example 1. In Ad-VEGF-C treated mice, the number of WBC was higher during the first four days (+43% at day 4), whereas in AdVEGFR-3-Ig treated mice WBC count decreased more rapidly (−27% at day 2, −12% at day 4), when compared to Ad-LacZ treated mice (FIG. 2a). Furthermore, the injection of adenoviruses encoding VEGF-C156S, a mutant form of VEGF-C, which activates only VEGFR-3, also increased the WBC number in peripheral blood in the same 5-FU model (FIG. 2b).

The mice received second injections of adenoviruses encoding VEGF-C or VEGFR-3-Ig on day 10. In VEGFR-3-Ig treated mice, blocking the VEGFR-3 pathway inhibited the bone marrow recovery and elevation of the WBC number (FIG. 2c).

### EXAMPLE 3

### Effects of VEGF-B or PDGF Gene Therapy on White Blood Cell Counts

The following procedures are performed to elucidate the roles of certain growth factors and their receptors, including VEGF-B and its receptor VEGFR-1 and the PDGFs and their respective receptors, on hematopoietic progenitor cells.

NMRI nu/nu mice (nude mice), VEGF-B deficient mice (VEGF-B knock-out mice as described in Aase, et al., Circulation, 104:358-64 (2001) and Wanstall, et al., Card. Res., 55:361-368 (2002)), or PDGF (PDGF-A, PDGF-B, PDGF-C, or PDGF-D) deficient mice receive intravenous injection of adenoviruses encoding one or more of the following proteins at concentrations of 8×10⁷ to 6×10⁹ pfu: beta-galactosidase, VEGF-B₁₆₇, VEGF-B₁₈₆, a VEGF-B N-acetylated variant, PDGF-A, PDGF-B, PDGF-C, and PDGF-D. The beta-galactosidase serves as a negative control.

Four days after the viral injection, the mice are sacrificed, blood is collected and white blood cell (WBC) counts from the peripheral blood were measured after four days using flow cytometry. Red blood cells are lysed by a buffered ammonium chloride/potassium (ACK) lysing solution and 10⁵ white blood cells are used per sample. White blood cells are immunoanalyzed as in Example 1, and additionally with anti-PDGF-receptor-α and anti-PDGF-receptor-β antibodies.

The relative number of white blood cells expressing the various cell-surface markers indicative of stem cells are compared between control and experimental mice to evaluate the level of stem cell recruitment, differentiation and proliferation.

### EXAMPLE 4

### Myelosuppression and Recovery with VEGF-B and PDGFS

To study hematopoiesis during bone marrow recovery, FVB or NMRI wild-type mice (6-10 weeks old), VEGF-B-deficient or PDGF (PDGF-A, PDGF-B, PDGF-C, or PDGF-D) deficient mice are treated with a single i.v. injection of cytotoxic 5-fluorouracil (5-FU, 300 mg/kg, Pharmacia), which transiently depletes most of the circulating hematopoietic cells. Recovery with and without various exogenous growth factor treatments is studied. WBCs are analyzed as described in Example 1, and additionally with anti-PDGF-receptor-α and anti-PDGF-receptor-β antibodies.

In a first experiment, the effects of 5-FU alone are studied. Before the myelosuppressive treatment, the peripheral blood is analyzed according to Example 2, and additionally with anti-PDGF-receptor-α and anti-PDGF-receptor-β antibodies.

In a second set of experiments, mice are treated simultaneously with 5-FU and with adenoviruses (at concentrations of 8×10⁷ to 6×10⁹ pfu) containing transgenes encoding one or more of the following proteins: beta-galactosidase, VEGF-B₁₆₇, VEGF-B₁₈₆, a VEGF-B N-acetylated variant, PDGF-A, PDGF-B, PDGF-C, and PDGF-D. In addition, adenoviruses encoding solubilized PDGF receptor extracellular domain/IgG Fusion are tested.

White blood counts (WBC) from the peripheral blood are analyzed after two or four days using the techniques described in Example 2, and additionally with anti-PDGF-receptor-α and anti-PDGF-receptor-β antibodies.

The relative number of white blood cells expressing the various cell-surface markers indicative of stem cells (e.g., AC133, VEGFR-2 or -1, c-kit) are compared between control and experimental mice to evaluate the effects of each protein on stem cell recruitment, differentiation and proliferation.

### EXAMPLE 5

### Myelopoietic Protein Therapy

The following procedures are performed to elucidate the roles of certain growth factors and their receptors, including VEGF-B and its receptor VEGFR-1 and the PDGFs and their respective receptors, on hematopoietic progenitor cells.

NMRI nu/nu mice (nude mice), VEGF-B deficient mice (VEGF-B knock-out mice as described in Aase, et al., Circulation, 104:358-64 (2001) and Wanstall, et al., Card. Res., 55:361-368 (2002)), or PDGF (PDGF-A, PDGF-B, PDGF-C, or PDGF-D) deficient mice receive a control protein or one or more of the following growth factors: VEGF-B₁₆₇, VEGF-B₁₈₆, a VEGF-B N-acetylated variant, PDGF-A, PDGF-B, PDGF-C, and PDGF-D. Alternatively, the mice receive soluble receptor extracellular domain protein preparations (e.g., VEGFR-1-Ig, PDGFR-α-Ig, or PDGFR-β-Ig). Before administering the protein compositions, baseline white blood cells are characterized as described in Example 1. The mice receive an initial intravenous (IV) bolus dose of growth factor or control protein over 60 minutes. After a 48-hour observation period, the mice receive a 14-day course of continuous IV infusion of the growth factor or control protein. A variety of protein concentrations are tested. For example, the mice receive a total dose of either 0.5 1.0, 2.0, 4.0, or 8.0 μg/kg. On days 2, 6, 11, 16 and 24, white blood cells are characterized as described in Example 1.

The relative number of white blood cells expressing the various cell-surface markers indicative of stem cells are compared between control and experimental mice to evaluate the effects of each protein on stem cell recruitment, differentiation and proliferation.

### EXAMPLE 6

### Ex Vivo Expansion of Endothelial Progenitor Cells Derived from Subjects Treated with VEGF-B or PDGFS

The following experiments are performed to demonstrate the ability of VEGF-B and/or PDGF therapy to improve the efficacy and healing of a tissue, organ, or prosthetic graft or implant. This example is based on the methods of Kaushal, et al., “Functional Small-Diameter Neovessels Created Using Endothelial Progenitor Cells Expanded Ex Vivo,” Nat. Med., 7:1035-1040 (2001), which is incorporated herein in its entirety. A subject is treated with one or more of the following: VEGF-B₁₆₇, VEGF-B₁₈₆, a VEGF-B N-acetylated variant, PDGF-A, PDGF-B, PDGF-C, and PDGF-D or a control either in direct protein form or encoded by a polynucleotide as part of a gene therapy vector, such as a recombinant adenovirus, adeno-associated virus (AAV), plasmid or other vector, or naked DNA comprising a polynucleotide that encodes VEGF-B, a PDGF, or a fragment thereof. In one variation, VEGF-B and/or a PDGF protein is administered using implantable osmotic mini-pumps. The VEGF-B or PDGF therapy is performed to increase the quantity of circulating endothelial progenitor cells (EPCs).

After 2, 4, 6, 8, 12 or 14 days of treatment as described above, blood is drawn in heparinized tubes and the leukocytes are isolated on a Histopaque density gradient (Sigma) for 30 minutes at 1000 g using Accuspin tubes (Sigma). The leukocytes are resuspended in growth medium (e.g. EBM-2 medium (Clonetics, San Diego)) with 20% fetal calf serum and plated on fibronectin coated plates. Adhering cells are then expanded (preferably in the presence of a VEGF-B or a PDGF, 1-10 ug/ml of growth medium). Preferably, a sample from the cells is analyzed for cell surface molecules indicative of undifferentiated and differentiating progenitor cells.

Subjects are divided into two groups for mock surgery or surgery to implant or transplant a prosthesis or tissue or organ graft, such as a skin, bone, ligament, tendon, cartilage, vein or arterial graft. See Tepper, et al., “Endothelial Progenitor Cells: The Promise Of Vascular Stem Cells For Plastic Surgery,” Plastic and Reconstructive Surgery, 111:846-854 (2003). For the experimental group, the expanded progenitor cells are isolated from the plates and seeded into the surgical wounds, transplants or grafts (including synthetic grafts employing tissue engineering), or are reintroduced intravenously into the circulating blood. Control animals receive no cell therapy or cell therapy using nucleated cells isolated as described above, but without growth factor pretreatment and without growth factor-supplemented culture.

Animals are examined or sacrificed at various time points to evaluate the speed with which the wounds have healed and/or the success with which the body has accepted the graft or transplant.

### EXAMPLE 7

### VEGF-B, Chemotherapy, Bone Marrow Transplant Study

Four groups of test animals are established, one group will receive neither chemotherapy nor a bone marrow transplantation, one group will receive chemotherapy using one or more of the chemotherapeutic agents described below, one group will receive a bone marrow transplant, and one group will receive both chemotherapy and a bone marrow transplant. Subjects in each group will receive one or more of the following: VEGF-B₁₆₇, VEGF-B₁₈₆, a VEGF-B N-acetylated variant, PDGF-A, PDGF-B, PDGF-C, and PDGF-D or a control protein. Before administering the compounds, blood samples are collected, for white blood cell characterization as described in Example 1, and additionally with anti-PDGF-receptor-α and anti-PDGF-receptor-β antibodies.

Chemotherapeutic agents for use include the following: cyclophosphamide (5,725 mg/m2), cisplatin (165 mg/m2), and carmustine (BCNU) (600 mg/m2)—to be administered over a four-day period. Three hours after marrow reinfusion, the administration of the particular growth factor product or control protocol is initiated as a continuous intravenous (IV) infusion for 14 to 21 days, or as a second dose schedule administered as a daily four-hour infusion for up to 21 days. Growth factor product or control protein is administered at dosages of 0.1 to 100 μg/kg/day.

In addition to blood sampling every 2-3 days (with characterization of white blood cells as described in Example 1, and additionally with anti-PDGF-receptor-α and anti-PDGF-receptor-β antibodies used), bone marrow biopsies and marrow progenitor assays are performed at five-day intervals, and the functional characteristics of white blood cells are monitored before, during, and, if abnormal, after infusion.

### EXAMPLE 8

### PDGF-CC Mobilizes Endothelial Progenitors Upon Tissue Ischemia In Vivo

To examine the possible mechanism by which PDGF-CC (PDGF-CC refers to a homodimer of PDGF-C) stimulates vessel growth and maturation, the effects of PDGF-CC on vascular endothelial progenitor cells (EPCs) were assayed.

For the EPC mobilization assay, mice were treated with PDGF-CC protein (4.5 μg/day: an approximation based on 30 μg per week) using subcutaneously implanted osmotic minipumps (Alzet, type 2001) immediately after femoral (hind limb) artery ligation. After two or five days, mice were sacrificed and spleens harvested for EPC analysis using procedures described previously [Dimmeler, S., et al., J. Clin. Invest. 108:391-97 (2001); Asahara, T., et al., Circ. Res. 85:221-8 (1999)]. Spleens were mechanically minced using syringe plungers and laid over Ficoll to isolate splenocytes. Splenocytes were seeded into fibronectin-coated 24-well plates in 0.5 ml EBM medium. After three weeks of culturing, adherent cells were stained for Dil-Ac-LDL/lectin and number of the positive cells counted. Late outgrowth EPCs (after 3 weeks of culture) were identified by metabolic uptake of DiI-acetylated-LDL (Molecular Probes) and positive staining of Alexa 488-labeled isolectin B4 (Molecular Probes). Quantification of the EPC density was performed by confocal microscopy in five microscopic fields at 200× magnification, and average EPC density calculated.

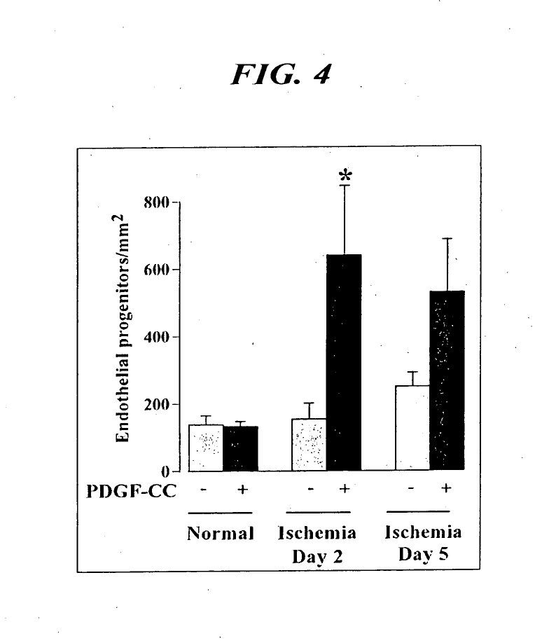

Specifically, EPC mobilization was quantified by counting the number of acLDL-DiI/isolectin-IB4 positive endothelial cells after 3 weeks of plating out spleen mononuclear cells. By scoring only after 3 weeks, only late-outgrowth EPCs, and not surviving sludged-off endothelial cells, are selectively assayed [Lin, Y., et al., J. Clin. Invest. 105:71-7. (2000); Rafii, S., J. Clin. Invest. 105:17-9 (2000)]. In baseline conditions, PDGF-CC did not affect mobilization of EPCs (EPCs/mm2: 139±26 in control versus 134+14 after PDGF-CC, n=6 each group, P=0.9, FIG. 4).

To look at the effect of PDGF-CC under ischemic conditions, femoral arteries were ligated. Treatment with PDGF-CC protein for two days (4.5 μg/day via minipump) in the mice augmented EPC mobilization approximately Four-fold above the levels found in the control group from day 2 to day 5 after hind limb ischemia (EPCs/mm2: 155±47 in control versus 641±207 after PDGF-CC, n=9,10, “*” P<0.05, FIG. 4; Values are presented as mean +\− SEM. of 10 mice.). This augmentation persisted to day five, albeit at a lower level (EPCs/mm²:249±42 in control versus 528±157 after PDGF-CC; n=10 each group, P=0.1, FIG. 4). Thus, PDGF-CC treatment enhanced EPC mobilization in tissue ischemia, thereby providing a source of ECs needed for revascularization of ischemic tissues.

PDGF-CC mediated EPC mobilization is an early and sustained event after tissue ischemia, starting at day 2 after ischemia and continuing onwards. This time window parallels the onset of ischemia-induced angiogenesis and thus leads to the possibility of an efficient launching of angiogenesis by providing sufficient amount of EPCs. The foregoing data demonstrate that PDGF-CC can be employed to mobilize EPCs at a time of active revascularization of ischemic tissues. In these experiments, PDGF-CC induced EPC mobilization was ischemia-dependent. EPC mobilization was increased by PDGF-CC in mice with hind limb ischemia, but not in normal ones, suggesting that PDGF-CC exerts its function in concert with other ischemia-dependent factors.

The hindlimb ligation and EPC migration assays described above were repeated using PDGF-AA, PDGF-BB, and PDGF-CC, as well as control vehicle. The model used was the Balb/c hind limb ischemia model as described in of Luttun, et al., Nat. Med. 8:831-40 (2002). The following modifications over the procedures described above were carried out: The concentration of the protein was 4.3 μg per day for each factor and the analysis was done after 2 days. The vehicle was PBS. An Alzet minipump 1003D, which works for two consecutive days was used instead of a minipump 2001, which works for seven consecutive days. The pump rate/drug delivery is exactly the same, and drug loading was calculated and performed in a way that it matched the 30 μg over 7 days strategy. EPC mobilization was assayed in ligated mice, at day 2 post-ligation; treatments continuously given via osmotic minipumps, blind analysis. EPC mobilization is expressed as density per mm²:vehicle (n=1): 119; PDGF-AA (n=7): 1052+/−177; PDGF-BB (n=8): 858+/−195; PDGF-CC (n=3): 1328+/−228. These results confirm those described above, and also demonstrate the superiority of PDGF-CC over PDGF-AA and PDGF-BB in mobilizing EPCs.

### EXAMPLE 9

### PDGF-CC Enhances Differentiation of Bone Marrow Progenitor Cells into Both Endothelial and Smooth Muscle Cells

Upon stimulation by growth factors or cytokines, bone marrow stem/progenitor cells can differentiate into ECs and SMCs and thereby contribute to angio/arteriogenesis [Orlic, D., et al., Nature 410:701-5 (2001); Kawamoto, A., et al., Circulation 103:634-7 (2001); Asahara, T., et al., Circ. Res. 85:221-8 (1999). The potential role of PDGF-CC in the differentiation of bone marrow stem/progenitor cells into vascular cells was investigated as follows.

A. Adherence Assay

To investigate this potential role of PDGF-CC, an adherence assay was first performed. Enriched human BM derived AC133+CD34+cells—a population enriched for stem/progenitor cells [Miraglia, S., et al., Blood 90:5013-21 (1997); Yu, Y., et al., AC133-2, J. Biol. Chem. 277:20711-6 (2002); Donnelly, D. S. & Krause, D. S., Leuk Lymphoma 40:221-34 (2001).]—(Clonetics) at 10⁵/ml were cultured for 3 days in HPGM (Clonetics) in a 6-well plate (Becton Dickinson). [Miraglia, S., et al., Blood 90:5013-21 (1997); Yu, Y., et al., AC133-2, J. Biol. Chem. 277:20711-6 (2002); Donnelly, D. S. & Krause, D. S., Leuk. Lymphoma 40:221-34 (2001)] Cells were then seeded in collagen coated 12-well plates in EBM (Clonetics) medium containing 4% FCS and VEGF₁₆₅ (R&D Systems) or PDGF-CC (50 ng/ml each). These cells expressed PDGFR-A, when analyzed by RT-PCR (not shown). Growth factors were added every two days and media were refreshed at 75% every four days.

For the adherence assay, 2.5×10⁴ of non-adherent cells/ml were cultured in the same conditions on chamber slides coated with collagen, or in a 96-well plate coated with 0.3% gelatin in PBS. Cells were then washed three times with PBS, fixed and stained with May-Grünwald Giemsa (Sigma) after two weeks of culture on chamber slides (Becton Dickinson). The number of viable cells were estimated by ATP quantification using cellTiter-glo luminescent cell viability assay (Promega) according to the manufacturer's instructions.

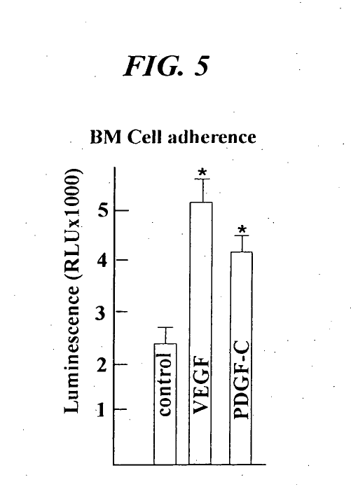

After two weeks of stimulation, both PDGF-CC and VEGF enhanced the cell adherence, a prerequisite for anchorage-dependent cell proliferation, differentiation, migration and prevention of apoptosis [Assoian, R. K. J Cell Biol 136, 1-4. (1997); Asahara, T. et al. Science 275, 964-7. (1997)] (FIG. 5; *: P<0.05. Values are presented as mean+/−SEM.)

B. Cell Differentiation Assays

The cell differentiation assays involved cell surface marker staining, cells (2×10⁴/well) cultured on collagen-coated culture slides for two, three, and four weeks were fixated (45 min, 25° C.) and permeabilized (45 min, 25° C.) using a Intrastain Kit (DAKO), and then labeled with CD31 FITC (Becton Dickinson), CD144 FITC (Pharmingen), CD34 FITC (Becton Dickinson) or SMC-Actin CY3 (Sigma). Single or double-labeled cells were analyzed using laser confocal immunofluorescence microscopy. The same kinds of cells and conditions as described in part “A” for the adherence assay were also used for the cell differentiation assays.

The results showed that PDGF-CC and VEGF markedly differed in their ability to induce the commitment of these stem cells into either the endothelial or smooth muscle cell lineages. After two weeks of stimulation, both PDGF-CC and VEGF induced the expression of EC surface markers CD 144 (VE-cadherin) and CD31 (PECAM)), indicating that these growth factors induced a characteristic endothelial phenotype. Vehicle-treated (control) cells remained negative for these markers. Only PDGF-CC additionally induced the expression of the smooth muscle cell marker SMA in a fraction of these cells, indicating that these cells had acquired a characteristic SMC phenotype. The VEGF-treated cells did not become SMA positive relative to background (nor did controls). Double labeling experiments revealed that PDGF-CC often induced the expression of CD31 and SMA in the same cells.

By four weeks, most (>95%) of the PDGF-CC-treated cells were SMA positive and had lost their expression of CD144 and CD31. In contrast, VEGF-treated cells were still CD144 and CD31 positive and remained SMA negative. Thus, PDGF-CC initially induced bone marrow progenitor cells to differentiate into cell types with both endothelial or smooth muscle cell characteristics—eventually, after long-term treatment, yielding cells with a SMC-like phenotype. PDGF-CC thus differed from VEGF, as the latter only caused bone marrow progenitors to acquire EC-specific markers, even after prolonged treatment.

Continuing the discussion of the significance of the finding that PDGF-CC mobilizes vascular stem/progenitor cells, increases stem cell adherence/viability, and promotes stem cell differentiation is the present discovery that PDGF-CC mediated BM cell differentiation is bi-directional, that is, both EPC- and SMC-oriented. The final destination of the stem cells probably depends on the cellular environment, and needs to be co-orchestrated by other growth factors or cytokines. In the presence of VEGF, which often is a sign of tissue ischemia, the BM cells may be better directed to their EC fate and contribute to the initial stage of angiogenesis-capillary formation. PDGF-CC may further strengthen the second stage of angiogenesis-vessel maturation, by providing SMCs to the capillaries and leading to a stabilized functional vasculature. Without high levels of VEGF, that is, in normoxia, PDGF-CC turns ultimately the BM cells into SMCs, thus avoiding the possibility of angioma-genesis (Angioma-genesis is discussed in Carmeliet, P. Nat Med 6, 1102-3. (2000).). Taken together, the early and ischemia-dependant EPC mobilization and the bi-directional BM cell differentiation conferred by PDGF-CC provide a valuable characteristic of both efficiency and safety for the growth factor's in vivo therapeutic usage in building new blood vessels to treat ischemic diseases.

Moreover, the angio/arteriogenic effect of PDGF-CC involves several mechanisms, including mobilization and differentiation of vascular progenitors, chemotactic effect on differentiated both ECs and SMCs, proliferation and migration of perivascular cells, and upregulation of VEGF expression. Thus, in contrast to VEGF or PDGF-AA and -BB, whose vascular effects are largely restricted to EC or SMC/fibroblast cells, respectively, the effect of PDGF-CC on the vasculature is more pleiotropic and thus allows for a more synchronized, universal action of the different cell types, needed to build functional blood vessels.

The abilities of PDGF-CC to mobilize vascular progenitors, by promoting their differentiation into both endothelial and smooth muscle cells, and stimulate these differentiated vascular cells, indicate that PDGF-CC is useful in vitro and in vivo orchestrating the complex process of building mature, durable and functional vessels.

### EXAMPLE 10

### PDGF-CC Promotes Endothelial Cell Migration and Microvessel Sprouting

In this example, the effect of PDGF-CC on EC migration and proliferation was compared to that of VEGF (which primarily affects endothelial cells [Senger, D. R., et al., Am. J. Pathol. 149:293-305. (1996)]) and PDGF-AA and -BB (which primarily affect fibroblasts and smooth muscle cells [Heldin, C. H. & Westermark, B. Physiol. Rev. 79:1283-1316 (1999)]). Migration, proliferation and aortic ring assays were performed.

A. Cell Migration Assays

Cell migration assays were performed on growth-arrested confluent HMVEC or BAEC cells. Cell monolayers were wounded with a rubber policeman and washed with serum-free medium. Dishes were then incubated for 20 hours in serum-free medium containing VEGF165, PDGF-AA, -BB (R&D Systems, Minneapolis USA) or PDGF-CC. Each assay included two dishes per condition and was repeated three times independently. Cells were photographed at 40× magnification, and migration percentage corresponding to the ratio between area of the cells and the total area of the wound (Biocom visiol@b 2000 version 4.52, San Diego). For the cell migration assay, ANOVA Dunett's test was used for data analyzing, with P<0.05 considered statistically significant. Data are presented as mean+\−SEM.

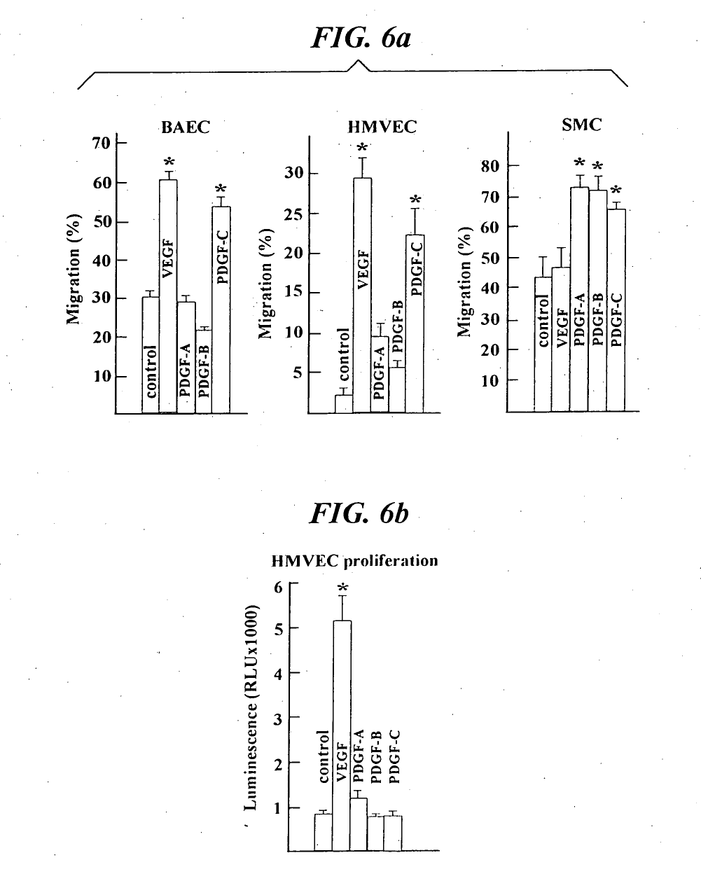

PDGFR-α expression on the human microvascular endothelial cells (HMVEC) was confirmed by Western blot, albeit at a lower level as compared with that of the SMCs (not shown). VEGF and PDGF-CC, but not PDGF-AA or PDGF-BB, stimulated migration of human microvascular endothelial cells (HMVEC) and bovine aorta endothelial cells (BAEC) (FIG. 6a).

B. Proliferation Assay

For HMVEC proliferation assay, cells were seeded in 96-well plates (5 wells per condition), and incubated with PDGF-AA, PDGF-BB or PDGF-CC (50 ng/ml) after serum starvation. After 7 days, viable cells were counted using cellTiter-glo luminescent cell viability assay (Promega). For NIH-3T3 and hSMC proliferation assay, cells cultured in 96-well plates were serum-starved overnight, followed by treatment with growth factors at different concentrations. Two days later, cell numbers were counted and proliferation percentage calculated, using cells cultured in medium containing 10% serum as control.

In contrast to the migration results, none of the PDGFs affected EC proliferation (FIG. 6b), in agreement with the previous observation that PDGFR-A does not transmit mitogen signals in ECs [Marx, et al., J. Clin. Invest. 93:131-9 (1994)], whereas VEGF dramatically induced EC proliferation (FIG. 6b; *: P<0.05. Values are presented as mean+/−SEM.).

C. Aortic Ring Assay

The aortic ring assay is a means of assessing outgrowth of microvessels from an intact vessel in vitro [Blacher, S., et al., Angiogenesis 4:133-42 (2001)]. The assay was performed as described in [Blacher, S., et al., Angiogenesis 4:133-42 (2001)]. Briefly, one-millimeter long aortic rings were embedded in gels of rat tail interstitial collagen and cultured at 37° C., supplemented with different growth factors (50 ng/ml). Experiments included three explants per condition and were repeated at least twice. Aortic rings were photographed at 25× magnification.

At day 9 after culturing, microvessels and the distance of their outgrowth from the aortic ring were quantified and evaluated using Student's t-test. Specifically, two-tailed Student's t-test was used for data analysis, with P<0.05 considered statistically significant. For cell migration assay, ANOVA Dunett's test was used for data analyzing, with P<0.05 considered statistically significant. Quantification of the outgrowth of microvascular sprouts and perivascular fibroblast-like cells was performed using computer-assisted morphometry.

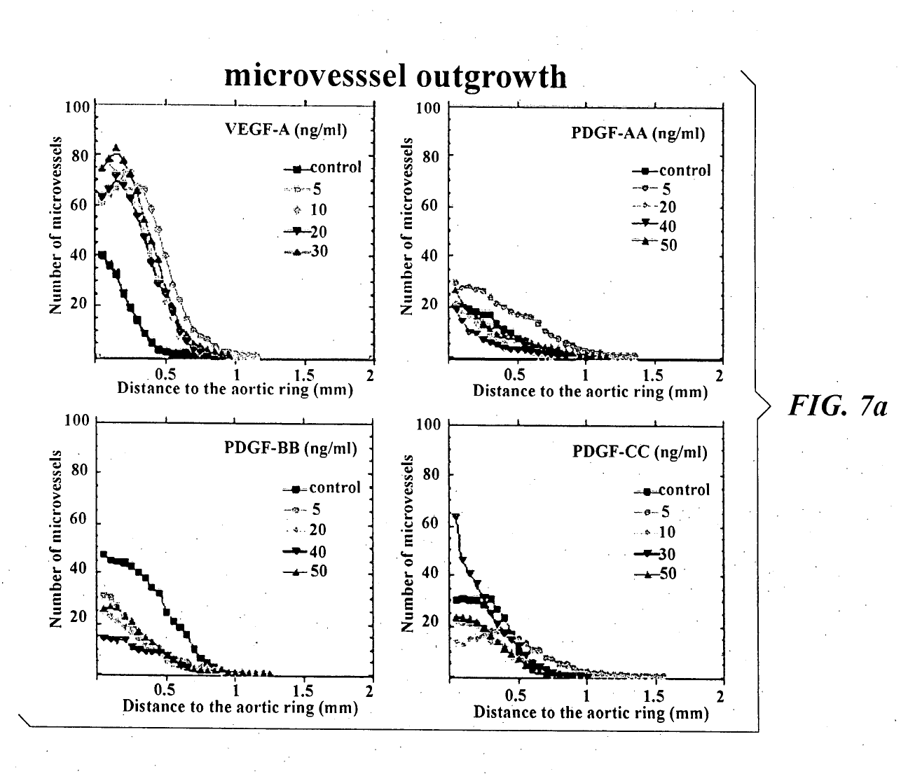

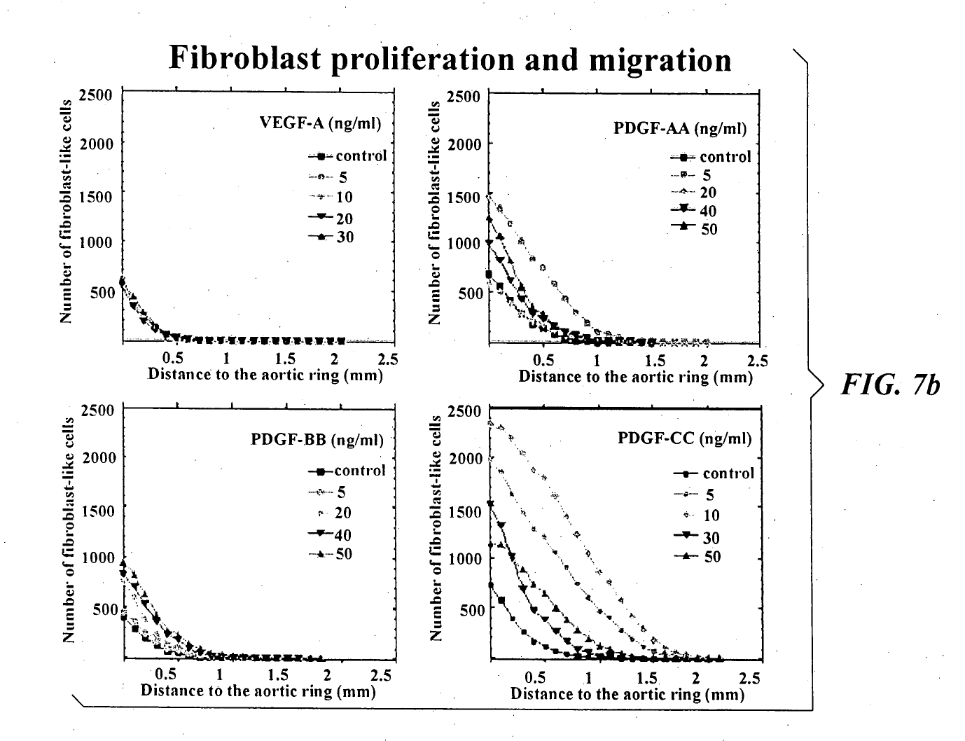

In baseline conditions, only a small number of microvessels sprouted from the aortic rings—most of them over very short distances (0.25 mm from the aortic ring) and only a small fraction (<5%) growing out over longer distances (>0.5 mm from the aortic ring). VEGF had the most potent effect on microvessel outgrowth. VEGF not only increased the number of sprouting microvessels (P<0.001 at all concentrations versus control), but also the distance over which they grew out (P<0.05 at all concentrations versus control; FIG. 7a, b).

PDGF-CC enhanced the outgrowth of both microvascular sprouts and fibroblast-like cells. At 5-10 ng/ml, PDGF-CC maximally stimulated perivascular fibroblast-like cells, which emigrated over much greater distances from the aortic ring. At high concentrations (30-50 ng/ml), PDGF-CC still stimulated fibroblast-like cell growth and emigration, but less significantly than at lower concentrations, possibly because the perivascular cells were recruited by the sprouting microvessels. PDGF-CC at 30 ng/ml increased the number of microvessels (P<0.001 versus control, FIGS. 7a, 7b) and increased the distance of vessel outgrowth at 5 ng/ml (P<0.01 versus control, FIGS. 7a, 7b). Unlike VEGF, which was ineffective on perivascular fibroblast-like cells, PDGF-CC increased the number and migration of the perivascular cells over much greater distances from the aortic ring, while PDGF-AA has an intermediate effect. Apparently, PDGF-CC had its maximum effect at 30 ng/ml on microvessel sprouting, and was less potent at a concentration of 50 ng/ml, indicating that the dose-response relationship of PDGF-CC in the aortic ring assay was bell-shaped. A similar bell-shaped dose-response relationship has been documented for other members of the VEGF/PDGF-superfamily [Jin, K. L., et al., J. Mol. Neurosci. 14:197-203 (2000)].

PDGF-AA and -BB had no effect on the number of microvessels (FIG. 7a), although they both increased the distance of vessel outgrowth at different concentrations (5 ng/ml for PDGF-AA and 20-50 ng/ml for PDGF-BB respectively, P<0.01 versus control, FIG. 7a). Thus, PDGF-CC mobilized EC migration in cultured cells and promoted microvessel sprouting in aortic ring assay. This chemotactic effect of PDGF-CC on ECs is surprising, because although the other PDGFs are among the most potent stimuli of mesenchymal cell migration, they either do not or only minimally stimulate EC migration. In certain conditions, they even inhibit EC migration. [Thommen, J Cell Biochem. 1997 Mar. 1;64(3):403-13; De Marchis, F., et al., Blood 99:2045-2053 (2002)]

### EXAMPLE 11

### PDGF-CC is Both Chemotactic and Mitogenic for Smooth Muscle Cells and Perivascular Fibroblast Cells

This example describes the mitogenic and chemotactic effects of PDGF-CC on SMCs and perivascular fibroblast cells, and compared the effect of PDGF-CC on such cells in different cellular environments—in both cultured cells and aortic ring assay, in comparison with VEGF, PDGF-AA and PDGF-BB.

A. Cell Migration Assay

Cell migration assays were performed as described in Example 10. In cell culture assay, all three PDGFs stimulated hSMCs migration with a comparable potency, while VEGF had no effect on SMC migration (FIG. 6a). Thus, interestingly, PDGF-CC promoted the migration of both ECs and SMCs, while VEGF only stimulated EC migration, and PDGF-AA, -BB only SMCs. This observation is consistent with the aortic ring assay, where PDGF-CC stimulated microvessel outgrowth while PDGF-AA and -BB were less effective.

B. Aortic Ring Assay

In the aortic ring assay (assay described in Example 10), the growth and emigration of perivascular fibroblasts from the intact vessel was quantified using computer-assisted image analysis after treatment with different PDGFs at different concentrations.

In baseline conditions, individual perivascular fibroblasts (identified as isolated cells, not associated with sprouting microvessels) were sparse and emigrated over only short distances from the aortic ring. PDGF-CC promoted the proliferation and migration of the fibroblast-like perivascular cells dramatically at all different concentrations tested, with an optimum concentration of 5-10 ng/ml. The mitogenic effect of PDGF-CC was much greater than those of PDGF-AA and -BB. VEGF had no mitogenic activity on the fibroblast-like cells. PDGF-CC significantly increased the number of fibroblasts, which also emigrated over much greater distances from the aortic ring (P<0.001 at all concentrations versus control, FIG. 7b). At high concentrations (30-50 ng/ml), PDGF-CC still stimulated fibroblast growth and emigration but less significantly than at lower concentrations, possibly because its effects were dose-dependent (see above) and/or the perivascular cells surrounded the sprouting microvessels. PDGF-AA had an intermediate effect (P<0.05 at different concentrations versus control, FIG. 7b). In contrast, VEGF had no and PDGF-BB only had a effect at a concentration of 50 ng/ml on perivascular fibroblast growth and emigration (P<0.05 in PDGF-BB versus control, FIG. 7b). Thus, of all PDGF homologues, PDGF-CC most significantly stimulated migration and proliferation of perivascular cells in the aortic ring assay—an assay that is believed to reflect more closely the in vivo situation and allows synergistic interactions between the different vascular cell types [Hartlapp, I. et al, Faseb J, 2001, 15: 2215-24; Blacher, S., et al. Angiogenesis 4:133-42 (2001); Nehls, V., et al., Cell Tissue Res. 270:469-74 (1992); Tille, J. C. & Pepper, M. S., Exp. Cell. Res. 280:179-91. (2002)]

C. Western Blot and Receptor Activation Assays

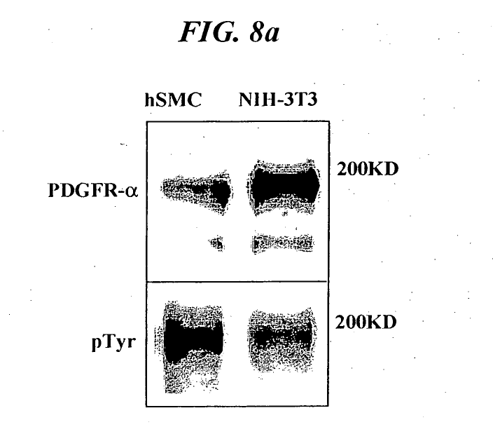

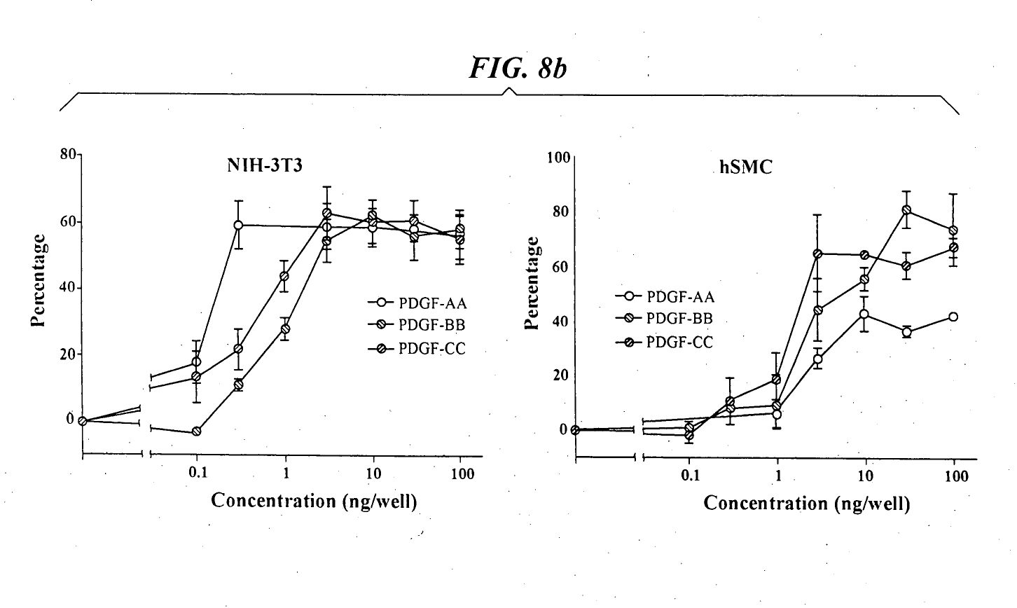

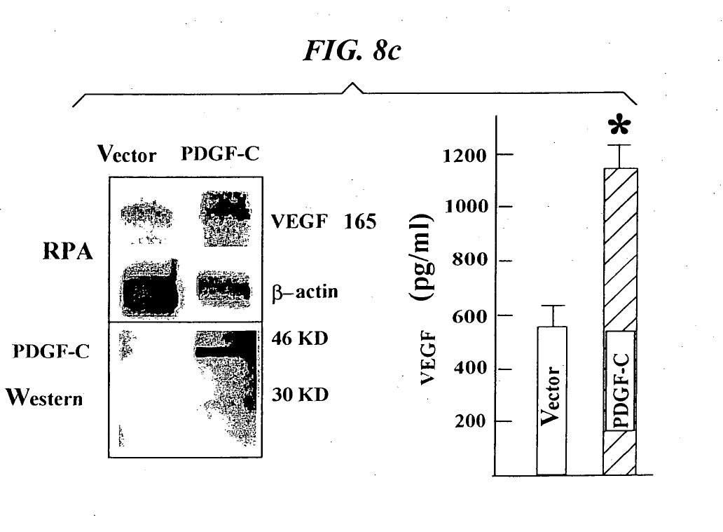

For Western blot assay, subconfluent cells were rinsed with cold PBS supplemented with 5 g/ml of antiprotease cocktail, lysed in RIPA buffer and analyzed on 10% acrylamide SDS PAGE in reducing condition. Two antibodies to PDGFR-α (rabbit polyclonal antibody, dilution: 1/500, Santa Cruz, sc431; and monoclonal peroxidase-labeled anti-rabbit antibody, dilution: 1/2500, Sigma, A-2074) were used for protein detection. Membranes were developed using the Supersignal System (Pierce). For receptor activation, and tissue/cell lysates were subjected to immunoprecipitation using the rabbit anti-PDGFR-α antibody. The precipitants were analyzed on SDS-PAGE, and immunoblotted using a monoclonal anti-phosphotyrosine antibody (PY99, Santa Cruz). PDGF-CC induced proliferation of hSMC and NIH3T3 cells, but not ECs. In cultured hSMC and NIH-3T3 fibroblast cells, in which PDGFR-α is highly expressed and activated (FIG. 8A; PDGFR-α was highly expressed (Western blot, upper lanes—anti-PDGFR-α) and activated/phosphorylated (lower lanes—anti-phosphotyrosine) in the hSMC and NIH-3T3 cells.). In cell culture system, PDGF-CC induced the proliferation of hSMC and NIH-3T3 fibroblast cells. All three PDGFs displayed about the same degree of mitogenic activity—with the effect of PDGF-CC on hSMC cells being slightly more pronounced. (FIG. 8b.)

### EXAMPLE 12

### PDGF-CC Upregulates VEGF Expression

Because the foregoing data indicates that PDGF-CC induced some VEGF-like effects, the ability of PDGF-CC to upregulate the expression of VEGF was examined. The initial results (of infarcted tissue using LAD ligation) suggested such an effect as they showed more prominent VEGF immunoreactivity in the border zones surrounding the infarcts after PDGF-CC treatment than control.

To further confirm the initial results, PDGF-C was overexpressed in NIH-3T3 fibroblast cells and VEGF expression was measured at both RNA and protein levels. For PDGF-C over-expression, mouse full-length PDGF-C cDNA was cloned into pcDNA3.1/zeo(+) mammalian expression vector (Invitrogen) and the construct was verified by sequencing. Plasmid DNA was transfected into semiconfluent cells using Lipofectamine plus reagent according to manufacturers protocol (Life technology). Stable transfectants were selected with 700 μg ml-1 Zeocin (Invitrogen) for 3 weeks. Resistant colonies were pooled and maintained in medium supplemented with 300 μg ml-1 Zeocin.

Over-expression of PDGF-C was confirmed by Western blotting (FIG. 8c, lower-left panel). For PDGF-CC Western blot assay, cells were starved in serum-free medium overnight. Conditioned media (overnight) were collected and protein concentration determined (Bradford, 1976). 35 μg of protein was trichloroacetic acid (TCA) precipitated and subjected to Western blot using affinity purified polyclonal rabbit antibodies against PDGF-CC [Li, et al., Nat. Cell. Biol. 2:302-09 (2000)]. All the samples were in triplicates and the experiment was repeated twice. Secreted VEGF protein was quantified using the Quantikine immunoassay kit (R&D system) according to the manufacturers protocol.

RNase protection analysis (RPA) was performed according to the manufacturer's protocol (Ambion) to investigate gene expressions at mRNA level. Riboprobes were prepared using RNA polymerase (Promega) and 32P-UTP (Amersham). Mouse β-actin cDNA (250 bp, Ambion) was used as an internal control. VEGF mRNA level was significantly upregulated in the PDGF-CC over-expressing (NIH-3T3) cells as compared to that of vector transduced cells by RPA assay (FIG. 8C, upper-left panels).

ELISA assay further confirmed that secreted VEGF protein level in the serum-free PDGF-CC over-expressing cell-conditioned media was significantly increased as compared with that of the vector-transduced cell conditioned (mock-transfected cell conditioned) media (VEGF in pg/ml: 1140±96 in PDGF-CC versus 585±80 in control, n=6, P<0.01, FIG. 8C, right panel. *: P<0.05. Values are presented as mean+/−SEM.). The activity of PDGF-CC to upregulate VEGF may explain, at least in part, some of its angiogenic activities.

### EXAMPLE 13

### PDGF-CC Stimulates Angiogenesis and Arteriogenesis in the Ischemic Heart

A previously established mouse model of myocardial ischemia was used to assess whether PDGF-CC is capable of stimulating the revascularization of ischemic myocardium. After coronary ligation, new vessels revascularize the ischemic core from its surrounding border region.

RNAse protection analysis revealed that PDGFR-α transcripts for the PDGF-C receptor (PDGFR-α) were detectable in the normal myocardium. β-actin was used as an internal control. Moreover, immunoprecipitation and subsequent Western blotting using an equal amount of protein extract revealed that PDGFR-α protein levels were significantly upregulated in the ischemic border zones surrounding the infarcts, i.e., where vessel growth is most active, as compared to the rest of the normal myocardium. PDGFR-α was activated more in the border zones than in the normal (non-ischemic) regions of the heart, and maximally after PDGF-CC treatment. PDGFR-α was, as assessed by Western blotting of the phosphorylated tyrosine residues after immunoprecipitation, highly activated in the border zone surrounding the infarcts.

Acute myocardial ischemia and hind limb ischemia mouse models that have been were previously described [Luttun, A. et all, Nat. Med. 8:831-40 (2002).; Heymans, S. et al. Nat Med 5, 1135-42 (1999).] were used in experiments. Subcutaneously implanted osmotic minipumps (Alzet, type 2001) were used for continuous protein delivery for 7 days. Human PDGF-CC core domain protein was produced as described. [Li, X. et al. Nat Cell Biol 2, 302-309 (2000).] Fluorescent or color dye microspheres (yellow, 15 μm, Molecular Probes) were administered after maximal vasodilatation (sodium nitroprusside, 50 ng/ml, Sigma) for blood flow measurement, and flow was calculated as described. [Carmeliet, P. et al. Nat Med 5, 495-502. (1999).] For histology, the hearts were harvested seven days after LAD (left anterior descending coronary artery) ligation, and sectioned longitudinally (6 μm). Infarcted areas were morphologically inspected after immunohistochemistry staining using thrombomodulin (rabbit anti-TM, for all vessels) and smooth muscle alpha-actin (mouse anti-SMA, for mature SMC covered vessels, Dako), and vessel densities calculated. Gastrocnemius muscles after femoral artery ligation were sectioned transversally and analyzed after H&E or immunostainings with the EC marker CD31 (PECAM, rat anti-CD31, Pharmingen). Vessel densities and tissue necrosis/regeneration in the gastrocnemius muscle were analyzed morphmometrically using the KS300 image analysis soft ware (Zeiss). Remodeling of collateral vessels in the upper hind limb after femoral ligation was quantified as reported. [Luttun, A. et al. Nat. Med. 8:831-40 (2002).] Rabbit polyclonal VEGF antibody (Santa Cruz, sc-152) was used for immunohistochemistry staining.

To examine whether PDGF-CC could stimulate revascularization of the ischemic myocardium recombinant human PDGF-CC core domain protein was delivered using a minipump, continuously over one week after coronary ligation. PDGF-CC protein treatment increased vascular density in the infarcted areas in a dosage dependent way. Compared to control, PDGF-CC also increased the amount of active PDGFR-α in the border region. After seven days, angiogenesis was quantified by counting the number of endothelial cell (EC)-lined vessels in the ischemic area after immunolabeling with thrombomodulin (TM). Vessel maturation (arteriogenesis) was evaluated by counting the arterioles, immunoreactive for smooth muscle cell β-actin (SMA). At 1.5 μg/day, PDGF-CC minimally affected the TM-positive vessel density (vessels/mm2: 175±8 in control, n=21 versus 190+13 after 10 μg PDGF-CC, n=7; P═NS), but increased, by 1.36-fold, the number of SMA-positive arterioles (vessels/mm2: 40±9 in control, n=21 versus 54±4 after 10 μg PDGF-CC; n=7; P<0.05). When using a 3-fold higher dose (4.5 μg/day), PDGF-CC significantly stimulated angiogenesis (TM-positive vessels/mm2: 175±8 in control versus 230±22 after PDGF-CC; n=10-21; P<0.05) and arteriogenesis (SMA-positive vessels/mm2: 40±9 in control versus 58+7 after PDGF-CC; n=10-21; P<0.05).

No signs of hemorrhage, edema or fibrosis were observed in the PDGF-CC treated hearts. These new vessels were functional as perfusion of the ischemic myocardial region was significantly increased by 1.4-1.7 fold (blood flow in ml/min/g: infarct: 1.6±0.2 in control versus 2.2±0.2 after 30 μg PDGF-CC; n=7-9; P<0.05; normal part of the infarcted heart: 2.0±0.2 in control versus 3.3±0.5 after 30 μg PDGF-CC; n=7-9; P<0.05. Blood flow such that 30 μg is approx. to be 4.5 μg/day: 1.6+0.2 ml/min/g in control versus 2.2±0.2 ml/min/g after PDGF-CC; n=7-9; P<0.05). The effect of PDGF-CC to stimulate revascularization appeared to be restricted to the ischemic heart, as no differences were observed in vessel density in other organs (average blood flow of left and right kidney in ml/min/g: 5.2±0.4 in control versus 5.7±0.5 after 30 μg PDGF-CC; n=7-9; P=0.5).

The magnitude of revascularization of the ischemic myocardium induced by PDGF-CC is comparable to that of VEGF and PlGF. The mice tolerated the PDGF-CC treatment without problems, appeared healthy and had no signs of toxicity (weight loss, inactivity). Thus, PDGF-CC protein treatment promoted functional revascularization in cardiac ischemia via enhanced angiogenesis (more vessels) and arteriogenesis (more SMC coverage). The angio/arteriogenic activity of PDGF-CC in cardiac ischemia is surprising, because the other PDGFR-α ligand, PDGF-AA is poorly angiogenic or even suppresses angiogenesis.

### EXAMPLE 14

### Therapeutic Angiogenesis with PDGF-CC in Ischemic Limbs PDGF-CC Stimulates Angiogenesis in the Ischemic Limb

To further verify the angio/arteriogenic activity of PDGF-CC in vivo, the effect of PDGF-CC in an established mouse model of hind limb ischemia was also investigated. For the model see Luttun, A. et al. Nat. Med. 8:831-40 (2002). For more on limb ischemia, a common disease in humans see J Control Release 78, 285-94. (2002); Beckman, J. A., Creager, M. A. & Libby, P. Jama 287, 2570-81. (2002). PDGFR-α expression was first quantified by RNAse protection analysis, using β-actin as an internal control (ratio of PDGFR-A levels were normalized to the β-actin control), in the gastrocnemius muscle, which becomes highly ischemic after ligation of the femoral artery. [Deindl, E. et al. Circ Res 89, 779-86. (2001); Couffinhal T et al. American Journal of Pathology 152, 1667-1679 (1998).] Two days after femoral artery ligation, when a fraction of myocytes died due to ischemic necrosis, PDGFR-α transcript levels decreased to 76% of those found in normal muscles (PDGFR-α/β-actin transcript levels: 1.27+/−0.06 in normal muscle versus 0.96+/−0.05 after ligation, n=9, 10; P<0.01). However, compared to vehicle, a daily treatment with 4.5 μg PDGF-CC upregulated PDGFR-A expression at day 2 after ligation and almost completely restored its expression levels to those found in the unligated control muscle (PDGFR-α/β-actin transcript levels: 1.16+0.08 after PDGF-CC versus 0.96±0.05 in untreated, n=10 each group; P<0.05).

Revascularization of the ischemic gastrocnemius muscle, which only occurred in those regions where regenerating muscle replaced the necrotic avascular muscle, was scored after continuous delivery, by osmotic minipump, of 4.5 μg PDGF-CC per day for one week after femoral artery ligation. PDGF-CC protein treatment increased the PECAM+ capillary and SMA+ arteriolar density in the ischemic gastrocnemius muscles. PDGF-CC protein treatment decreased muscle necrosis and increased muscle regeneration in the gastrocnemius muscle at seven days after femoral artery ligation. Necrotic muscle fibers were identified as ghost cells lacking nuclei and containing a hyaline cytosol; regenerating myocytes were identified as small cells with central nuclei. Areas are expressed as percentage of the total muscle area. Treatment with PDGF-CC after femoral artery ligation not only increased angiogenesis (e.g. the capillary density; PDGFR-α/β-actin transcript levels: 1.16±0.08 after PDGF-CC versus 0.96±0.05 in untreated, n=10 each group; P<0.05), it also enhanced arteriogenesis (e.g. the density of SMA+vessels; SMA positive vessels/mm2: 53.1+/−3.7 after PDGF-C vs 38.6+/−4.8 after saline, n=15,16, P=0.02.). Moreover, PDGF-CC enhanced skeletal muscle regeneration (regenerating/total muscle area: 14±3% in control versus 27±4% after PDGF-CC, n=15, 16, P<0.05) and, as a result, also reduced the extent of ischemic muscle necrosis (necrotic/total muscle area: 80±3% in control versus 65+5% after PDGF-CC, n=15, 16, P<0.05;), suggesting that muscle regeneration and angiogenesis might be linked. PDGF-CC also enlarged the second-generation collateral side branches in the adductor muscle (680±40 μm³ after saline versus 920±100 μm³ after PDGF-CC; N=10; P=0.05). No signs of hemorrhage, edema or fibrosis were observed in the PDGF-CC treated limbs. Muscle regeneration was maximal at sites of intense angiogenesis, suggesting that both processes were linked. PDGF-CC minimally affected the remodeling of the collateral vessels in the adductor muscle, presumably because of potential ischemia-dependant effect of PDGF-CC and this region is not ischemic after femoral artery ligation [Deindl, et al. Circ Res 89, 779-86 (2001); Pu, et al., J Invest Surg., 7(1): 49-60 (1994)]. Thus, PDGF-CC stimulates revascularization in mouse models of both heart and limb ischemia.

PDGF-CC was found to increase the perfusion of the ischemic myocardium by revascularizing the myocardium not only with SMC-covered coronary vessels (providing bulk flow) but also with endothelial-lined capillaries (distributing the flow to the individual cardiomyocytes). In the ischemic limb, PDGF-CC was also found to stimulate both angiogenesis and arteriogenesis. Moreover, the observation that PDGF-CC also enhanced muscle regeneration in areas of active revascularization further underscores that the new vessels were functional and perfused. The pleiotropic activity of PDGF-CC may also explain why no side effects of hemangioma-genesis and edema formation after PDGF-CC treatment were observed, which has been observed after VEGF administration.

PDGF-CC treatment mobilized endothelial progenitors and increased the vessel density and blood perfusion in the ischemic heart and limb, but did not affect quiescent vessels in other organs. Although PDGF-CC enlarged the second-generation side branches of the collateral vessels in the adductor muscle, this growth factor has, overall, a less dramatic effect on the remodeling of the preexisting collaterals in the upper limb region after femoral artery ligation than, for instance, bFGF, PlGF or GM-CSF. However, the molecular and cellular mechanisms of the growth of collateral vessels are quite distinct from those determining the formation of new capillaries and their maturation by coverage with smooth muscle cells. In particular, not ischemia but shear stress-induced recruitment of monocytes/macrophages is well known to play a critical role in initiating collateral growth in the upper hindlimb and PDGF-CC does not affect their recruitment (data not shown). Because only the lower, but not the upper limb is ischemic after femoral artery ligation, PDGF-C seems to be involved more in ischemia-dependent angiogenesis than in the shear stress-induced collateral remodeling.

Muscle regeneration was improved after femoral artery ligation by PDGF-CC, especially in regions where vascular regeneration was also maximal.

The foregoing description and examples have been set forth merely to illustrate the invention and are not intended to be limiting. Because modifications of the disclosed embodiments incorporating the spirit and substance of the invention may occur to persons skilled in the art, the invention should be construed to include everything within the scope of the appended claims and equivalents thereof.

1-94. (canceled)

95. A method of stimulating stem cell recruitment, proliferation, or differentiation to stimulate myelopoiesis comprising,
identifying a mammalian subject in need of stem cell recruitment, proliferation, or differentiation to treat, prevent, or reduce myelopsuppression, and
administering to the mammalian subject a composition comprising a vascular endothelial growth factor B (VEGF-B) product, in an amount effective to stimulate myelopoiesis in the subject.

96. The method of claim 95, wherein the mammalian subject is human.

97. The method of claim 95, wherein the identifying comprises selecting a subject selected from the group consisting of:
(a) a subject undergoing antineoplastic chemotherapy;
(b) a bone marrow transplant subject; and
(c) a subject undergoing antineoplastic radiation therapy.

98. The method of claim 97, wherein the administering comprises administering the composition contemporaneously with, or after, administering at least one of the antineoplastic chemotherapy, the bone marrow transplant, and the antineoplastic radiation therapy.

99. The method of claim 95, wherein the identifying comprises:
measuring circulating white blood cells or bone-marrow derived stem cells in the subject to screen for myelosuppression.

100. The method of claim 99, wherein the measuring comprises measuring at least one of CD34+ stem cells and hematopoietic stem cells.

101. The method of any one of claims 95, wherein the method further comprises monitoring the number of circulating white blood cells or bone-marrow derived stem cells after administration of the composition.

102. The method of claim 101, wherein the monitoring comprises detection of at least one cell surface marker selected from the group consisting of VEGFR-1, VEGFR-2, and CD34.

103. The method of any one of claims 95, further comprising administering to said subject an agent selected from the group consisting of:
(a) granulocyte colony stimulating factor (G-CSF), macrophage-CSF (M-CSF), granulocyte-macrophage-CSF (GM-CSF), interleukin-3 (IL-3), stem cell factor (SCF), vascular endothelial growth factor (VEGF), vascular endothelial growth factor C (VEGF-C), vascular endothelial growth factor D (VEGF-D), platelet derived growth factor A (PDGF-A), platelet derived growth factor B (PDGF-B), platelet derived growth factor C (PDGF-C), platelet derived growth factor D (PDGF-D), and placental growth factor (PlGF);
(b) a polynucleotide comprising a nucleotide sequence encoding any member of (a), and
(c) combinations thereof.

104. The method of claim 95, wherein the VEGF-B product comprises a VEGF-B polypeptide.

105. The method of claim 104, wherein the VEGF-B is glycosylated.

106. The method of claim 95, wherein the VEGF-B product comprises a polynucleotide that encodes a VEGF-B polypeptide.

107. The method of claim 106, wherein the VEGF-B product comprises a viral vector containing the polynucleotide.

108. The method of claim 107, wherein the vector comprises a replication-deficient adenoviral or adeno-associated viral vector.

109. The method of claim 104, wherein the VEGF-B polypeptide comprises the amino acid sequence of SEQ ID NO: 2, SEQ ID NO: 4, or a fragment thereof that binds VEGFR-1.

110. The method of claim 104, wherein the VEGF-B polypeptide is associated as a heterodimer with a VEGF polypeptide.

111. The method of claims 104, wherein the VEGF-B polypeptide binds VEGFR-1 and is encoded by a polynucleotide that hybridizes under stringent conditions with the complement of the polynucleotide in SEQ ID NO: 1 or 3.

112. The method of claim 95, wherein the VEGF-B product further comprises a pharmaceutically acceptable carrier.

113. A method of stimulating stem cell proliferation or differentiation, comprising,
obtaining a biological sample from a mammalian subject, wherein said sample comprises stem cells, and
contacting the stem cells with a composition comprising a vascular endothelial growth factor B (VEGF-B) product.

114. The method according to claim 113, further comprising a step of purifying and isolating the stem cells after obtaining the sample and before the contacting step.

115. The method according to claim 113, further comprising a step of purifying and isolating the stem cells after the contacting step.

116. The method according to claim 115, wherein the purified stem cells comprise stem cells selected from the group consisting of VEGFR-1+stem cells, CD34+stem cells, CD133+stem cells, and combinations of the same.

117. The method according to claims 113, wherein the contacting comprises culturing the stem cells in a culture containing the VEGF-B product.

118. The method according to any one of claims 113, further comprising a step of returning the stem cells to the mammalian subject.

119. The method according to any one of claims 113, further comprising a step of transplanting the cells into a different mammalian subject.

120. The method of claim 118, wherein the cells are seeded into a tissue, organ, or artificial matrices ex vivo, and said tissue, organ, or artificial matrix is attached, implanted, or transplanted into the mammalian subject.

121. The method of claim 119, wherein the cells are seeded into a tissue, organ, or artificial matrices ex vivo, and said tissue, organ, or artificial matrix is attached, implanted, or transplanted into the mammalian subject.

122. The method according to claim 113, wherein the mammalian subject is human.

123. The method according to claim 122, wherein the human subject needs antineoplastic chemotherapy, and wherein the biological sample is obtained prior to administering a dose of chemotherapy, and wherein the stem cells are returned to the human subject after the contacting and after the dose of chemotherapy.

124. The method according to any one of claims 113, wherein the VEGF-B product comprises a VEGF-B polypeptide.

125. The method of claim 124, wherein the VEGF-B is glycosylated.

126. The method of claim 113, wherein the VEGF-B product comprises a polynucleotide that encodes a VEGF-B polypeptide.

127. The method of claim 126, wherein the VEGF-B product comprises a viral vector containing the polynucleotide.

128. The method of claim 127, wherein the vector comprises a replication-deficient adenoviral or adeno-associated viral vector.

129. The method of claim 124, wherein the VEGF-B polypeptide comprises the amino acid sequence of SEQ ID NO: 2, SEQ ID NO: 4, or a fragment thereof that binds VEGFR-1.

130. The method of claim 124, wherein the VEGF-B polypeptide is associated as a heterodimer with a VEGF polypeptide.

131. The method of claims 124, wherein the VEGF-B polypeptide binds VEGFR-1 and is encoded by a polynucleotide that hybridizes under stringent conditions with the complement of the polynucleotide in SEQ ID NO: 1 or 3.

132. The method of claim 113, wherein the VEGF-B product further comprises a pharmaceutically acceptable carrier.

133. A method of stimulating stem cell recruitment, proliferation, or differentiation comprising,
identifying a mammalian subject in need of stem cell recruitment, proliferation, or differentiation to treat or prevent ischemia, and
administering to the subject a composition comprising a platelet derived growth factor (PDGF) product.

134. The method of claim 133, wherein the subject is human.

135. The method of claim 133, wherein the PDGF product comprises at least one member selected from the group consisting of PDGF-A, PDGF-B, PDGF-C, and PDGF-D products.

136. The method of claim 133, wherein the PDGF product binds PDGFR-α.

137. The method of claim 133, wherein the PDGF product comprises at least a PDGF-C product.

138. The method of claim 133, wherein the PDGF product comprises a PDGF polypeptide.

139. The method of claim 133, wherein the PDGF product comprises a polynucleotide that encodes a PDGF polypeptide.

140. The method of claim 139, wherein the PDGF product comprises a viral vector containing the polynucleotide.

141. The method of claim 140, wherein the vector comprises a replication-deficient adenoviral or adeno-associated viral vector.

142. The method of claim 138, wherein the PDGF polypeptide comprises a portion of the amino acid sequence set forth in SEQ ID NO: 7 or 9 that is effective to bind PDGFR-alpha or PDGFR-beta.

143. The method of claim 138, wherein the PDGF polypeptide binds PDGFR-alpha or PDGFR-beta and is encoded by a polynucleotide that hybridizes under stringent conditions with the complement of the polynucleotide in SEQ ID NO: 6 or 8.

144. The method claim 133, wherein the PDGF polypeptide comprises a member selected from the group consisting of a PDGF-A polypeptide, a PDGF-B polypeptide, a PDGF-C polypeptide, a PDGF-D polypeptide, combinations thereof, or fragments thereof that bind to at least one of PDGF receptors alpha and beta (PDGFR-alpha, PDGFR-beta).

145. The method of claims 133, wherein the PDGF polypeptide comprises a PDGF-C or PDGF-D polypeptide or a fragment thereof that binds to at least one of PDGF receptors alpha and beta (PDGFR-alpha, PDGFR-beta).

146. The method of claims 133, wherein the composition further comprises a pharmaceutically acceptable carrier.

147. The method of claims 133, further comprising administering to said subject an agent selected from the group consisting of:
(a) granulocyte colony stimulating factor (G-CSF), macrophage-CSF (M-CSF), granulocyte-macrophage-CSF (GM-CSF), interleukin-3 (IL-3), stem cell factor (SCF), vascular endothelial growth factor (VEGF), vascular endothelial growth factor B (VEGF-B) vascular endothelial growth factor C (VEGF-C), vascular endothelial growth factor D (VEGF-D), platelet derived growth factor A (PDGF-A), platelet derived growth factor B (PDGF-B), platelet derived growth factor C (PDGF-C), platelet derived growth factor D (PDGF-D), and placental growth factor (PlGF);
(b) a polynucleotide comprising a nucleotide sequence encoding any member of (a), and
(c) combinations thereof.

148. A method of stimulating stem cell proliferation or differentiation, comprising,
obtaining a biological sample from a mammalian subject, wherein said sample comprises stem cells, and
contacting the stem cells with a composition comprising a platelet derived growth factor C (PDGF-C) product or platelet derived growth factor D (PDGF-D) product.

149. The method according to claim 148, further comprising contacting the cells with at least one additional PDGF product selected from the group consisting of a PDGF-A product, a PDGF-B product, a PDGF-C product and a PDGF-D product.

150. The method according to claim 148, further comprising a step of isolating the stem cells after obtaining the sample and before the contacting step.

151. The method according to claim 148, further comprising a step of purifying and isolating the stem cells after the contacting step.

152. The method according to claim 151, wherein the purified stem cells comprise cells that express PDGFR-alpha.

153. The method according to claim 151, wherein the purified stem cells comprise CD34+stem cells.

154. The method according to claim 148, wherein the contacting comprises culturing the stem cells in a culture containing the PDGF-C product or PDGF-D product.

155. The method according to claim 148, further comprising a step of returning the stem cells to the mammalian subject after the contacting step.

156. The method according to claim 148, further comprising a step of transplanting the cells into a different mammalian subject after the contacting step.

157. The method of claim 155, wherein the cells are seeded into a tissue, organ, or artificial matrix ex vivo, and said tissue, organ, or artificial matrix is attached, implanted, or transplanted into the mammalian subject.

158. The method of claim 156, wherein the cells are seeded into a tissue, organ, or artificial matrix ex vivo, and said tissue, organ, or artificial matrix is attached, implanted, or transplanted into the mammalian subject.

159. The method according to claims 148, wherein the mammalian subject is human.

160. The method according to claim 159, wherein the human subject needs antineoplastic chemotherapy, and wherein the biological sample is obtained prior to administering a dose of chemotherapy, and wherein the stem cells are returned to the human subject after the contacting and after the dose of chemotherapy.

161. The method of claim 148, wherein the PDGF-C product or PDGF-D product comprises a PDGF-C polypeptide or PDGF-D polypeptide.

162. The method of claim 148, wherein the product comprises a polynucleotide that encodes a PDGF-C polypeptide or a PDGF-D polypeptide.

163. The method of claim 162, wherein the product comprises a viral vector containing the polynucleotide.

164. The method of claim 163, wherein the vector comprises a replication-deficient adenoviral or adeno-associated viral vector.

165. The method of claims 161, wherein the polypeptide comprises a portion of the amino acid sequence set forth in SEQ ID NO: 7 or 9 that is effective to bind PDGFR-alpha or PDGFR-beta.

166. The method of claim 161, wherein the PDGF polypeptide binds PDGFR-alpha or PDGFR-beta and is encoded by a polynucleotide that hybridizes under stringent conditions with the complement of the polynucleotide in SEQ ID NO: 6 or 8.

167. The method of claim 148, wherein the composition further comprises a pharmaceutically acceptable carrier.

168. The method of claim 148, further comprising administering to said subject an agent selected from the group consisting of:
(a) granulocyte colony stimulating factor (G-CSF), macrophage-CSF (M-CSF), granulocyte-macrophage-CSF (GM-CSF), interleukin-3 (IL-3), stem cell factor (SCF), vascular endothelial growth factor (VEGF), vascular endothelial growth factor B (VEGF-B), vascular endothelial growth factor C (VEGF-C), vascular endothelial growth factor D (VEGF-D), platelet derived growth factor A (PDGF-A), platelet derived growth factor B (PDGF-B), platelet derived growth factor C (PDGF-C), platelet derived growth factor D (PDGF-D), and placental growth factor (PlGF);
(b) a polynucleotide comprising a nucleotide sequence encoding any member of (a), and
(c) combinations thereof.

169. The method of claim 150, wherein the isolating comprises isolating AC133+/CD34+ cells from the biological sample.

170. The method according to claim 148, wherein the contacting comprises contacting the stem cells with the composition until stem cells differentiate into CD144+ cells.

171. The method according to claim 148, wherein the contacting comprises contacting the stem cells with the composition until stem cells differentiate into SMA+/CD144−/CD31−/CD34− cells.

172. The method according to claim 148, wherein the composition further comprises a VEGF-A product.

173. The method according to claim 148, wherein the contacting comprises culturing the stem cells in a culture containing the PDGF-C product.

174. The method according to claim 148, further comprising a step of returning the stem cells to the mammalian subject after the contacting step.

175. The method of claim 174, wherein the cells are seeded into a tissue, organ, or artificial matrix ex vivo, and said tissue, organ, or artificial matrix is attached or implanted into the mammalian subject.

176. The method according to claim 148, further comprising a step of transplanting the cells into a different mammalian subject after the contacting step.

177. The method of claim 176, wherein the cells are seeded into a tissue, organ, or artificial matrix ex vivo, and said tissue, organ, or artificial matrix is attached or transplanted into the different mammalian subject.

178. The method according to claim 148, wherein the mammalian subject is human.

179. The method according to claim 178, wherein the human subject has an ischemic condition.

180. The method of claim 161, wherein the polypeptide comprises an amino acid sequence at least 95% identical to SEQ ID NO: 7 or 9 and binds to at least one receptor selected from PDGFR-alpha and PDGFR-beta.

181. The method of claim 161, wherein the polypeptide comprises an amino acid sequence at least 95% identical to the amino acid sequence of SEQ ID NO: 10 and binds to and/or activates at least one receptor selected from PDGFR-α/α and PDGFR-α/β.

182. The method of claim 180, wherein the PDGF-C polypeptide binds PDGFR-α and is encoded by a polynucleotide that hybridizes under stringent conditions with the complement of the polynucleotide in SEQ ID NO: 6.

183. The method of claim 161, wherein the polypeptide binds PDGFR-alpha or PDGFR-beta and is encoded by a polynucleotide that hybridizes under stringent conditions with the complement of the polynucleotide of SEQ ID NO: 6 or 8.

184. A method of stimulating stem cell proliferation or differentiation, comprising,
obtaining a biological sample from a mammalian subject, wherein said sample comprises stem cells;
contacting a first aliquot of the stem cells with a first composition comprising a first growth factor product selected from a VEGF-B product and PDGF-C product; and
contacting a second aliquot of the stem cells with a second composition comprising a second growth factor product independently selected from the group consisting of VEGF-A, VEGF-B, VEGF-C, VEGF-D, PDGF-A, PDGF-B, PDGF-C, and PlGF products,
wherein the first and second growth factor products are not the same.

185. The method of claim 184, wherein the first growth factor product is a PDGF-C product and the second growth factor product is a VEGF-A product.

186. A method of promoting differentiation of stem cells into both endothelial and smooth muscle cells, comprising:
obtaining a biological sample from a mammalian subject, wherein said sample comprises stem cells; and
contacting the cells with a composition comprising a platelet-derived growth factor-C (PDGF-C) product, in an amount and for a time sufficient to cause the cells to differentiate into both endothelial and smooth muscle cells.

187. The method according to claim 184, further comprising returning the cells to the mammalian subject after the contacting.

188. The method of claim 187, wherein the mammalian subject has an ischemic condition.

189. A method of ameliorating an ischemic condition comprising:
(a) diagnosing a mammalian subject with an ischemic condition;
(b) isolating a biological sample from the mammalian subject, wherein the biological sample comprises stem cells;
(c) contacting the cells with a composition comprising a platelet-derived growth factor-C (PDGF-C) product, in an amount and for a time sufficient to cause the cells to differentiate into both endothelial and smooth muscle cells; and
(d) returning the cells to the mammalian subject.

190. The method according to claim 189, wherein the returning comprises implanting or injecting the cells into or adjacent to ischemic tissue of the mammalian subject.
